SEQUENCE LISTING (235 sequences)

SEQ ID NO: 1 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 2 - protein, 10 aa, synthetic construct
ARMRYNYFDY

SEQ ID NO: 3 - protein, 10 aa, synthetic construct
ARVKWDLLDY

SEQ ID NO: 4 - protein, 12 aa, synthetic construct
AREQWEPLYFDY

SEQ ID NO: 5 - protein, 12 aa, synthetic construct
AHLAHWGPYFDY

SEQ ID NO: 6 - protein, 14 aa, synthetic construct
ARFSNWGSGGYFDY

SEQ ID NO: 7 - protein, 12 aa, synthetic construct
ARERWALLFFEY

SEQ ID NO: 8 - protein, 12 aa, synthetic construct
AREKWELLYFDY

SEQ ID NO: 9 - protein, 12 aa, synthetic construct
ARERWELLFFDY

SEQ ID NO: 10 - protein, 12 aa, synthetic construct
AREKWVQLYFDF

SEQ ID NO: 11 - protein, 14 aa, synthetic construct
ARFSNWGSGGYFDY

SEQ ID NO: 12 - protein, 10 aa, synthetic construct
ARMRYNYFDY

SEQ ID NO: 13 - protein, 11 aa, synthetic construct
ARVVRDYGMDV

SEQ ID NO: 14 - protein, 12 aa, synthetic construct
ARERWELLFFDY

SEQ ID NO: 15 - protein, 12 aa, synthetic construct
AREKWELLYFDY

SEQ ID NO: 16 - protein, 12 aa, synthetic construct
AREKWVQLYFDF

SEQ ID NO: 17 - protein, 12 aa, synthetic construct
AREQWELLYFDY

SEQ ID NO: 18 - protein, 16 aa, synthetic construct
ARGGVGRNYYYYGMDV

SEQ ID NO: 19 - protein, 13 aa, synthetic construct
ARDGRWELLAFDI

SEQ ID NO: 20 - protein, 10 aa, synthetic construct
AKMRYNYFDY

SEQ ID NO: 21 - protein, 10 aa, synthetic construct
AKMRYNYFDY

SEQ ID NO: 22 - protein, 12 aa, synthetic construct
AREQWELLYFDY

SEQ ID NO: 23 - protein, 12 aa, synthetic construct
AREQWELLYFDY

SEQ ID NO: 24 - protein, 10 aa, synthetic construct
ARMRYNYFDY

SEQ ID NO: 25 - protein, 10 aa, synthetic construct
ARERYNYFDY

SEQ ID NO: 26 - protein, 10 aa, synthetic construct
ARERYNYFDY

SEQ ID NO: 27 - protein, 10 aa, synthetic construct
ARERYNYFDY

SEQ ID NO: 28 - protein, 10 aa, synthetic construct
ARERYNYFDF

SEQ ID NO: 29 - protein, 10 aa, synthetic construct
AREPYNYFDY

SEQ ID NO: 30 - protein, 10 aa, synthetic construct
ARERYNYFDY

SEQ ID NO: 31 - protein, 10 aa, synthetic construct
ARERYNYFDY

SEQ ID NO: 32 - protein, 10 aa, synthetic construct
ARERYNYFDY

SEQ ID NO: 33 - protein, 10 aa, synthetic construct
ARERYNYFDS

SEQ ID NO: 34 - protein, 11 aa, synthetic construct
ARVRRNYGMDV

SEQ ID NO: 35 - protein, 11 aa, synthetic construct
ARVRRNYGMDV

SEQ ID NO: 36 - protein, 11 aa, synthetic construct
ARVRRNYGMDV

SEQ ID NO: 37 - protein, 12 aa, synthetic construct
ARERWELLYFDY

SEQ ID NO: 38 - protein, 11 aa, synthetic construct
ARVRRNYGMDV

SEQ ID NO: 39 - protein, 11 aa, synthetic construct
ARVRRNYGMDV

SEQ ID NO: 40 - protein, 11 aa, synthetic construct
ARVRRNYGMDV

SEQ ID NO: 41 - protein, 11 aa, synthetic construct
ARVRRNYDMDV

SEQ ID NO: 42 - protein, 11 aa, synthetic construct
ARVRRNYGMDV

SEQ ID NO: 43 - protein, 12 aa, synthetic construct
AREQWVLLYFDY

SEQ ID NO: 44 - protein, 11 aa, synthetic construct
ARVRRNYGMDV

SEQ ID NO: 45 - protein, 11 aa, synthetic construct
ARVRRNYGMDV

SEQ ID NO: 46 - protein, 11 aa, synthetic construct
ARVRRNYGMDV

SEQ ID NO: 47 - protein, 12 aa, synthetic construct
ARERWELLYFDY

SEQ ID NO: 48 - protein, 12 aa, synthetic construct
ARERKYPLQFDY

SEQ ID NO: 49 - protein, 12 aa, synthetic construct
VRERKYPLYFDY

SEQ ID NO: 50 - protein, 11 aa, synthetic construct
ARVRRNYGMDV

SEQ ID NO: 51 - protein, 11 aa, synthetic construct
ARVRRNYGMDV

SEQ ID NO: 52 - protein, 12 aa, synthetic construct
ARERKYPLQFDY

SEQ ID NO: 53 - protein, 12 aa, synthetic construct
ARERKYLLYFDY

SEQ ID NO: 54 - protein, 11 aa, synthetic construct
ARVRRNYGMDV

SEQ ID NO: 55 - protein, 11 aa, synthetic construct
ARVRRNYGMDV

SEQ ID NO: 56 - protein, 11 aa, synthetic construct
ARVRRNYDMDV

SEQ ID NO: 57 - protein, 12 aa, synthetic construct
ARERKYLLYFDY

SEQ ID NO: 58 - protein, 12 aa, synthetic construct
SRERWNLLFFDY

SEQ ID NO: 59 - protein, 12 aa, synthetic construct
ARELWELLYFDY

SEQ ID NO: 60 - protein, 9 aa, synthetic construct
SYDSSNVVF

SEQ ID NO: 61 - protein, 10 aa, synthetic construct
QAWDSSTHVV

SEQ ID NO: 62 - protein, 9 aa, synthetic construct
SYDSSNRVF

SEQ ID NO: 63 - protein, 10 aa, synthetic construct
CSYAGSSTLI

SEQ ID NO: 64 - protein, 11 aa, synthetic construct
QVWDSSSDHPV

SEQ ID NO: 65 - protein, 9 aa, synthetic construct
SFDRSNRVF

SEQ ID NO: 66 - protein, 9 aa, synthetic construct
SYDSSNRVF

SEQ ID NO: 67 - protein, 9 aa, synthetic construct
SYDSSNRVF

SEQ ID NO: 68 - protein, 9 aa, synthetic construct
SYDSSNRVF

SEQ ID NO: 69 - protein, 11 aa, synthetic construct
QVWDSTSDHPV

SEQ ID NO: 70 - protein, 9 aa, synthetic construct
SYDSSNVVF

SEQ ID NO: 71 - protein, 10 aa, synthetic construct
SYDSSNHWVF

SEQ ID NO: 72 - protein, 9 aa, synthetic construct
SYDSSNRVF

SEQ ID NO: 73 - protein, 9 aa, synthetic construct
SYDSSNRVF

SEQ ID NO: 74 - protein, 9 aa, synthetic construct
SYDSSNRVF

SEQ ID NO: 75 - protein, 9 aa, synthetic construct
SYDSSNRVF

SEQ ID NO: 76 - protein, 11 aa, synthetic construct
GTWDSSLSAVV

SEQ ID NO: 77 - protein, 11 aa, synthetic construct
QSADTSGTYRV

SEQ ID NO: 78 - protein, 9 aa, synthetic construct
SYDGSNVVF

SEQ ID NO: 79 - protein, 9 aa, synthetic construct
SYDGSNVVF

SEQ ID NO: 80 - protein, 9 aa, synthetic construct
SYDSSNRVF

SEQ ID NO: 81 - protein, 9 aa, synthetic construct
SYDRSNRVF

SEQ ID NO: 82 - protein, 9 aa, synthetic construct
SYDSSNVVF

SEQ ID NO: 83 - protein, 9 aa, synthetic construct
SYDSSNRVF

SEQ ID NO: 84 - protein, 9 aa, synthetic construct
SYNGNNRVF

SEQ ID NO: 85 - protein, 9 aa, synthetic construct
SYDNSNRVF

SEQ ID NO: 86 - protein, 9 aa, synthetic construct
SYDGSNRVF

SEQ ID NO: 87 - protein, 9 aa, synthetic construct
SYDNSNRVF

SEQ ID NO: 88 - protein, 9 aa, synthetic construct
SYDSSNRVF

SEQ ID NO: 89 - protein, 9 aa, synthetic construct
SYDNNNRVF

SEQ ID NO: 90 - protein, 9 aa, synthetic construct
SYDNSNRVF

SEQ ID NO: 91 - protein, 9 aa, synthetic construct
SYDGNNRVF

SEQ ID NO: 92 - protein, 10 aa, synthetic construct
SYDSSNHWVF

SEQ ID NO: 93 - protein, 10 aa, synthetic construct
SYDSSNHWVF

SEQ ID NO: 94 - protein, 9 aa, synthetic construct
SYDSSNWVF

SEQ ID NO: 95 - protein, 9 aa, synthetic construct
SYDSSNRVF

SEQ ID NO: 96 - protein, 10 aa, synthetic construct
SYDSNNHWVF

SEQ ID NO: 97 - protein, 10 aa, synthetic construct
SYDSSNHWVF

SEQ ID NO: 98 - protein, 10 aa, synthetic construct
SYDSSDHWVF

SEQ ID NO: 99 - protein, 9 aa, synthetic construct
SYDSGNWVF

SEQ ID NO: 100 - protein, 9 aa, synthetic construct
SYDSNNWVF

SEQ ID NO: 101 - protein, 9 aa, synthetic construct
SYDSSNRVF

SEQ ID NO: 102 - protein, 10 aa, synthetic construct
YDSSNHWVFG

SEQ ID NO: 103 - protein, 10 aa, synthetic construct
SYDSSNHWVF

SEQ ID NO: 104 - protein, 10 aa, synthetic construct
SYDSSNHWVF

SEQ ID NO: 105 - protein, 9 aa, synthetic construct
SYDSSNRVF

SEQ ID NO: 106 - protein, 9 aa, synthetic construct
SYDSSNRVF

SEQ ID NO: 107 - protein, 9 aa, synthetic construct
SYDSSVRVF

SEQ ID NO: 108 - protein, 9 aa, synthetic construct
SYDSSNWVF

SEQ ID NO: 109 - protein, 10 aa, synthetic construct
SYDSSNHWVF

SEQ ID NO: 110 - protein, 9 aa, synthetic construct
SFDSSNRVF

SEQ ID NO: 111 - protein, 9 aa, synthetic construct
SYDRSNRVF

SEQ ID NO: 112 - protein, 10 aa, synthetic construct
SYDSSNHWVF

SEQ ID NO: 113 - protein, 10 aa, synthetic construct
SYDSSNHWVF

SEQ ID NO: 114 - protein, 10 aa, synthetic construct
SYDSSNHWVF

SEQ ID NO: 115 - protein, 9 aa, synthetic construct
SFDNSNRVF

SEQ ID NO: 116 - protein, 9 aa, synthetic construct
SYDSSNRVF

SEQ ID NO: 117 - protein, 9 aa, synthetic construct
SYDSNNRVF

SEQ ID NO: 118 - protein, 117 aa, synthetic construct
EVQLVESGGGLVKPGGSLRLSCAASGFTFSRYSMNWVRQTPGKGLEWVSSISNSCNYIYYADSVKGRFTISRDSAKNSLYLQMNSLRAEDTAVYYCARMRYNYFDYWGQGTLVTVSS

SEQ ID NO: 119 - protein, 117 aa, synthetic construct
EVQLVESGGGLVQPGGSLRLSCVASGFTFSNYWMNWVRQAPGKGLEWVANIKKDGSEKYYVDSVKGRFTISRDNAKNSLYLQMNSLRAEDTAVYYCARVKWDLLDYWGQGTLVTVSS

SEQ ID NO: 120 - protein, 119 aa, synthetic construct
EVQLVESGGGLVKPGGSLRLSCAASGFNLSTYSMNWVRQAPGKGLEWVSSISRSSSYIYYADAVKGRFTISRDNAQNSLYLQMNSLRAEDTAVYYCAREQWEPLYFDYWGQGTLVTVSS

SEQ ID NO: 121 - protein, 120 aa, synthetic construct
QITLKESGPTLVKPTQTLTLTCSFSGFSFTTSGVGVGWIRQPPGKALECLALIYWNDDKRYSPSLKNRLTITKDTSKNQVVLTMTNMDPVDTATYFCAHLAHWGPYFDYWGQGTLVTVSS

SEQ ID NO: 122 - protein, 121 aa, synthetic construct
EVLLVESGGGLVKPGGSLRLSCEASGFTFSRYSMNWVRQAPGKGLEWVSSISRSSRYIYYADSVKGRFTISRDNAKNSLYLQMISLRAEDTAVFYCARFSNWGSGGYFDYWGQGTLVTVSS

SEQ ID NO: 123 - protein, 119 aa, synthetic construct
EVQLVESGGGLVKPGGSLRLSCAASGFTVNNYSMNWVRQAPGKGLEWVSSISRSSNYIYYADSVKGRFTISRDNAKNSLYLQMNSLRAEDTAVYYCARERWALLFFEYWGQGTLVTVSS

SEQ ID NO: 124 - protein, 119 aa, synthetic construct
EVQLVESGGGLVKPGGSLRLSCAASGFNLSTYSMNWVRQAPGKGLEWVSSISRSSSYIYYADSVKGRFTISRDNAQNSLYLQMNSLRAEDTAVYYCAREKWELLYFDYWGQGTLVTVSS

SEQ ID NO: 125 - protein, 119 aa, synthetic construct
EVQLVESGGGLVKPGGSLRLSCAASGFTFSSYSMNWVRQAPGKGLEWVASMSRSSSYIYYAHSVKGRFTISRDNAKNSLYLQLNSLRAEDTAVYYCARERWELLFFDYWGQGTLVTVSS

SEQ ID NO: 126 - protein, 119 aa, synthetic construct
EVQLVESGGGLVKPGGSLRLSCAASGFTLSSYNMDWVRQAPGKGLEWVSSISRSSNYIYYADSVKGRFTISRDNAENSLYLQMNSLRAEDTAVYYCAREKWVQLYFDFWGQGTLVTVSS

SEQ ID NO: 127 - protein, 121 aa, synthetic construct
EVQLVESGGGLVKPGGSLRLSCAASGFTFRSYSMNWVRQAPGKGLEWVSSISRSSSYIYYADSVKGRFTISRDNAKNSLYLQMNSLRAEDTAVYYCARFSNWGSGGYFDYWGQGTLVTVSS

SEQ ID NO: 128 - protein, 117 aa, synthetic construct
EVQLVESGGGLVKPGGSLRLSCAASGFTFSRYSMNWVRQTPGKGLEWVSSISSSSNYIYYADSVKGRFTISRDNAKNSLYLQMNSLRAEDTAVYYCARMRYNYFDYWGQGTLVTVSS

SEQ ID NO: 129 - protein, 118 aa, synthetic construct
EVQLVESGGGLGQPGGSLRLSCAASGFTFSSYSMNWVRQAPGKGLEWVSYISRSSSTIYYADSVKGRFIISRDKAKNSLYLQMNSLRDEDTAVYYCARVVRDYGMDVWGQGTTVTVSS

SEQ ID NO: 130 - protein, 119 aa, synthetic construct
EVQLVESGGGLVKPGGSLRLSCAASGFTLSSYSMNWVRQAPGKGLEWVSTMSRSSSYIYYADSVKGRFTISRDNAKNSLYLQMNSLRAEDTAVYYCARERWELLFFDYWGQGTLVTVSS

SEQ ID NO: 131 - protein, 119 aa, synthetic construct
EVQLVESGGGLVKPGGSLRLSCAASGFTISRYSMNWVRQAPGKGLEWVSSISRSSSHIYYADSVKGRFTISRDNAKNSMYLQMNSLRAEDTAVYYCAREKWELLYFDYWGQGTLVTVSS

SEQ ID NO: 132 - protein, 119 aa, synthetic construct
EVQLVESGGGLVKPGGSLRLSCAASGFTLSSYSMDWVRQAPGKGLEWVSSISRSSNYIYYADSVKGRFTISRDNAENSLYLQMNSLRAEDTAVYYCAREKWVQLYFDFWGQGTLVTVSS

SEQ ID NO: 133 - protein, 119 aa, synthetic construct
EVQLVEFGGGLVRPGGSLRLSCVASGFTFSIFTMNWVRQAPGKGLEWVSTSSRSTSYIYYADSVKGRFTISRDNAKNSLYLQMNSLRAEDTAVYYCAREQWELLYFDYWGQGTLVTVSS

SEQ ID NO: 134 - protein, 123 aa, synthetic construct
QVQLVQSGAEVKKPGSSVKVSCKASGGTFSSYAISWVRQAPGQGLEWMGGIIPIFATANYAQKFQGRVTITADKSTSTAYMELSSLRSEDTAVYYCARGGVGRNYYYYGMDVWGQGTTVTVSS

SEQ ID NO: 135 - protein, 120 aa, synthetic construct
EVQLVESGGGLVQPGGSLRLSCAASGFTFSSYGMNWVRQAPGKGLEWVSYISRSSRTRYYADSVKGRFTISRDNAKNSLYLQMNSLRDEDTSVYYCARDGRWELLAFDIWGQGTMVTVSS

SEQ ID NO: 136 - protein, 117 aa, synthetic construct
EVQLVESGGGLVKPGGSLRLSCAASGFTFSRYSINWVRQAPGKGLEWVSSISSSSSYIYYADSLKGRFTISRDNARNSLYLQMNSLRAEDTAVYYCAKMRYNYFDYWGQGALVTVSS

SEQ ID NO: 137 - protein, 117 aa, synthetic construct; identical to SEQ ID NO: 136

SEQ ID NO: 138 - protein, 119 aa, synthetic construct; identical to SEQ ID NO: 133

SEQ ID NO: 139 - protein, 119 aa, synthetic construct
EVQLVESGGGLVKPGGSLRLSCAASGFTFSRYSINWVRQAPGKGLEWVSSSSRSSSYIYYADSVKGRFTISRDNAKNSLYLQMNSLRAEDTAVYYCAREQWELLYFDYWGQGTLVTVSS

SEQ ID NO: 140 - protein, 117 aa, synthetic construct
EVQLVESGGGLVKPGGSLRLSCGASGFTFSSYSMNWVRQAPGKGLEWVSSISSSSRYIYYADSLRGRFTISRDNAKNSLYLQMNSLRAEDTAVYYCARMRYNYFDYWGQGTLVTVSS

SEQ ID NO: 141 - protein, 117 aa, synthetic construct
EVQLVESGGGLVKPGGSLRLSCAASGFTFSRYSMNWVRQAPGKGLEWVSSISGSSRYIYYADSVKGRFTVSRDNAKNSLYLQMSSLSPEDTAVYYCARERYNYFDYWGQGTLVTVSS

SEQ ID NO: 142 - protein, 117 aa, synthetic construct
EVQLVESGGGLVKPGGSLRLSCAASGFTFSIYSMNWVRQAPGKGLEWVSSISKTSTFIFYADSVKGRFTISRDNAKNSLYLQMNSLSAEDTAVYYCARERYNYFDYWGQGTLVTVSS

SEQ ID NO: 143 - protein, 117 aa, synthetic construct
EVQLVESGGGLVKPGGSLRLSCAASGFTFSRYSLNWVRQAPGKGLEWVSSISSSSRYIFYADSVQGRFTISRDNAKNSLYLQMNSLRAEDTAVYFCARERYNYFDYWGQGTLVTVSS

SEQ ID NO: 144 - protein, 117 aa, synthetic construct
EVQLVESGGGLVKPGGSLRLSCAASGFTFSRYSMNWVRQAPGKGLEWVSSISSSSRYIFYADSVKGRFTISRDNAKNSLYLQMNSLRAEDTAVYYCARERYNYFDFWGQGTLVTVSS

SEQ ID NO: 145 - protein, 117 aa, synthetic construct
EVQLVESGGGLVKPGGSLRLSCAASGFTFSRYSMNWVRQAPGKGLEWVSSISSSSRYIFYADSVKGRFTMSRDNAKNSLYLQMNNLRAEDTAVYYCAREPYNYFDYWGQGTLVSVSS

SEQ ID NO: 146 - protein, 117 aa, synthetic construct
EVHLVESGGGLVKPGGSLRLSCAASGFTFSRYSMNWVRQAPGKGLEWVSSISSSSRYIYYADSVKGRFTISRDNAKNSLYLQMNSLRAEDTAVYYCARERYNYFDYWGQGTLVTVSS

SEQ ID NO: 147 - protein, 117 aa, synthetic construct
EVQLVESGGGLVKPGGSLRLSCAASGFTFSRYSLNWVRQAPGKGLEWVSSISSSSRYIYYADSVKGRFTISRDNAKNSLYLQMNSLRAEDTAVYYCARERYNYFDYWGQGTLVTVSS

SEQ ID NO: 148 - protein, 117 aa, synthetic construct
EVQLVESGGGLVKRGGSLRLSCATSGFTLSRYSMNWVRQAPGKGLEWVSSISSSSSYIYYADSVKGRFTISRDNAKNSLYLQMNSLRAEDTALYYCARERYNYFDYWGQGTLVTVSS

SEQ ID NO: 149 - protein, 117 aa, synthetic construct
EVQLVESGGGLVKPGGSLRLSCAASGFTFSIYSMNWVRQAPGKGLEWVSSISKSSRYIFYADSVKGRFTISRDNAKNSLYLQMNSLSAEDTAVYYCARERYNYFDSWGQGTLVTVSS

SEQ ID NO: 150 - protein, 118 aa, synthetic construct
EVQLVESGGGSVQPGGSLRLTCAASGFTFSTYSMNWVRQAPGKGLEWVSYISRSSSTIYYADSVKGRFTISRDNAKNSLYLQMNSLRDEDTAVYYCARVRRNYGMDVWGQGTTVTVSS

SEQ ID NO: 151 - protein, 118 aa, synthetic construct
EVQLVESGGGLVQPGGSLRLSCAASGFTFSSYSMNWVRQAPGKGLEWVSYISRSTSTIYYADSVKGRFTISRDNAKNSLYLQMNSLRDEDTAVYYCARVRRNYGMDVWGQGTTITVSS

SEQ ID NO: 152 - protein, 118 aa, synthetic construct
EVQLVESGGGLVQPGGSLRLSCAASGFTFSSYSMNWVRQAPGKGLEWVSYISRSSSTIYYADSVKGRFTISRDNAKNSLYLQMNSLRDEDTAVYYCARVRRNYGMDVWGQGTTVTVSS

SEQ ID NO: 153 - protein, 119 aa, synthetic construct
EVQLVESGGGLVKPGGSLRLSCAASGFTFSSYSMNWVRQAPGKGLEWVSSSSRSSSYIYYADSVKGRFTITRDNAKNSLYLQMNSLRAEDTAVYYCARERWELLYFDYWGQGTLVTVSS

SEQ ID NO: 154 - protein, 118 aa, synthetic construct
EVQLVESGGGLVQPGGSLRLSCAASGFTFSSYSMNWVRQAPGKGLEWVSYISRSSNTIYYADSVKGRFTISRDNARNSLYLQMSSLRDEDAAVYYCARVRRNYGMDVWGQGTTVTVSS

SEQ ID NO: 155 - protein, 118 aa, synthetic construct
EVQLVESGGGLVQPGGSLRLSCAASGFTFSSYSMNWVRQAPGKGLEWVSYISSSGSTIYYADSVKGRFTISRDNAKSSLYLQMNSLRDEDTAVYYCARVRRNYGMDVWGQGTTVTVSS

SEQ ID NO: 156 - protein, 118 aa, synthetic construct
EVQLVESGGGLVQPGGSLRLSCVVSGFTFSSYSMNWVRQAPGKGLEWVSYISSSHSTIYYADSVKGRLTISRDNAKNSLYLQMNSLRDEDTAVYYCARVRRNYGMDVWGQGTTVTVSS

SEQ ID NO: 157 - protein, 118 aa, synthetic construct
EVQLVESGGGLVQPGGSLRLSCAASGFTFSSYSMNWVRQAPGKGLEWVSYISSSINTIYYADSVKGRFTISRDNAKNSLYLQMNSLRDEDTAVYYCARVRRNYDMDVWGQGTTVTVSS

SEQ ID NO: 158 - protein, 118 aa, synthetic construct
EVQLVESGGGLVQPGGSLRLSCAASGFTFSSYSVNWVRQAPGKGLEWVSYISRSSSIIYYVDSVKGRFTISRDNAKNSLYLQMNSLRDEDTAVYYCARVRRNYGMDVWGQGTTVTVSS

SEQ ID NO: 159 - protein, 119 aa, synthetic construct
EVQLVESGGGLVKPGGSLRLSCAASGFILNNYSMNWVRQAPGKGLEWVSSISRSSSYIYYADSVKGRFTISRDNAKNSLYLQMNSLRADDTAVYYCAREQWVLLYFDYWGQGTLVTVSS

SEQ ID NO: 160 - protein, 118 aa, synthetic construct
EAQLVESGGGLVQPGGSLRLSCAASGFTFSSYSMNWVRQAPGKGLEWVSYISRSSSTIYYADSAKGRFTISRDNAKNSLYLQMNSLRDEDTAVYYCARVRRNYGMDVWGQGTTVTVSS

SEQ ID NO: 161 - protein, 118 aa, synthetic construct
EVQLVESGGGLVQPGESLRLSCAASGFTFSSYSMNWVRQAPGKGLEWVSYISRSSNTIYYADSVKGRFTISRDNARNSLYLQMNSLRDEDTAVYYCARVRRNYGMDVWGQGTTVTVSS

SEQ ID NO: 162 - protein, 118 aa, synthetic construct
EVQLVESGGGLVQPGGSLRLSCAVSGFTFSSYSMNWVRQAPGKGLEWVSYSSSSSSTIYYADSVKGRFTISRDNAKNSVYLQMNSLRDEDTAVYYCARVRRNYGMDVWGQGTTVTVSS

SEQ ID NO: 163 - protein, 119 aa, synthetic construct
EVHLVESGGGLVKPGGSLRLSCAASGITFSRYSMNWVRQAPGKGLEWVSSISRSSNYIYYADSVKGRFTISRDNAKNSLYLQMNSLRAEDTAVYYCARERWELLYFDYWGQGTLVTVSS

SEQ ID NO: 164 - protein, 120 aa, synthetic construct
QVQLQESGPGLVKPSQTLSLTCTVSGGSITSGASYWSWIRQHPGKGLEWIGYIYYSGSTYHSPSLKSRVTISVDTSKNRFSLMLSSVTAADTAVYYCARERKYPLQFDYWGQGTLVTVSS

SEQ ID NO: 165 - protein, 120 aa, synthetic construct
QVQLQESGPGLMKPSQTLSLTCTVSGGSISSGVYYWNWIRQHPGKGLEWIGDIYYSGSTYYNPSLKSRVTISVDTSKNQFSLKLSSVTAADTAVYYCVRERKYPLYFDYWGQGTLVTVSS

SEQ ID NO: 166 - protein, 118 aa, synthetic construct
EVQLVESGGGLVQPGGSLRLSCAASGFTFSSYSMNWVRQAPGKGLEWVSYISSSSSTIYYADSVKGRFTISRDNAKNSLFLQMNSLRDEDTAVYYCARVRRNYGMDVWGQGTTVTVSS

SEQ ID NO: 167 - protein, 118 aa, synthetic construct
EVQLVESGGGLVQPGGSLRLSCAASGLTFSRYSMNWVRQAPGKGLYWVSYISTSSTTIYYADSVKGRFTISRDNAKNSLSLQMNSLRDEDTAVYYCARVRRNYGMDVWGQGTTVTVSS

SEQ ID NO: 168 - protein, 120 aa, synthetic construct
QVQLQESGPGLVEPSQTLSLICTVSGGSISSGASYWSWIRQHPGKGLEWIGYIYYSGTTYFNPSLKSRVTISLDTSKNQFSLKLSSVTAADTAVYYCARERKYPLQFDYWGQGTLVTVSS

SEQ ID NO: 169 - protein, 120 aa, synthetic construct
QVQLQESGPGLVKPSQTLSLTCTVSGGSISSGAYCWSWIRQHPGKGLEWIGSIYYSGSTYYNPSLKSRVTISVDTSKKQFSLKLSSVTAADTAVYYCARERKYLLYFDYWGQGTLVTVSS

SEQ ID NO: 170 - protein, 118 aa, synthetic construct
EMQLVESGGGLVQRGGSLRLSCAASKFTFSGYAMNWVRQAPGKGLEWVSYISSSTSTTYYADSVKGRFTISRDNAKNSLYLQMNSLRDEDTAVYYCARVRRNYGMDVWGQGTTVTVSS

SEQ ID NO: 171 - protein, 118 aa, synthetic construct
EVQLVESGGGLVQPGGSLRLSCAASGFTFSRYSINWVRQAPGKGLEWVSYISSNTSTIYYADSVKGRFTISRDNAKNSLYLQMNSLRDEDTAVYYCARVRRNYGMDVWGQGTTVTVSS

SEQ ID NO: 172 - protein, 118 aa, synthetic construct
EVQLVGSGGGLVQPGGSLRLSCAASGFTFSSYSMNWVRQAPGKGLEWVSYISSSSSTIYYADSVKGRFTISRDNAKNSLYLQMNSLRDEDTAVYYCARVRRNYDMDVWGQGTTVTVSS

SEQ ID NO: 173 - protein, 120 aa, synthetic construct
QVQLRESGPGLVKPSQTLSLTCTVSGGSINSGGYCWSWIRQHPGKGLEWIGSIYYSGSTYYNPSLKSRVTISVDTSKNQFSLKLSSVTAADTAVYYCARERKYLLYFDYWGQGTLVTVSS

SEQ ID NO: 174 - protein, 119 aa, synthetic construct
EVQLVESGGGLVKPGGSLRLSCAASGFTFSSYCMNWVRQAPGKGLEWVSSISRSSSYIYYADSVKGRFTISRDNAKNSLYLQMNSLRAEDTAVYYCSRERWNLLFFDYWGQGTLVTVSS

SEQ ID NO: 175 - protein, 119 aa, synthetic construct
EVQLVESGGGLVKPGGSLRLSCAASGFTFSSYSINWVRQAPGKGLEWVSSISRSSSYIYYADSVKGRFTISRDNAKNSLYLQMNSLRAEDTAVYYCARELWELLYFDYWGQGTLVTVSS

SEQ ID NO: 176 - protein, 110 aa, synthetic construct
NFMLTQPHSVSESPGKTVTISCTRSSGSIASNYVQWYQQRPGSSPTTVVYEDNQRPSGVPDRFSGSIASSSNSASLTISGLKTEDEADYYCQSYDSSNVVFGGGTKLTVL

SEQ ID NO: 177 - protein, 107 aa, synthetic construct
SYELTQPPSVSVSPGQTASITCSGDKLGHKYVCWYQQKPGQSPVLVIYQDNKRPSGIPERFSGSNSGNTATLTISGTQAMDEADYYCQAWDSSTHVVFGGGTKLTVL

SEQ ID NO: 178 - protein, 110 aa, synthetic construct
NVMLIQPHSVSESPGKTVTISCTRSSGSIASKYVQWYQQRPGSSPTTVIYEDNQRPSGVPDRFSGSIDSSSNSASLTISGLKTEDEADYYCQSYDSSNRVFGGGTKLTVL

SEQ ID NO: 179 - protein, 110 aa, synthetic construct
QSALTQSASVSGSPGQSITISCTGTSSDVGSYNLVSWYQQHPGKAPKLMIYEGSKRPSGVSNRFSGSKSGNTASLTISGLQAEDEADYYCCSYAGSSTLIFGGGTKLTVL

SEQ ID NO: 180 - protein, 108 aa, synthetic construct
SYVLTQPPSVSVAPGQTASITCGGNNIGSKSVHWYQQKPGQAPVLVVYDDTDRPSGIPERFSGSNSGNTATLTISRVEAGDEADYYCQVWDSSSDHPVFGGGTKLTVL

SEQ ID NO: 181 - protein, 110 aa, synthetic construct
NFMLTQPHSVSESPGKTVTISCTRSSGSIASNYVQWYQQRPGSSPTPVIYEDYQRPSGVPDRFSGSIDSSSNSASLTISGLKTEDEADYYCQSFDRSNRVFGGGTKLTVL

SEQ ID NO: 182 - protein, 110 aa, synthetic construct; identical to SEQ ID NO: 178

SEQ ID NO: 183 - protein, 110 aa, synthetic construct
NFMLTQPHSVSESPGKTVTISCTRSSGNIARNYVQWYQQRPGSSPTTVIYEDNQRPSGVPDRFSGSIDSSSNSASLTISGLKTEDEADYYCQSYDSSNRVFGGGTKLTVL

SEQ ID NO: 184 - protein, 110 aa, synthetic construct
NFMLTQPHSVSESPGKTVTISCTRSSGSIASNFVQWYQQRPGSSPTTVIYEDNQRPSGVPDRFSGSIDSSSKSASLTISGLKTEDEADYYCQSYDSSNRVFGGGTKLTVL

SEQ ID NO: 185 - protein, 108 aa, synthetic construct
SYVLTQPPSVSVAPGQTARITCGGNNIGSKSVHWYQQKPGQAPVLVVYDDSDRPSGIPERFSGSNSENTATLTISRVEAGDEADYYCQVWDSTSDHPVFGGGTKLTVL

SEQ ID NO: 186 - protein, 110 aa, synthetic construct; identical to SEQ ID NO: 176

SEQ ID NO: 187 - protein, 111 aa, synthetic construct
NFMLTQPHSVSESPGKTVTFSCTRSSGSIASNYVQWYQQRPGSSPTTVIYVDNQRPSGVPDRFSGSIDSSSNSASLTISGLKTEDEADYYCQSYDSSNHWVFGGGTKLTVL

SEQ ID NO: 188 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGGGTKLTVL

SEQ ID NO: 189 - protein, 110 aa, synthetic construct
NVMLTQTHSVSESPGKTVTISCTRSSGSIASNYVQWYQQRPGSSPTTVIYEDNQRPSGVPDRFSGSIDSSSNSASLTISGLKTEDEADYYCQSYDSSNRVFGGGTKLTVL

SEQ ID NO: 190 - protein, 110 aa, synthetic construct
NFMLTQPHSVSESPGKTVTISCTRSSGSIASNFVQWYQQRPGSSPTTVIYEDNQRPSGVPDRFSGSIDSSSNSASLTISGLKTEDEADYYCQSYDSSNRVFGGGTKLTVL

SEQ ID NO: 191 - protein, 110 aa, synthetic construct; identical to SEQ ID NO: 188

SEQ ID NO: 192 - protein, 110 aa, synthetic construct
QSVLTQPPSVSAAPGQKVTISCSGSSSNIGNNYVSWYQQLPGTAPKLLIYDNYKRPSGIPDRFSGSKSGTSATLGITGLQTGDEADYYCGTWDSSLSAVVFGGGTKLTVL

SEQ ID NO: 193 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADTSGTYRVFGGGTKLTVL

SEQ ID NO: 194 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNVVFGGGTKLTVL

SEQ ID NO: 195 - protein, 110 aa, synthetic construct; identical to SEQ ID NO: 194

SEQ ID NO: 196 - protein, 110 aa, synthetic construct; identical to SEQ ID NO: 188

SEQ ID NO: 197 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGGGTKLTVL

SEQ ID NO: 198 - protein, 110 aa, synthetic construct
NFMLTQPHSVSESPGKTVTISCTRSSGNIASNYVQWYQQRPGSSPTTVIYEDNQRPSGVPDRFSGSIDSSSNSASLTISGLKTEDEADYYCQSYDSSNVVFGGGTKLTVL

SEQ ID NO: 199 - protein, 110 aa, synthetic construct
NFMLTQPHSVSESPGKTVTISCTRSSGSIANNFVQWFQQRPGSSPTDVIYEDNQRPSGVPDRFSGSIDSSSNSASLTISGLKTEDEADYYCQSYDSSNRVFGGGTKLTVL

SEQ ID NO: 200 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYSCQSYNGNNRVFGGGTKLTVL

SEQ ID NO: 201 - protein, 110 aa, synthetic construct
NFMLTQPHSVSESPGKTVTISCTRSSGSIASNYVQWYQQRPGSSPTPVIYEDNQRPSGVPDRFSGSIDSSSNSASLTISGLKTEDEADYFCQSYDNSNRVFGGGTKLTVL

SEQ ID NO: 202 - protein, 110 aa, synthetic construct
NFMLTQPHSVSESPGKTVTISCTRSSGNIASNYVQWYQQRPGSSPTAVIYEDNQRPSGVPDRFSGSIDSSSNSASLTISGLKTEDEADYYCQSYDGSNRVFGGGTKLTVL

SEQ ID NO: 203 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDNSNRVFGGGTKLTVL

SEQ ID NO: 204 - protein, 110 aa, synthetic construct; identical to SEQ ID NO: 188

SEQ ID NO: 205 - protein, 110 aa, synthetic construct
NFMLTQPHSVSESPGKTVTISCTRSSGSIASNYVQWYQQRPGSSPTTVIYEDYQRPSGVPARFSGSIDSSSNSASLTISGLKTEDEADYYCQSYDNNNRVFGGGTMLTVL

SEQ ID NO: 206 - protein, 110 aa, synthetic construct
DFMLTQPHSVSESPGKTVTISCTRSSGSIASNYVQWYQQRPGSSPTAVIYEDNQRPSGVPDRFSGSIDSSSNSASLTISGLKTEDEADYYCQSYDNSNRVFGGGTKLTVL

SEQ ID NO: 207 - protein, 110 aa, synthetic construct
NFMLTQPHSVSESPGKTVTISCTRSSGSIASNYVQWYQQRPGSSPTTVIYEDYQRPSGVPDRFSGSIDSSSNSASLTISGLKTEDEADYSCQSYDGNNRVFGGGTKLTVL

SEQ ID NO: 208 - protein, 111 aa, synthetic construct
NFMLTQPHSVSESPGKTVTISCTRSSGSIASNYVQWYQQRPGSSPTTVIYDDNQRPSGVPDRFSGSIDSSSNSASLTISGLKTEDEADYYCQSYDSSNHWVFGGGTKLTVL

SEQ ID NO: 209 - protein, 111 aa, synthetic construct
NFMLTQPHSVSESPGKTVTISCTRSSGSIASNYVQWYQQRPGSSPTTVIYDDNQRPSGVPDRFSGSIDSSSNSASLTISGLKTEDEAEYYCQSYDSSNHWVFGGGTKLTVL

SEQ ID NO: 210 - protein, 110 aa, synthetic construct
NFMLTQPHSVSESPGKTVTISCTRSSGSIASNYVQWFQQRPGSSPTTVIYDDNQRPSGVPDRFSGSIDSSSNSASLTISRLKTEDEADYYCQSYDSSNWVFGGGTKLTVL

SEQ ID NO: 211 - protein, 110 aa, synthetic construct; identical to SEQ ID NO: 188

SEQ ID NO: 212 - protein, 111 aa, synthetic construct
NFMLTQPHSVSESPGKTVTISCTRSSGSIASNYVQWYQQRPGSSPTTVIYDDNQRPSGVPDRFSGSIDSSSNSASLTISGLKTEDEADYYCQSYDSNNHWVFGGGTKLTVL

SEQ ID NO: 213 - protein, 111 aa, synthetic construct
NFMLTQPHSVSESPGKTVTISCTRSSGSIASNYVQWYQQRPGSSPTTVIYDDNQRPSGVPDRFSGSIDSSSSSASLTISGLKTEDEADYYCQSYDSSNHWVFGGGTKLTVL

SEQ ID NO: 214 - protein, 111 aa, synthetic construct
KLMLTQPHSVSESPGKTVTISCTRSSGSIARNYVQWYQQRPGSSPTTVIYDDNQRPSGVPDRFSGSIDSSSNSASLTISGLRTEDEADYYCQSYDSSDHWVFGGGTKLTVL

SEQ ID NO: 215 - protein, 110 aa, synthetic construct
NFMLTQPHSVSESPGKTVTISCTRSSGNIARNYVQWYQQRPGSSPTTVIYEDNQRPSGVPDRFSGSIDSSSNSASLTISGLKTEDEADYYCQSYDSGNWVFGGGAKLTVL

SEQ ID NO: 216 - protein, 110 aa, synthetic construct
NFMLTQPHSVSESPGKTVTISCTRSSGSIASNYVQWYQQRPGSSPTTVIYDDNQRPSGVPDRFSGSIDSSSNSASLTISGLKTEDEADYYCQSYDSNNWVFGGGTKLTVL

SEQ ID NO: 217 - protein, 110 aa, synthetic construct
NFMLTQPHSVSESPGKTITISCTRRNGNIASNYVQWYQQRPGRSPTTVIYEDNQRPSGVPDRFSGSIDSSSNSASLTISGLKTEDEADYYCQSYDSSNRVFGGGTKLTVL

SEQ ID NO: 218 - protein, 111 aa, synthetic construct
HFMLTQPHSVSESPGKTVTISCTRSSGSIASNYVQWYQQRPGSSPTTVIYDDNQRPSGVPDRFSGSIDSSSNSASLTISGLKTEDEADYYCQSYDSSNHWVFGGGTKLTVL

SEQ ID NO: 219 - protein, 111 aa, synthetic construct; identical to SEQ ID NO: 208

SEQ ID NO: 220 - protein, 111 aa, synthetic construct
NFMLTQPHSVSESPGKTVTISCTRSSGSIATNYVQWYQQRPGSSPTTLIYDDNQRPSGVPDRFSGSIDSSSNSASLTISGLKTEDEADYYCQSYDSSNHWVFGGGTKLTVL

SEQ ID NO: 221 - protein, 110 aa, synthetic construct; identical to SEQ ID NO: 188

SEQ ID NO: 222 - protein, 110 aa, synthetic construct
NFMLTQPHSVSESPGKTVTISCTRSSGSIASNYVQWYQQRPGSSPTTVIYEDSQRPSGVPDRFSGSIDSSSNSASLTISGLKTEDEADYYCQSYDSSNRVFGGGTKVTVL

SEQ ID NO: 223 - protein, 110 aa, synthetic construct
NFMLTQPHSVSESPGKTVTISCTRSSGSIASNYVQWFQQRPGSSPTTVIFEDNQRPSGVPDRFSGSIDSSSNSASLTISGLKTEDEADYYCQSYDSSVRVFGGGTKLTVL

SEQ ID NO: 224 - protein, 110 aa, synthetic construct
NFVLPQPHSVSESPGKTVTISCTRSSGSIASNYVQWYQQRPGSSPTSVIYDDSQRPSGVPDRFSGSIDSSSNSASLTISGLTTEDEADYYCQSYDSSNWVFGGGTKLTVL

SEQ ID NO: 225 - protein, 111 aa, synthetic construct
NFMLTQPHSVSESPGKTVTISCTRSSGSIASNYVQWYQQRPGSSPTTVIYDDNQRPSGVPDRFSGSIDSSSNSASLTISGLKTEDEADYYCQSYDSSNHWVFGEGTKLTVL

SEQ ID NO: 226 - protein, 110 aa, synthetic construct
NFMLTQPHSVSESPGKTVTISCTRSSGSIASNYVQWYQLRPGSSPTTVIYEDNQRPSGVPDRFSGSIDSSSNSASLTISGLKTEDEADYYCQSFDSSNRVFGGGTKLTVL

SEQ ID NO: 227 - protein, 110 aa, synthetic construct
NFMLTQPHSVSESPGKTVTISCTRSSGSVASNYVQWYQQRPGSSPTTVIYEDYQRPSGVPDRFSGSIDSSSNSASLTISGLKTEDEADYYCQSYDRSNRVFGGGTKLTVL

SEQ ID NO: 228 - protein, 111 aa, synthetic construct
YFMLTQPHSVSESPGKTVTISCTRSSGSIASNYVQWYQQRPGSSPTTVIYDDNQRPSGVPDRFSGSIDSSSNSASLTISGLKTEDEADYYCQSYDSSNHWVFGGGTKLTVL

SEQ ID NO: 229 - protein, 111 aa, synthetic construct; identical to SEQ ID NO: 208

SEQ ID NO: 230 - protein, 111 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNHWVFGGGTKLTVL

SEQ ID NO: 231 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDNSNRVFAGGTKLTVL

SEQ ID NO: 232 - protein, 110 aa, synthetic construct; identical to SEQ ID NO: 188

SEQ ID NO: 233 - protein, 110 aa, synthetic construct
NFMLTQPHSVSESPGKTVTISCTRSSGSIASNYVQWYQQRPGCSPTTVIYEDNQRPSGVPDRFSGSIDSSSNSASLTISGLKTEDEADYYCQSYDSNNRVFGGGTKLTVL

SEQ ID NO: 234 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 235 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0287181 A1
(43) Pub. Date: Aug. 29, 2024

(54) ANTI-CD3 ANTIBODIES AND METHODS FOR THEIR USE

(71) Applicant: ADANATE, INC., San Mateo, CA (US)

(72) Inventors: Raymond Shellooe, San Mateo, CA (US); James Sissons, San Mateo, CA (US); Allen Ebens, San Mateo, CA (US)

(21) Appl. No.: 18/584,421

(22) Filed: Feb. 22, 2024

Related U.S. Application Data
(60) Provisional application No. 63/447,755, filed on Feb. 23, 2023.

(51) Int. Cl. C07K 16/28

(52) U.S. Cl. CPC C07K 16/2809; C07K 2317/33

(57) ABSTRACT

The present invention relates to novel anti-CD3 antibodies (as monoclonal antibodies or as used in other formats, such as bispecific or multi-specific formats) and compositions comprising such antibodies or cells activated by such antibodies for use in treating disorders associated with CD3, such as human cancer therapy.

### FIELD OF THE INVENTION

The present invention relates to novel anti-CD3 antibodies (as monoclonal antibodies or as used in other formats, such as bispecific or multi-specific formats) and compositions comprising such antibodies or cells activated by such antibodies for use in treating disorders associated with CD3, such as human cancer therapy.

### SEQUENCE LISTING

This application contains a sequence listing which has been submitted in the XML format and is hereby incorporated by reference in its entirety. The sequence listing file created on Feb. 22, 2024, is named ADAN-001-01US.xml, and is 216 KB in size.

### BACKGROUND OF THE INVENTION

The body s immune system serves as a defense against a variety of conditions, including, e.g, injury, infection and neoplasia, and is mediated by two separate but interrelated systems: the cellular and humoral immune systems. Generally speaking, the humoral system is mediated by soluble products (antibodies or immunoglobulins) that have the ability to combine with and neutralize products recognized by the system as being foreign to the body. In contrast, the cellular immune system involves the mobilization of certain cells, termed T cells, that serve a variety of therapeutic roles. T cells are lymphocytes that are derived from the thymus and circulate between the tissues, lymphatic system and the circulatory system. They act against, or in response to, a variety of foreign structures (antigens). In many instances these foreign antigens are expressed on host cells as a result of neoplasia or infection. Although T cells do not themselves secrete antibodies, they are usually required for antibody secretion by the second class of lymphocytes, B cells (which derive from bone marrow). Critically, T cells exhibit extraordinary immunological specificity so as to be capable of discerning one antigen from another).

A naive T cell, e.g., a T cell which has not yet encountered its specific antigen, is activated when it first encounters a specific peptide:MHC complex on an antigen presenting cell. The antigen presenting cell may be a B cell, a macrophage or a dendritic cell. When a naive T cell encounters a specific peptide:MHC complex on an antigen presenting cell, a signal is delivered through the T-cell receptor which induces a change in the conformation of the T cell's lymphocyte function associated antigen (LFA) molecules, and increases their affinity for intercellular adhesion molecules (ICAMs) present on the surface of the antigen presenting cell. The signal generated by the interaction of the T cell with an antigen presenting cell is necessary, but not sufficient, to activate a naive T cell. A second co-stimulatory signal is required. The naive T cell can be activated only by an antigen-presenting cell carrying both a specific peptide MHC complex and a co-stimulatory molecule on its surface. Antigen recognition by a naive T cell in the absence of co-stimulation results in the T cell becoming anergic. The need for two signals to activate T cells and B cells such that they achieve an adaptive immune response may provide a mechanism for avoiding responses to self-antigens that may be present on an antigen presenting cell at locations in the system where it can be recognized by a T cell. Where contact of a T cell with an antigen presenting cell results in the generation of only one of two required signals, the T cell does not become activated and an adaptive immune response does not occur.

The efficiency with which humans and other mammals develop an immunological response to pathogens and foreign substances rests on two characteristics: the exquisite specificity of the immune response for antigen recognition, and the immunological memory that allows for faster and more vigorous responses upon re-activation with the same antigen (Portolés, P. et al. (2009) “The TCR/CD3 Complex: Opening the Gate to Successful Vaccination,” Current Pharmaceutical Design 15:3290-3300; Guy, C. S. et al. (2009) “Organization of Proximal Signal Initiation at the TCR: CD3 Complex,” Immunol Rev. 232(1): 7-21). The specificity of the response of T-cells is mediated by the recognition of antigen (displayed on Antigen-Presenting Cells (APCs) by a molecular complex involving the T Cell Receptor (“TCR”) and the cell surface receptor ligand, CD3. The TCR is a covalently linked heterodimer of α and β chains (“TCRαβ”). These chains are class I membrane polypeptides of 259 (α) and 296 (β) amino acids in length. The CD3 molecule is a complex containing a CD3 γ chain, a CD3 δ chain, and two CD3 ε chains associated as three dimers (εγ, εδ, ζζ) (Guy, C. S. et al. (2009) “Organization of Proximal Signal Initiation at the TOR: CD3 Complex,” Immunol Rev. 232(1): 7-21; Call, M. E. et al. (2007) “Common Themes In The Assembly And Architecture Of Activating Immune Receptors,” Nat. Rev. Immunol. 7:841-850; Weiss, A. (1993) “T Cell Antigen Receptor Signal Transduction: A Tale Of Tails And Cytoplasmic Protein-Tyrosine Kinases,” Cell 73:209-212). The TCR and CD3 complex, along with the CD3 ζ chain zeta chain (also known as T-cell receptor T3 zeta chain or CD247) comprise the TCR complex (van der Merwe, P. A. etc. (epub Dec. 3, 2010) “Mechanisms For T Cell Receptor Triggering,” Nat. Rev. Immunol. 11:47-55; Wucherpfennig, K. W. et al. (2010) “Structural Biology of the T-cell Receptor: Insights into Receptor Assembly, Ligand Recognition, and Initiation of Signaling,” Cold Spring Harb. Perspect. Biol. 2:a005140). The complex is particularly significant since it contains a large number (ten) of immunoreceptor tyrosine-based activation motifs (ITAMs).

In mature T cells, TCR/CD3 activation by foreign antigenic peptides associated to self-MHC molecules is the first step needed for the expansion of antigen-specific T cells, and their differentiation into effector or memory T lymphocytes. These processes involve the phosphorylation of the immunoreceptor tyrosine-based activation motifs (ITAMs) of the TCR complex. Because the TCR complex has such a large number of ITAMS (10 in all), and these ITAMS are arrayed in tandem (due to the dimerization of the constituent chains), phosphorylation of the relevant tyrosine residues upon TCR ligation creates paired docking sites for proteins that contain Src homology 2 (SH2) domains such as the & chain-associated protein of 70 kDa (ZAP-70), and thereby initiate an amplifying signaling cascade which leads to T-cell activation and differentiation (Guy, C. S. et al. (2009) “Organization of Proximal Signal Initiation at the TCR: CD3 Complex,” Immunol Rev. 232(1):7-21).

The outcome of these processes is modulated by the intensity and quality of the antigen stimulus, as well as by the nature of accompanying signals delivered by co-receptor and co-stimulatory surface molecules, or by cytokine receptors (Portoles, P. et al. (2009) “The TCR/CD3 Complex: Opening the Gate to Successful Vaccination,” Current Pharmaceutical Design 15:3290-3300; Riha, P. et al. (2010) “CD28 (Co-Signaling In The Adaptive Immune Response,” Self/Nonself 1(3):231-240). Although TCR stimulation is a prerequisite for T-cell activation, it is well recognized that engagement of co-stimulatory molecules, such as CD28, is necessary for full T-cell activation and differentiation (Guy, C. S. et al. (2009) “Organization of Proximal Signal Initiation at the TCR:CD3 Complex,” Immunol Rev. 232(1): 7-21).

Due to the fundamental nature of CD3 in initiating an anti-antigen response, monoclonal antibodies against this receptor have been proposed as being capable of blocking or at least modulating the immune process and thus as agents for the treatment of inflammatory and/or autoimmune disease. Indeed, anti-CD3 antibodies were the first antibody approved for the human therapy (St. Clair E. W. (2009) “Novel Targeted Therapies for Autoimmunity,” Curr. Opin. Immunol. 21(6):648-657). Anti-CD3 antibody (marketed as ORTHOCLONE™ OKT3™ by Janssen-Cilag) has been administered to reduce acute rejection in patients with organ transplants and as a treatment for lymphoblastic leukemia (Cosimi, A. B. et al. (1981) “Use Of Monoclonal Antibodies To T-Cell Subsets For Immunologic Monitoring And Treatment In Recipients Of Renal Allografts,” N. Engl. J. Med. 305:308-314; Kung, P. et al. (1979) Monoclonal antibodies defining distinctive human T cell surface antigens,” Science 206:347-349; Vigeral, P. et al. (1986) “Prophylactic Use Of OK13 Monoclonal Antibody In Cadaver Kidney Recipients. Utilization Of OKT3 As The Sole Immunosuppressive Agent,” Transplantation 41:730-733; Midtvedt, K. et al. (2003) “Individualized T Cell Monitored Administration Of ATG Versus OKT3 In Steroid-Resistant Kidney Graft Rejection,” Clin. Transplant. 17(1):69-74; Gramatzki, M. et al. (1995) “Therapy With OK13 Monoclonal Antibody In Refractory T Cell Acute Lymphoblastic Leukemia Induces Interleukin-2 Responsiveness,” Leukemia 9(3): 382-390; Herold, K. C. et al. (2002) “Anti-CD3 Monoclonal Antibody In New-Onset Type 1 Diabetes Mellitus,” N. Engl. J. Med. 346:1692-1698; Cole, M. S. et al. (1997) “Human IgG2 Variants Of Chimeric Anti-CD3 Are Nonmitogenic to T cells,” J. Immunol. 159(7): 3613-3621; Cole, M. S. et al. (1999) “Hum291, A Humanized Anti-CD3 Antibody, Is Immunosuppressive To T Cells While Exhibiting Reduced Mitogenicity in vitro,” Transplantation 68:563-571; U.S. Pat. Nos. 6,491,916; 5,585,097 and 6,706,265).

However, such anti-CD3 treatment has not proven to be specific enough to avoid side effects (Ludvigsson, J. (2009) “The Role of Immunomodulation Therapy in Autoimmune Diabetes,” J. Diabetes Sci. Technol. 3(2):320-330). Repeated daily administration of OKT3 results in profound immunosuppression and provides effective treatment of rejection following renal transplantation. The in vivo administration of OKT3 results in both T cell activation and suppression of immune responses. However, the use of OKT3 has been hampered by a first toxic dose reaction syndrome that is related to initial T-cell activation events and to the ensuing release of cytokines that occurs before immunosuppression of T cell responses. The reported side effects that follow the first and sometimes the second injection of this mouse monoclonal antibody include a “flu-like” syndrome consisting of high fever, chills, headache, and gastrointestinal symptoms (vomiting and diarrhea) and in severe cases pulmonary edema within hours of treatment has been noted (Thistlethwaite, J. R. Jr. et al. (1988) “Complications and Monitoring of OKT3 Therapy,” Am. J. Kidney Dis. 11:112-119). This syndrome is believed to reflect OKT3-mediated cross-linking of the TCR/CD3 complex on the T cell surface and the resultant release of cytokines (e.g., tumor necrosis factor alpha (TNFα), interferon-γ, interleukins IL-2, IL-3, IL-4, IL-6, IL-10 and granulocyte-macrophage colony-stimulating factor (Masharani, U. B. et al. (2010) “Teplizumab Therapy For Type 1 Diabetes,” Expert Opin. Biol. Ther. 10(3):459-465; Abramowicz, D. et al. (1989) “Release Of Tumor Necrosis Factor, Interleukin-2, And Gamma-Interferon In Serum After Injection Of OKT3 Monoclonal Antibody In Kidney Transplant Recipients,” Transplantation 47:606-608; Ferran, C. et al. (1990) “Cytokine-Related Syndrome Following Injection Of Anti-CD3 Monoclonal Antibody: Further Evidence For Transient In Vivo T Cell Activation,” Eur. J. Immunol. 20:509-515; Hirsch, R. et al. (12989) “Effects Of In Vivo Administration Of Anti-CD3 Monoclonal Antibody On T Cell Function In Mice. II. In Vivo Activation Of T Cells,” J. Immunol. 142:737-743). The use of anti-CD3 antibodies is disclosed in U.S. Pat. Nos. 7,883,703; 7,728,114; 7,635,472; 7,575,923; and 7,381,903, and in United States Patent Publications Nos. 2010/0150918; 2010/0209437; 2010/0183554; 2010/0015142, 2008/0095766, 2007/0077246 and in PCT Publication WO2008/119567.

### BRIEF SUMMARY OF THE INVENTION

The present invention relates to novel anti-CD3 antibodies (as monoclonal antibodies or as used in other formats, such as bispecific or multi-specific formats) and compositions comprising such antibodies or cells activated by such antibodies for use in treating disorders associated with CD3, such as human cancer therapy. The present invention also includes compositions comprising one or more of these peptides/antibodies, or fragments thereof, and/or immune cells that are modified to include and/or be activated by one or more of these antibodies, or fragments thereof, to treat a disease or condition, such as cancer. Antibodies of the present invention may similarly find use as a targeting arm of a bispecific or multi-specific format.

In one aspect, the present invention provides an anti-CD3 antibody or antibody fragment comprising a heavy chain CDR3 sequence (VH) comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59. Additionally or alternatively, an anti-CD3 antibody or antibody fragment of the present invention may comprise a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

Additionally or alternatively, the present invention provides an anti-CD3 antibody or antibody fragment comprising a heavy chain CDR3 sequence (VH) comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59 and a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

Additionally or alternatively, an anti-CD3 antibody or antibody fragment of the present invention may comprise a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175. Additionally or alternatively, an anti-CD3 antibody or antibody fragment of the present invention may comprise a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

Additionally or alternatively, an anti-CD3 antibody or antibody fragment of the present invention may comprise a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175 and a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

In certain aspects, the present invention provides methods for treating a disorder, such as cancer, using one or more antibodies of the invention as described above, by, at least administering such antibodies to a subject, such as a human subject.

In certain aspects, the present invention provides immunoconjugates and/or compositions comprising such immunoconjugates, wherein said immunoconjugates comprise an anti-CD3 antibody of the invention conjugated to another therapeutic agent, such as an anti-cancer agent. The invention further provides immunoconjugates comprising two or more different anti-CD3 antibodies or fragment thereof, wherein each different anti-CD3 antibody or fragment targets a different CD3 fragment or epitope.

A further aspect of the invention relates to a nucleic acid molecule having a nucleotide sequence that encodes an anti-CD3 antibody or fragment thereof, as disclosed herein, as well as expression vectors comprising such a polynucleotide and host cells that have been transfected with such an expression vector.

Aspects of the invention also provide methods for producing the anti-CD3 antibodies, fragments thereof, and compositions of the invention.

The present invention also provides methods for treating a disease in a human or animal subject, in particular treatment of cancer in humans, by administering an anti-CD3 antibody or composition of the invention to said subject. The invention also includes the use of one or more anti-CD3 antibodies of the invention for preparation of a medicament for use in treating a disease in a human or animal, in particular for the treatment of cancer in humans.

In some embodiments, the antibodies herein are full length antibodies. In some embodiments, the antibodies are an IgA, an IgD, an IgE, an IgG, or an IgM antibody. In some embodiments, the anti-CD3 antibody is an IgG antibody (e.g., an IgG1, IgG2, or IgG3 antibody).

In some embodiments, the antibodies herein are an antibody fragment. In some embodiments, the antibodies are an Fv fragment, a Fab fragment, a F(ab′)₂ fragment, a Fab′ fragment, a Fab′-SH, an scFv (sFv) fragment, and an scFv-Fc fragment. In some embodiments, the bispecific antibody is an scFv fragment. In some embodiments, the antibodies herein are monoclonal, human, humanized, or chimeric.

In some embodiments, the antibodies further comprise an Fc region. In some embodiments, the antibodies comprise one or more heavy chain constant domains, wherein the one or more heavy chain constant domains are selected from a first CH1 domain, a first CH2 domain, a first CH3 domain, a second CH1 domain, a second CH2 domain, and a second CH3 domain. In some embodiments, one or more heavy constant chain domains are paired with another heavy chain constant domain.

In some embodiments, the antibodies further comprise a glycosylation site mutation. In some embodiments, the mutation reduces effector function. In some embodiments, the mutation is a substitution mutation.

### DETAILED DESCRIPTION OF THE INVENTION

The T cell receptor (TCR) binds to antigens (Ags) displayed by major histocompatibility complexes (MHCs) and plays critical roles in T cell function. But the TCR does not possess intracellular signaling by itself. Instead, TCR non-covalently associates with the Cluster of Differentiation 3 (CD3) complex and triggers intracellular signaling through immunoreceptor tyrosine-based activation motifs (ITAM) of CD3. The CD3 T cell co-receptor helps to activate both the cytotoxic T cell (CD8+ naive T cells) and also T helper cells (CD4+ naive T cells). It consists of a protein complex and is composed of four distinct chains. In mammals, the complex contains a CD3γ chain, a CD3δ chain, and two CD3ε chains. These chains associate with the T cell receptor (TCR) and the CD3ζ chain (zeta-chain) to generate an activation signal in T lymphocytes. The TCR, ζ-chain, and CD3γ, δ, and ε chains together constitute the TCR complex. The CD3 four-chain complex then forms CD3εγ, CD3εδ and δδ dimers in 1:1:1 stoichiometry.

CD3 is initially expressed in the cytoplasm of pro-thymocytes, the stem cells from which T cells arise in the thymus. The pro-thymocytes differentiate into common thymocytes, and then into medullary thymocytes, and it is at this latter stage that CD3 antigen begins to migrate to the cell membrane. The antigen is found bound to the membranes of all mature T cells, and in virtually no other cell type, although it does appear to be present in small amounts in Purkinje cells.

This high specificity, combined with the presence of CD3 at all stages of T cell development, makes it a useful immunohistochemical marker for T cells in tissue sections. The antigen remains present in almost all T cell lymphomas and leukemias, and can therefore be used to distinguish them from superficially similar B cell and myeloid neoplasms. Some antibodies against CD3ε chain have been shown to activate TCR-CD3 complex, possibly by clustering CD3 complex on T cells. In addition, bispecific antibodies targeting both CD3 and a tumor specific antigen have been studied for redirected tumor eradication by T cells. Because CD3 is required for T cell activation, drugs (often monoclonal antibodies) that target it are being investigated as immunosuppressant therapies (e.g., otelixizumab) for type 1 diabetes and other autoimmune diseases.

The present invention is directed to novel peptides (e.g., antibodies and antibody fragments) that bind to CD3. The present invention also includes compositions comprising one or more of these peptides/antibodies, or fragments thereof, and/or immune cells that are modified to include and/or be activated by one or more of these antibodies, or fragments thereof, to treat a disease or condition, such as cancer.

The presently disclosed antibodies may provide treatments that are far more effective than current therapies than present CD3 treatments. The presently disclosed anti-CD3 antibodies of the invention may be included as part of a treatment regime, which may include, for example, providing two or more such antibodies, and/or in combination with other treatments such as chemotherapy.

### Definitions

The term “antibody” or “antibody molecule” describes a functional component of serum and is often referred to either as a collection of molecules (antibodies or immunoglobulin) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An antibody is generally considered as monospecific, and a composition of antibodies may be monoclonal (i.e., consisting of identical antibody molecules) or polyclonal (i.e., a plurality of different antibodies that may react with the same or different epitopes on the same antigen or on distinct/different antigens). An antibody has a unique structure enabling it to bind specifically to its corresponding antigen, and all natural antibodies have the same overall basic structure of two identical light chains and two identical heavy chains.

As used herein, “antibody” or “antibodies” may include chimeric and single chain antibodies, as binding fragments of antibodies, such as Fab, Fv fragments or single chain Fv (scFv) fragments, and multimeric forms, e.g., dimeric IgA molecules or pentavalent IgM. Antibodies of the invention may be of human or non-human origin, for example a murine or other rodent-derived antibody, or a chimeric, humanized or reshaped antibody based e.g., on a murine antibody.

A heavy chain of an antibody typically includes a heavy chain variable region (VH) and a heavy chain constant region. The heavy chain constant region usually comprises three domains, referred to as CH1, CH2 and CH3. An antibody light chain includes a light chain variable region (VL) and a light chain constant region. The light chain constant region includes a single domain, referred to as CL. The VH and VL regions are subdivided into regions of hypervariability (“hypervariable regions”), which may be hypervariable in sequence and/or in looped structure. These regions are also referred to as complementarity determining regions (CDRs), which are interspersed with regions that are more conserved, termed framework regions (FRs). Each VH and VL typically includes three CDRs and four FRs, arranged from the amino terminus to the carboxy terminus in the following order: FR1, CDR1, FR2, CDR2, FR3, CDR3, FR4. Amino acid residues in the variable regions are often numbered using a standardized numbering method known as the Kabat numbering scheme (Kabat et al. (1991) Sequences of Proteins of Immunological Interest, 5th Ed. Public Health Service, National Institutes of Health, Bethesda, Md., USA).

The antibody identifiers found in the tables of this application, e.g., “70701_06 A03A”, refer to the specific antibodies.

As used herein, an antibody or fragment “derived from” or “based on” an antibody means that the “derived” antibody comprises, depending on the particular context, one of the following: the heavy chain CDR3 sequence of said specified antibody; the heavy chain CDR3 sequence and the light chain CDR3 sequence of said specified antibody; the heavy chain CDR1, CDR2 and CDR3 sequences and light chain CDR1, CDR2 and CDR3 sequences of said specified antibody; or the heavy chain variable region sequence and the light chain variable region sequence of said specified antibody, or a humanized variant of said heavy chain variable region sequence and/or light chain variable region sequence, or a heavy chain and/or light chain variable region sequence having at least 80%, 85%, 90% or 95% sequence identity, such as at least 96%, 97%, 98% or 99% sequence identity, with the respective heavy chain and light chain variable region sequences.

An antibody that is derived from or based on a specified antibody described herein will generally bind the same CD3 epitope as said specified antibody and will preferably exhibit substantially the same activity as said specified antibody.

The specificity of an antibody [symbol] interaction with a target antigen is driven, primarily, by the amino acid residues located in the six CDRs of the heavy and light chain. The amino acid sequences within CDRs are more variable between individual antibodies than sequences outside of CDRs. Since CDR sequences are responsible for most antibody-antigen interactions, antibodies that mimic the properties of a specific naturally occurring antibody, or any specific antibody with a given amino acid sequence, can be expressed by constructing expression vectors that express CDR sequences from the specific antibody grafted into framework sequences from a different antibody. This permits “humanization” of a non-human antibody, which will nonetheless substantially maintain the binding specificity and affinity of the original antibody. Nonetheless, in preferred aspects, the anti-CD3 antibodies are human antibodies.

A “chimeric antibody” means an antibody that includes one or more regions from one antibody and one or more regions from one or more different antibodies. A “chimeric antibody” is typically an antibody that is partially of human origin and partially of non-human origin. Chimeric antibodies may be preferred over non-human antibodies, as they have been shown to reduce the risk of a human anti-antibody response. A chimeric antibody may include an antibody in which the variable region sequences are murine sequences derived from immunization of a mouse, while the constant region sequences are human. In the case of a chimeric antibody, the non-human parts, which often include the framework regions of the variable region sequences, may be further altered in order to humanize the antibody.

In preferred aspects, the presently disclosed antibodies of the invention are derived from transgenic mice that contain human antibody gene segments, such that the antibodies are human antibodies derived by hybridoma technology from transgenic mice.

The terms “heavy chain variable region sequence” and “light chain variable region sequence” and similar terms as used herein with reference to any specific amino acid sequence encompass not only that specific sequence, but also any recombinant antibodies, human antibodies, including those derived from transgenic mice that contain human antibody gene segments, such that the antibodies are human antibodies derived by hybridoma technology, and/or humanized variants thereof.

As used herein, a reference to a heavy chain variable region sequence or a light chain variable region sequence with a particular minimum level of sequence identity compared to a specified heavy chain or light chain variable region sequence.

A “recombinant antibody” is an antibody that is expressed from a cell or cell line transfected with an expression vector (or possibly more than one expression vector, typically two expression vectors) comprising the coding sequence of the antibody, where said coding sequence is not naturally associated with the cell.

A “vector” is a nucleic acid molecule into which a nucleic acid sequence can be inserted for transport between different genetic environments and/or for expression in a host cell. A vector that carries regulatory elements for transcription of the nucleic acid sequence (at least a suitable promoter) is referred to as an “an expression vector”. The terms “plasmid” and “vector” may be used interchangeably. Expression vectors used in the context of the present invention may be of any suitable type known in the art, for example, a viral vectors or plasmids.

It is well-known in the art that antibodies exist as different isotypes, such as the human isotypes IgG1, IgG2, IgG3, IgG4, IgA1 and IgA2, or the murine isotypes IgG1, IgG2a, IgG2b, IgG3 and IgA. An antibody of the invention may be of any isotype.

In certain aspects, compositions of the invention include antibody compositions comprising a plurality of individual anti-CD3 antibodies, e.g., 2, 3, 4, 5, 6, 7, 8, 9, 10, or 10 or more different CD3 antibodies.

A “CDR” or “complementarity determining region” refers to the “hypervariable” regions found in the variable domains of an antibody that are primarily responsible for determining the antibody s binding specificity. Each of the heavy and light chains of an antibody contain three CDR regions, referred to as CDR1, CDR2 and CDR3, of which CDR3 shows the greatest variability.

An “epitope” describes a part of a larger molecule (e.g., antigen or antigenic site) having antigenic or immunogenic activity in an animal. An epitope having immunogenic activity is a portion of a larger molecule that elicits an antibody response. An epitope having antigenic activity is a portion of a larger molecule to which an antibody immune-specifically binds. Antigenic epitopes are not necessarily immunogenic. An antigen is a substance to which an antibody or antibody fragment specifically binds, such as a toxin, virus, bacteria, protein or DNA. An antigen or antigenic site may have more than one epitope and may be capable of stimulating an immune response.

Epitopes may be linear or conformational. A linear epitope generally consists of about 6 to 10 adjacent amino acids on a protein molecule that are recognized by an antibody. In contrast, a conformational epitope consists of amino acids that are not arranged sequentially, but where an antibody recognizes a particular three-dimensional structure. When a protein molecule folds into a three-dimensional structure, the amino acids forming the epitope are juxtaposed, enabling the antibody to recognize the conformational epitope. In a denatured protein, only linear epitopes are recognized. A conformational epitope, by definition, must be on the outside of the folded protein.

The term “distinct epitopes” refers to the fact that when two different antibodies of the invention bind distinct epitopes, there is less than 100% competition for antigen binding, preferably less than 80% competition for antigen binding, more preferably less than 50% competition for antigen binding, and most preferably as little competition as possible, such as less than about 25% competition for antigen binding.

Antibodies capable of competing with each other for binding to the same antigen may bind the same or overlapping epitopes or may have a binding site in the close vicinity of one another, so that competition is mainly caused by steric hindrance. An analysis for “distinct epitopes” of antibody pairs may be performed by methods known in the art, for example by way of binding experiments under saturating antibody conditions using either FACS (fluorescence activated cell sorting) or other flow cytometry analysis on cells expressing CD3 and individual fluorescent labeled antibodies, or by Surface Plasmon Resonance (SPR) using CD3 antigens attached to a flow cell surface.

Antibodies binding to different epitopes on the same antigen can have varying effects on the activity of the antigen to which they bind, depending on the location of the epitope. An antibody binding to an epitope in an active site of the antigen may block the function of the antigen completely, whereas another antibody binding at a different epitope may have no or little effect on the activity of the antigen alone. Such antibodies may, however, still activate complement and thereby result in the elimination of the antigen, and may result in synergistic effects when combined with one or more antibodies binding at different epitopes on the same antigen.

“Immunoglobulin” is a collective designation of the mixture of antibodies found in blood or serum, but may also be used to designate a mixture of antibodies derived from other sources.

The term “cognate V_{H} and V_{L} coding pair” describes an original pair of V_{H} and V_{L} coding sequences contained within or derived from the same antibody-producing cell. Thus, a cognate V_{H} and V_{L} pair represents the V_{H} and V_{L} pairing originally present in the donor from which such a cell is derived. The term “an antibody expressed from a V_{H} and V_{L} coding pair” indicates that an antibody or an antibody fragment is produced from a vector, plasmid or other polynucleotide containing the V_{H} and V_{L} coding sequence. When a cognate V_{H} and V_{L} coding pair is expressed, either as a complete antibody or as a stable fragment thereof, they preserve the binding affinity and specificity of the antibody originally expressed from the cell they are derived from. A library of cognate pairs is also termed a repertoire or collection of cognate pairs, and may be kept individually or pooled.

By “protein” or “polypeptide” is meant any chain of amino acids, regardless of length or post-translational modification. Proteins can exist as monomers or multimers, comprising two or more assembled polypeptide chains, fragments of proteins, polypeptides, oligopeptides, or peptides.

The term “head-to-head promoters” refers to a promoter pair being placed in close proximity so that transcription of two gene fragments driven by the promoters occurs in opposite directions. Head-to-head promoters are also known as bi-directional promoters.

The term “transfection” is herein used as a broad term for introducing foreign DNA into a cell. The term is also meant to cover other functional equivalent methods for introducing foreign DNA into a cell, such as e.g., transformation, infection, transduction or fusion of a donor cell and an acceptor cell.

As used herein, CD3 is intended to include variants, isoforms and species homologs of CD3. Preferably, binding of an antibody of the invention to CD3 inhibits the growth of cells expressing CD3. In certain aspects, this inhabitation is caused by inhibiting formation of heteromeric complexes between CD3 and other ErbB family members.

As used herein, the term “inhibits growth” (e.g., referring to cells) is intended to include any measurable decrease in the proliferation (increase in number of cells) or metabolism of a cell when contacted with an anti-CD3 antibody as compared to the growth of the same cells in the absence of an anti-CD3 antibody, e.g., inhibition of growth of a cell culture by at least about 10%, and preferably more, such as at least about 20% or 30%, more preferably at least about 40% or 50%, such as at least about 60%, 70%, 80%, 90%, 99% or even 100%.

The term “treatment” as used herein refers to administration of an anti CD3 antibody, antibody composition of the invention, or composition of immune cells that express or are activated by a CD3 antibody or fragment thereof, in a sufficient amount to ease, reduce, ameliorate or eradicate (cure) symptoms or disease states.

The percent identity between two sequences, e.g., variable region sequences, refers to the number of identical positions shared by the sequences (calculated as # of identical positions/total # of positions×100), taking into account gaps that must be introduced for optimal alignment of the two sequences. The comparison of sequences and determination of percent identity between two sequences may be accomplished using readily available software. Suitable software programs are available from various sources, both for online use and for download, and for alignment of both protein and nucleotide sequences. One suitable program is ClustalW (Thompson et al. (1994) Nucleic Acids Res. 11; 22(22):4673-80), available from www.clustal.org.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

An “affinity matured” antibody refers to an antibody with one or more alterations in one or more hypervariable regions, compared to a parent antibody, which does not possess such alterations, such alterations resulting in an improvement in the affinity of the antibody for antigen.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA).

In certain embodiments, an antibody that binds to CD3 has a dissociation constant (Kd) of ≤1 μM, ≤100 nM, ≤10 nM, ≤1 nM, ≤0.1 nM, ≤0.01 nM, or ≤0.001 nM (e.g., 10⁻⁶M or less, e.g., from 10⁻⁸ M to 10⁻¹³ M, e.g., from 10⁻⁹ M to 10⁻¹³ M). In preferred aspects, this affinity range is the “optimal affinity range”, which retains anti-tumor activity but has reduced toxicity due to reduced cytokine release. In certain preferred aspects, the anti-CD3 antibody has an affinity in the range of 30 nM to 40 nM. In preferred aspects, the anti-CD3 antibody has an affinity in the range of 30-40 nM as measured by alanine scanning of the HC CDR3 of the antibody.

In certain embodiments, an anti-CD3 antibody binds to an epitope of CD3 that is conserved among CD3s from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP-000724), which is 207 amino acids in length, and human CD3γ protein (NCBI RefSeq No. NP-000064), which is 182 amino acids in length.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Effector functions” refer to those biological activities attributable to the Fc region of an antibody, which vary with the antibody isotype. Examples of antibody effector functions include: C1 q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of a compound, for example, an anti-CD3 antibody of the invention or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region residues. The FR of a variable domain generally consists of four FR domains: FR1, FR2, FR3, and FR4. Accordingly, the HVR and FR sequences generally appear in the following sequence in VH (or VL): FR1-H1(L)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol. 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

For example, in preferred aspects, technologies such as AlivaMab Mouse technology are used to produce the anti-CD3 antibodies described herein. Generally, AlivaMab Mouse technology is used to generate panels of monoclonal antibodies (mAbs) against a human antigen of interest, such as those expressed by or associated with tumor cells, e.g., variants of HER2.

The AlivaMab Mouse is a transgenic mouse that produces chimeric human-mouse monoclonal antibodies comprising fully human Fab and upper hinge regions and mouse middle hinge and Fc regions. Optimized constant domains facilitate the generation and identification of antibodies that retain structure-function characteristics. Antibodies produced using AlivaMab Mouse technology possess biophysical properties, which are predictive and comparable to that of fully human antibody counterparts.

Antibodies produced by AlivaMab Kappa Mice include a chimeric immunoglobulin heavy (IgH) chain and a human immunoglobulin kappa (IgK) light chain. Antibodies produced by AlivaMab Lambda Mice include a chimeric IgH chain and a human immunoglobulin lambda (IgK) light chain. The chimeric IgH chain of the AlivaMab Mouse antibodies include a human variable region comprising a human variable heavy (V_{H}) domain, a human diversity heavy (DH) domain, and a human joining heavy (JH) domain, a human constant heavy 1 (CH1) domain, a human upper hinge region (except for Ou, which is naturally missing an upper hinge region), a mouse middle hinge region, a mouse CH2 domain, and a mouse CH3 domain.

When a lead candidate antibody is discovered, the human heavy chain variable region is readily appended to a fully human constant region while maintaining the antigen-binding characteristics of the parent chimeric antibody that were developed in vivo in the AlivaMab Mouse. In one embodiment, the human heavy chain variable region, CH1 and, optionally, upper hinge region of the chimeric antibody are appended to human hinge, a human CH2 domain and a human CH3 domain in order to produce a fully human antibody.

Portions of variable regions from the antibodies produced from AlivaMab Mouse technology may include all or a combination of the complementarity determining regions (CDRs) of the VH and/or VL. The variable regions may be formatted with constant regions, either native or modified for various desired effector functions, in a standard antibody structure (two heavy chains with two light chains). The variable regions may also be formatted as multi-specific antibodies, e.g., bispecific antibodies binding to two different epitopes or to two different antigens. The variable regions may also be formatted as antibody fragments, e.g., single-domain antibodies comprising a single VH or VL, Fabs or Fab′2. The antibodies may also be used as antibody-drug conjugates, or carry other additions such as small molecule toxins, biologic toxins, cytokines, oligopeptides, or RNAs to increase therapeutic modality and/or increase safety.

Methods for producing the anti-CDR3 antibodies of the invention using AlivaMab mouse technology may include immunizing AlivaMab Kappa Mice and AlivaMab Lambda Mice with an antigen of interest. Generally, within two weeks, the mice are sacrificed and terminal materials collected. Spleens and lymph nodes may be prepared and fused with myeloma cells (such as CRL-2016 cells) using a PEG based method as generally described in “Antibodies: A Laboratory Manual” (Harlow and Lane 1988 CSH Press) to establish hybridomas.

Hybridomas may be grown in 384-well tissue culture plates and supernatants from individual wells were screened by ELISA for production of antibodies recognizing the antigen of interest. Positive wells are then transferred to 48-well plates, expanded, and supernatants were collected for antigen binding confirmation by ELISA. Positive supernatants may also be counter-screened against a non-related histidine-tagged protein. Hybridoma lines each from AlivaMab Kappa Mice and AlivaMab Lambda Mice are confirmed to bind to the antigen specifically by ELISA and are picked at random and single-cell cloned into 96-well plates. They are grown into colonies and the supernatant from these individual colonies is screened by ELISA to re-confirm monoclonal antibody binding to the antigen of interest. These supernatants are then screened by FACS to confirm binding to the native antigen expressed on cells.

AlivaMab Mouse technology and methods for producing antibodies using such technologies can be found in WO 2010/039900 and WO 2011/123708, which are incorporated herein in their entirety.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

To “humanize” an antibody means that an antibody of wholly or partially of non-human origin, for example a murine antibody obtained from immunization of mice with an antigen of interest or a chimeric antibody based on such a murine antibody, can have amino acids replaced, particularly n the framework regions and constant domains of the heavy and light chains, to avoid or minimize an immune response in humans. It is known that all antibodies have the potential for eliciting a human anti-antibody response, which correlates to some extent with the degree of “humanness” of the antibody in question.

Non-human antibodies tend to be more immunogenic than human antibodies. Chimeric antibodies, where the foreign (usually rodent) constant regions have been replaced with sequences of human origin, have been shown to be less immunogenic than antibodies of fully foreign origin, and the most development efforts in therapeutic antibodies are trending towards the use of humanized or fully human antibodies. Preferably, chimeric antibodies or other antibodies of non-human origin are humanized to reduce the risk of a human anti-antibody response. For chimeric antibodies, humanization may include, for example, modification of the framework regions of the variable region sequences. Amino acid residues of a CDR may often not be altered during humanization, although in certain cases it may be desirable to alter individual CDR amino acid residues, for example to remove a glycosylation site, a deamidation site or an undesired cysteine residue.

Numerous methods for humanization of an antibody sequence are known in the art. A commonly used method is CDR grafting, which may involves identification of human germline gene counterparts to murine variable region genes and grafting of the murine CDR sequences into this framework. Since CDR grafting reduces the chance for binding specificity and affinity and the biological activity of a CDR grafted non-human antibody, back mutations are often introduced at selected positions of the CDR grafted antibody to retain the binding specificity and affinity. Amino acid residues that for back mutations may include those that are located at the surface of an antibody molecule. Another humanization technique for CDR grafting and back mutation is resurfacing, in which non-surface exposed residues of non-human origin are retained, while surface residues are altered to human variants.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat [symbol] Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL(L1, L2, L3). Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject or individual is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (K) and lambda (A), based on the amino acid sequence of its constant domain.

The term “package insert” is used to refer to instructions customarily included in commercial packages of therapeutic products, that contain information about the indications, usage, dosage, administration, combination therapy, contraindications, and/or warnings concerning the use of such therapeutic products.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, “administering” is meant a method of giving a dosage of a compound (e.g., an anti-CD3 antibody of the invention or a nucleic acid encoding an anti-CD3 antibody of the invention) or a composition (e.g., a pharmaceutical composition, e.g., a pharmaceutical composition including an anti-CD3 antibody of the invention) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

As used herein, “somatic hypermutation” or “SHM” refers to the mutation of a polynucleotide sequence initiated by, or associated with the action of the Activation-Induced Cytidine Deanimase (AID), a functional AID mutant, uracil glycosylase and/or error prone polymerases on that polynucleotide sequence. As used herein, the term includes mutagenesis that occurs as a consequence of the error prone repair, including mutagenesis mediated by the mismatch repair machinery and related enzymes.

SHM is generally initiated by targeting AID to rearranged V(D)J and switch regions of Ig genes. The mutation rate of this programmed mutagenesis is a million-fold higher than the non-AID targeted genome of B cells. AID is a processive enzyme that binds single-stranded DNA and deaminates cytosines in DNA. Cytosine deamination generates highly mutagenic deoxy-uracil (U) in the DNA of the Ig loci. Mutagenic processing of uracil through the DNA damage response produces the entire spectrum of base substitutions, which characterizes SHM at and around an initial U lesion. At least five, identified mutagenic DNA damage response pathways are known to generate a well-defined SHM spectrum of C/G transitions, C/G transversions, and A/T mutations around this initial lesion. These pathways include (1) replication opposite template U generates transitions at C/G, (2) UNG2-dependent translesion synthesis (TLS) generates transversions at C/G, (3) a hybrid pathway comprising non-canonical mismatch repair (ncMMR) and UNG2-dependent TLS generates transversions at C/G, (4) ncMMR generates mutations at A/T, and (5) UNG2- and PCNA Ubiquitination (PCNA-Ub)-dependent mutations at A/T. Specific strand-biases of SHM spectra arise as a consequence of a biased AID targeting, ncMMR, and anti-mutagenic repriming. By elucidating the amino acid and/or nucleotide sequences of the CDR3 variable regions and/or one of CDR3 heavy chain (HC) and light chain (LC or λ) variable regions of the anti-CD3 antibodies disclosed herein, the present inventors identified a series of “clusters” or “motifs” within the sequences. These clusters represent convergent somatic hypermutations (SHM) in the variable region sequences. Clustering may provide insight into the functionally related sequences and the diversity of the total population of antibodies and their variable region sequences. Sequences that are descendants from the same parent B cell or convergently evolved the sequences in the same cluster should be functionally more related than sequences belonging to other clusters. Convergent SHMs are likely functionally related mutations, e.g., they share a specific affinity for CD3. These SHM may inform the development of recombinant anti-CD3 antibodies with improved properties, such as specific binding for CD3 fragments.

### Selected Embodiments

One aspect of the invention relates to various novel anti-CD3 antibodies and fragments thereof.

The presently disclosed antibodies may provide treatments that are far more effective than current therapies. The presently disclosed CD3 antibodies of the invention may be included as part of a treatment regime, which may include, for example, providing two or more such antibodies, and/or in combination with other treatments such as chemotherapy.

In one aspect, the invention relates to novel CD3 antigen binding peptides, which may be antibodies and/or fragments thereof. In certain aspects, the antibodies and/or fragments thereof bind to a CD3 fragment having an amino acid sequence of SEQ ID NO: 1, and/or a sequence having at least 85%, at least 90% at least 95%; at least 96%, at least 97%, at least 98% or at least 99% sequence identity to SEQ ID NO: 1. In certain aspects, the antibodies and/or fragments thereof bind to a CD3 fragment having an amino acid sequence of SEQ ID NO: 234, and/or a sequence having at least 85%, at least 90% at least 95%; at least 96%, at least 97%, at least 98% or at least 99% sequence identity to SEQ ID NO: 234. In certain aspects, the antibodies and/or fragments thereof bind to a CD3 fragment having an amino acid sequence of SEQ ID NO: 235, and/or a sequence having at least 85%, at least 90% at least 95%; at least 96%, at least 97%, at least 98% or at least 99% sequence identity to SEQ ID NO: 235.

In certain aspects, the present invention provides an anti-CD3 antibody or antibody fragment comprising a heavy chain CDR3 sequence (VH) comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59. Additionally or alternatively, an anti-CD3 antibody or antibody fragment of the present invention may comprise a light chain CDR3 sequence (VL or 2) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

Additionally or alternatively, the present invention provides an anti-CD3 antibody or antibody fragment comprising a heavy chain CDR3 sequence (VH) comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59 and a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

Additionally or alternatively, an anti-CD3 antibody or antibody fragment of the present invention may comprise a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175. Additionally or alternatively, an anti-CD3 antibody or antibody fragment of the present invention may comprise a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

Additionally or alternatively, an anti-CD3 antibody or antibody fragment of the present invention may comprise a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175 and a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

Additionally or alternatively, an anti-CD3 antibody or antibody of the present comprises a heavy chain variable region and/or a light chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.1 and/or Clust 8.2, as set forth in Tables 3-4.

In certain aspects, the present invention provides compositions, including therapeutic compositions, comprising an anti-CD3 antibody or antibody fragment as described herein. In certain aspects, the present invention provides compositions, including therapeutic compositions, two or more of the CD3 antibodies disclosed herein. Certain compositions of the invention include a plurality of different CD3 antibodies, as disclosed herein, wherein each different antibody binds to a distinct CD3 epitope or fragment.

In certain aspects, the present invention provides methods for treating breast cancer using compositions comprising one or more CD3 antibodies, as described herein. In certain aspects, administration of such a composition results in reduced CD3 and/or HER2 expression, CD3 and/or HER2 receptor internalization, and/or ligand-induced phosphorylation of HER3.

In certain aspects, the present invention provides immunoconjugates and/or compositions comprising such immunoconjugates, wherein said immunoconjugates comprise a CD3 antibody of the invention conjugated to another therapeutic agent, such as an anti-cancer agent. The invention further provides immunoconjugates comprising two or more different CD3 antibodies or fragment thereof, wherein each different CD3 antibody or fragment targets a different CD3 fragment or epitope.

A further aspect of the invention relates to a nucleic acid molecule having a nucleotide sequence that encodes a CD3 antibody or fragment thereof, as disclosed herein, as well as expression vectors comprising such a polynucleotide and host cells that have been transfected with such an expression vector.

Aspects of the invention also provide methods for producing the CD3 antibodies, fragments thereof, and compositions of the invention.

The present invention also provides methods for treating a disease in a human or animal subject, in particular treatment of cancer in humans, by administering an anti-CD3 antibody or composition of the invention to said subject. The invention also includes the use of one or more anti-CD3 antibodies of the invention for preparation of a medicament for use in treating a disease in a human or animal, in particular for the treatment of cancer in humans.

Another embodiment of this aspect of the invention relates to an antibody composition comprising at least first and second anti-CD3 antibodies, wherein the first and second antibodies bind distinct epitopes of CD3, said first and second antibodies independently comprising a heavy chain CDR3 sequence (VH) comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59. Additionally or alternatively, said first and second anti-CD3 antibodies comprise a light chain CDR3 sequence (VL or 2) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

Another aspect of the invention relates to nucleic acid molecules comprising a nucleotide sequence that encodes an antibody, VL variable region sequence, VH variable region sequence, VL CDR3 sequence, and/or VH CDR3 sequence as set forth herein, and/or a sequence having an amino acid sequence comprising a sequence having at least 85%, at least 90% at least 95%; at least 96%, at least 97%, at least 98% or at least 99% with any of SEQ ID NOS: 2-233.

A further aspect of the invention relates to an expression vector comprising a nucleic acid molecule as defined above. As noted above, expression vectors for use in the context of the present invention may be of any suitable type known in the art, e.g., a plasmid or a viral vector.

A still further aspect of the invention relates to a host cell comprising a nucleic acid molecule as defined above, wherein said host cell is capable of expressing an anti-CD3 antibody encoded by said nucleic acid molecule.

In some embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁶M or less, e.g., from 10⁻⁸M to 10⁻¹³M, e.g., from 10⁻⁹ M to 10⁻¹³M). In preferred aspects, this affinity range is the “optimal affinity range”, which retains anti-tumor activity but has reduced toxicity due to reduced cytokine release. In certain preferred aspects, the anti-CD3 antibody has an affinity in the range of 30 nM to 40 nM. In preferred aspects, the anti-CD3 antibody has an affinity in the range of 30-40 nM as measured by alanine scanning of the HC CDR3 of the antibody.

In some embodiments, Kd is measured by a radiolabeled antigen binding assay (RIA). In some embodiments, an RIA is performed with the Fab version of an antibody of interest and its antigen. For example, solution binding affinity of Fabs for antigen is measured by equilibrating Fab with a minimal concentration of (1251)-labeled antigen in the presence of a titration series of unlabeled antigen, then capturing bound antigen with an anti-Fab antibody-coated plate (see, e.g., Chen et al., J. Mol. Biol. 293:865-881(1999)).

In some embodiments, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at 10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier [symbol] instructions. Antigen is diluted with 10 mM sodium acetate, pH 4.8, to 5 g/ml (0.2 M) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kon) and dissociation rates (KO are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio kon/koff. See, for example, Chen et al., J. Mol. Biol. 293:865-881 (1999). If the on-rate exceeds 106 M-1s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In preferred aspects, the modality to estimate affinity is production of a monovalent anti-CD3 antibody followed by titration on live CD3 expressing cells and determination of MFI by flow cytometry to determine an EC50 value. Advantageously, this may represent an exacting context (monovalent and CD3 on cells) in which a therapeutic based on the anti-CD3 antibodies of the invention is used.

In some embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, e.g., EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage).

In some embodiments, an antibody provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In preferred embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

The human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animals chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. LISA. 103; 3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

For example, in preferred aspects, technologies such as AlivaMab Mouse technology are used to produce the anti-CD3 antibodies described herein. AlivaMab Mouse technology is used to generate panels of monoclonal antibodies (mAbs) against CD3.

The AlivaMab Mouse is a transgenic mouse that produces chimeric human-mouse monoclonal antibodies comprising fully human Fab and upper hinge regions and mouse middle hinge and Fc regions. Optimized constant domains facilitate the generation and identification of antibodies that retain structure-function characteristics. Antibodies of the invention produced using AlivaMab Mouse technology possess biophysical properties, which are predictive and comparable to that of fully human antibody counterparts.

Antibodies of the invention may be produced by AlivaMab Kappa Mice, and may include a chimeric immunoglobulin heavy (IgH) chain and a human immunoglobulin kappa (IgK) light chain. The antibodies of the invention produced by AlivaMab Lambda Mice may include a chimeric IgH chain and a human immunoglobulin lambda (IgK) light chain. The chimeric IgH chain of the AlivaMab Mouse anti-CD3 antibodies may include a human variable region comprising a human variable heavy (VH) domain, a human diversity heavy (DH) domain, and a human joining heavy (JH) domain, a human constant heavy 1 (CH1) domain, a human upper hinge region (except for Oμ, which is naturally missing an upper hinge region), a mouse middle hinge region, a mouse CH2 domain, and a mouse CH3 domain.

When an anti-CD3 antibody is discovered, the human heavy chain variable region is readily appended to a fully human constant region while maintaining the antigen-binding characteristics of the parent chimeric antibody that were developed in vivo in the AlivaMab Mouse. In one embodiment, the human heavy chain variable region, CH1 and, optionally, upper hinge region of the chimeric antibody are appended to human hinge, a human CH2 domain and a human CH3 domain in order to produce a fully human anti-CD3 antibody as disclosed herein.

Portions of variable regions from the antibodies produced from AlivaMab Mouse technology may include all or a combination of the complementarity determining regions (CDRs) of the VH and/or VL. The variable regions may be formatted with constant regions, either native or modified for various desired effector functions, in a standard antibody structure (two heavy chains with two light chains). The variable regions may also be formatted as multi-specific antibodies, e.g., bispecific antibodies binding to two different epitopes or to two different antigens. The variable regions may also be formatted as antibody fragments, e.g., single-domain antibodies comprising a single VH or VL, Fabs or Fab′2. The antibodies may also be used as antibody-drug conjugates, or carry other additions such as small molecule toxins, biologic toxins, cytokines, oligopeptides, or RNAs to increase therapeutic modality and/or increase safety.

Methods for producing the anti-CDR3 antibodies of the invention using AlivaMab mouse technology may include immunizing AlivaMab Kappa Mice and AlivaMab Lambda Mice with an antigen. Generally, within two weeks, the mice are sacrificed and terminal materials collected. Spleens and lymph nodes may be prepared and fused with myeloma cells (such as CRL-2016 cells) using a PEG based method as generally described in “Antibodies: A Laboratory Manual” (Harlow and Lane 1988 CSH Press) to establish hybridomas.

Hybridomas may be grown in 384-well tissue culture plates and supernatants from individual wells were screened by ELISA for production of antibodies recognizing the antigen of interest. Positive wells are then transferred to 48-well plates, expanded, and supernatants were collected for antigen binding confirmation by ELISA. Positive supernatants may also be counter-screened against a non-related histidine-tagged protein. Hybridoma lines each from AlivaMab Kappa Mice and AlivaMab Lambda Mice are confirmed to bind to the antigen specifically by ELISA and are picked at random and single-cell cloned into 96-well plates. They are grown into colonies and the supernatant from these individual colonies is screened by ELISA to re-confirm monoclonal antibody binding to the antigen of interest. These supernatants are then screened by FACS to confirm binding to the native antigen expressed on cells.

AlivaMab Mouse technology and methods for producing antibodies using such technologies can be found in WO 2010/039900 and WO 2011/123708, which are incorporated herein in their entirety.

In certain methods for designing and/or producing the anti-CD3 antibodies of the invention, by elucidating the amino acid and/or nucleotide sequences of the CDR3 variable regions and/or one of CDR3 heavy chain (HC) and light chain (LC or 2) variable regions of the anti-CD3 antibodies produced, for example, using AlivaMab Mouse technology, a series of “clusters” or “motifs” are identified within the sequences. These clusters represent convergent somatic hypermutations (SHM) in the variable region sequences. Clustering may provide insight into the functionally related sequences and the diversity of the total population of antibodies and their variable region sequences. Sequences that are descendants from the same parent B cell or convergently evolved the sequences in the same cluster should be functionally more related than sequences belonging to other clusters. Convergent SHMs are likely functionally related mutations, e.g., they share a specific affinity for CD3. These SHM may inform the development of recombinant anti-CD3 antibodies with improved properties, such as specific binding for CD3 fragments.

Specific SHMs in the anti-CD3 antibody variable regions of the antibodies disclosed herein are found in Tables 1-4, with the amino acids representing an SHM indicated in bold.

Human antibodies may also be generated by isolating Fv clone variable domain sequences selected from human-derived phage display libraries. Such variable domain sequences may then be combined with a desired human constant domain.

Antibodies of the invention may be isolated by screening combinatorial libraries for antibodies with the desired activity or activities. For example, a variety of methods is known in the art for generating phage display libraries and screening such libraries for antibodies possessing the desired binding characteristics. Such methods are reviewed, e.g., in Hoogenboom et al. in Methods in Molecular Biology 178:1-37 (O [symbol] rien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Alternatively, in certain aspects, the present invention includes humanized variants of the antibodies described herein or humanized antibodies comprising a one or more of SEQ ID NOS: 2-233, or a fragment thereof. Methods for humanizing antibodies are well known in the art.

In certain aspects, the anti-CD3 antibodies of the invention are, or form a part of, a multispecific antibody.

Multispecific antibodies are monoclonal antibodies that have binding specificities for at least two different sites. In some embodiments, bispecific antibodies may bind to two different epitopes of CD3 (e.g., CD3δ or CD3γ). In some embodiments, one of the binding specificities is for CD3 (e.g., CD3δ or CD3γ) and the other is for any other antigen (e.g., a second biological molecule, e.g., a cell surface antigen, e.g., a tumor antigen). Accordingly, a bispecific anti-CD3 antibody may have binding specificities for CD3 and a second biological molecule, such as a second biological molecule (e.g., a tumor antigen).

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies of the invention may be an anti-CD3 arm in one embodiment. Alternatively, the knob of the multispecific antibodies of the invention may be an anti-target/antigen arm in one embodiment. The hole of the multispecific antibodies of the invention may be an anti-CD3 arm in one embodiment. Alternatively, the hole of the multispecific antibodies of the invention may be an anti-target/antigen arm in one embodiment.

There other ways of making multispecific antibodies. For example, multispecific antibodies may be engineered using immunoglobulin crossover (also known as Fab domain exchange or CrossMab format) technology (see e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. ImmunoL, 148(5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. ImmunoL, 152:5368 (1994)); and preparing trispecific antibodies as described, e.g., in Tutt et al. J. ImmunoL 147: 60 (1991).

In certain embodiments, amino acid sequence variants of the anti-CD3 antibodies of the invention (e.g., bispecific anti-CD3 antibodies of the invention that bind to CD3 and a second biological molecule, e.g., a cell surface antigen, e.g., a tumor antigen, such as certain members and/or fragments of the epidermal growth factor receptor (HER/EGFR/ERBB) family, and/or different CD3 epitopes) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain aspects, the anti-CD3 antibodies of the invention are multi-specific antibodies. In certain aspects, the multi-specific antibodies are bispecific antibodies, trispecific antibodies, and/or of greater multi-specificity that exhibit specificity to CD3 and another molecule and/or another epitope of CD3. For example, such antibodies can bind to both CD3 and to an antigen that is important for targeting the antibody to a particular cell type or tissue (for example, to an antigen associated with a cancer antigen of a tumor being treated). In some embodiments, multi-specific antibodies of the invention bind to molecules (receptors or ligands) involved in immunomodulatory pathways, such as CTLA4, TIM3, TIM4, OX40, CD40, GITR, 4-1-BB, CD27/CD70, ICOS, B7-H4, LIGHT, PD-1 or LAG3, which may provide control or modulation of the multi-specific antibodies' therapeutic effects. Furthermore, a multispecific antibody may bind to effecter molecules such as cytokines (e.g., IL-7, IL-15, IL-12, IL-4 TGF-beta, IL-10, IL-17, IFNg, Flt3, BLys) and/or chemokines (e.g., CCL21). Methods are known in the art for producing bispecific antibodies.

In certain embodiments, antibody variants having one or more amino acid substitutions are provided. Sites of interest for substitutional mutagenesis include the HVRs and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE A
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen may be used. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In certain embodiments, anti-CD3 antibodies of the invention can be altered to increase or decrease the extent to which the antibody is glycosylated. Addition or deletion of glycosylation sites to anti-CD3 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, anti-CD3 antibody variants are provided having a carbohydrate structure that lacks fucose attached (directly or indirectly) to an Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about 3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lecl3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-CD3 antibodies variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an anti-CD3 antibody of the invention thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions. In certain embodiments, the invention contemplates an anti-CD3 antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat [symbol] Acad. Sci. USA 83:7059-7063 (1986)) and Hellstrom, I et al., Proc. Nat [symbol] Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96 non-radioactive cytotoxicity assay (Promega, Madison, Wis.). Useful effector cells for such assays include peripheral blood mononuclear cells (PBMC) and Natural Killer (NK) cells.

Alternatively, or additionally, ADCC activity of the molecule of interest may be assessed in vivo, e.g., in an animal model such as that disclosed in Clynes et al. Proc. Nat [symbol] Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. ImmunoL Methods 202:163 (1996); Cragg, M. S. et al. Blood. 101: 1045-1052 (2003); and Cragg, M. S. and M. J. Glennie Blood. 103:2738-2743 (2004)). FcRn binding and in vivo clearance/half life determinations can also be performed using methods known in the art (see, e.g., Petkova, S. B. et al. Int1. ImmunoL 18(12): 1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al.: Nature 406, 267-273 (20 Jul. 2000)). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some aspects the bispecific antibody comprises an Fc region comprising an N297G mutation. In some embodiments, the bispecific antibody comprising the N297G mutation comprises one or more heavy chain constant domains, wherein the one or more heavy chain constant domains are selected from a first CH1 domain, a first CH2 domain, a first CH3 domain, a second CH1 domain, second CH2 domain, and a second CH3 domain.

In certain embodiments, it may be desirable to create cysteine engineered antibodies in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in WO 2016/040856, which is incorporated by reference in its entirety herein, including any drawings.

In certain embodiments, the bispecific antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

The bispecific antibodies of the invention may be produced using recombinant methods and compositions, for example, as described in U.S. Pat. No. 4,816,567. In one embodiment, an isolated nucleic acid encoding an anti-CD3 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20 cell). In one embodiment, a method of making a bispecific antibody is provided, wherein the method comprises culturing a host cell comprising a nucleic acid encoding the antibody under conditions suitable for expression of the antibody, and optionally recovering the antibody from the host cell (or host cell culture medium).

For recombinant production of an antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

The antibodies of the invention may be identified, screened for, or characterized for their physical/chemical properties and/or biological activities by various assays known in the art.

In one aspect, the antibody of the invention is tested for its antigen binding activity, for example, by known methods such as ELISA, Western blot, etc. In another aspect, competition assays may be used to identify an antibody that competes with an anti-CD3 antibody of the invention for binding to CD3. In an exemplary competition assay, immobilized CD3 is incubated in a solution comprising a first labeled antibody that binds to CD3 and a second unlabeled antibody that is being tested for its ability to compete with the first antibody for binding to CD3. The second antibody may be present in a hybridoma supernatant. As a control, immobilized CD3 is incubated in a solution comprising the first labeled antibody but not the second unlabeled antibody. After incubation under conditions permissive for binding of the first antibody to CD3, excess unbound antibody is removed, and the amount of label associated with immobilized CD3 is measured. If the amount of label associated with immobilized CD3 is substantially reduced in the test sample relative to the control sample, then that indicates that the second antibody is competing with the first antibody for binding to CD3. See, e.g., Harlow and Lane (1988) Antibodies: A Laboratory Manual. Ch. 14 (Cold Spring Harbor Laboratory, Cold Spring Harbor, N.Y.).

In one aspect, assays are provided for identifying antibodies having biological activity. Biological activity may include, for example, binding to CD3 (e.g., CD3 on the surface of a T cell), or a peptide fragment thereof, either in vivo, in vitro, or ex vivo. In the case of a bispecific antibody of the invention, biological activity may also include, for example, effector cell activation (e.g., T cell (e.g., CD8+ and/or CD4+ T cell) activation), effector cell population expansion (i.e., an increase in T cell count), target cell population reduction (i.e., a decrease in the population of cells expressing the second biological molecule on their cell surfaces), and/or target cell killing. In some embodiments, the activity comprises ability to support B cell killing and/or the activation of the cytotoxic T cells.

In certain embodiments, any of the antibodies of the invention may be used to detect the presence of CD3 in a biological sample. The term “detecting” as used herein encompasses quantitative or qualitative detection. In certain embodiments, a biological sample comprises a cell or tissue. In certain embodiments, the method comprises contacting the biological sample with an anti-CD3 antibody as described herein under conditions permissive for binding of the bispecific antibody to CD3 and another antigen, and detecting whether a complex is formed between the bispecific antibody and CD3. Such method may be an in vitro or in vivo method.

In certain embodiments, labeled antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase, and bacterial luciferase (see for example, U.S. Pat. No. 4,737,456, which is incorporated by reference in its entirety herein, including any drawings), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Production of Anti-CD3 Antibodies and Antibody Compositions

An additional aspect of the invention relates to methods for producing an anti-CD3 antibodies and compositions of the invention. One embodiment of this aspect of the invention relates to a method for producing an anti-CD3 antibody as defined herein, comprising providing a host cell capable of expressing an anti-CD3 antibody, cultivating said host cell under conditions suitable for expression of the antibody, and isolating the resulting antibody.

An antibody or antibody composition of the present invention may be produced by methods generally known in the art for production of recombinant monoclonal or polyclonal antibodies. Thus, in the case of production of a single antibody of the invention, any method known in the art for production of recombinant monoclonal antibodies may be used. For production of an antibody composition comprising two or more anti-CD3 antibodies of the invention, the individual antibodies may be produced separately, i.e., each antibody being produced in a separate bioreactor, or the individual antibodies may be produced together in single bioreactor. When the number of different antibodies in a composition is more than e.g., two or three, it will generally be preferably for reasons of cost efficiency to produce the antibodies together in a single bioreactor. On the other hand, when the composition only contains a small number of different antibodies, e.g., two, three or possibly four different antibodies, a decision to produce them separately in different bioreactors or together in a single bioreactor will have to be made based on the individual circumstances. If the antibody composition is produced in more than one bioreactor, the purified anti-CD3 antibody composition can be obtained by pooling the antibodies obtained from individually purified supernatants from each bioreactor. Various approaches are known in the art for production of a polyclonal antibody composition in multiple bioreactors, where the cell lines or antibody preparations are combined at a later point upstream or prior to or during downstream processing.

In the case of production of two or more individual antibodies in a single bioreactor, this may be performed, for example, based on site-specific integration of the antibody coding sequence into the genome of the individual host cells, ensuring that the V_{H} and V_{L} protein chains are maintained in their original pairing during production. Furthermore, the site-specific integration minimizes position effects, and therefore the growth and expression properties of the individual cells in the polyclonal cell line are expected to be very similar. Generally, the method involves the following: i) a host cell with one or more recombinase recognition sites; ii) an expression vector with at least one recombinase recognition site compatible with that of the host cell; iii) generation of a collection of expression vectors by transferring the selected V_{H} and V_{L} coding pairs from the screening vector to an expression vector such that a full-length antibody or antibody fragment can be expressed from the vector (such a transfer may not be necessary if the screening vector is identical to the expression vector); iv) transfection of the host cell with the collection of expression vectors and a vector coding for a recombinase capable of combining the recombinase recognition sites in the genome of the host cell with that in the vector; v) obtaining/generating a polyclonal cell line from the transfected host cell and vi) expressing and collecting the antibody composition from the polyclonal cell line.

An alternative approach is to produce two or more different antibodies in a single bioreactor. This method involves generation of a polyclonal cell line capable of expressing a polyclonal antibody or other polyclonal protein comprising two or more distinct members by a) providing a set of expression vectors, wherein each of said vectors comprises at least one copy of a distinct nucleic acid encoding a distinct member of the polyclonal protein, separately transfecting host cells with each of the expression vectors under conditions avoiding site-specific integration of the expression vectors into the genome of the cells, thereby obtaining two or more compositions of cells, each composition expressing one distinct member of the polyclonal protein, and c) mixing the at least two compositions of cells to obtain a polyclonal cell line.

The antibodies of the invention may be produced in various types of cells, including mammalian cells as well as non-mammalian eukaryotic or prokaryotic cells, such as plant cells, insect cells, yeast cells, fungi, E. coli etc. However, the antibodies are preferably produced in mammalian cells, for example CHO cells, COS cells, BHK cells, myeloma cells (e.g., Sp2/0 or NSO cells), fibroblasts such as NIH 3T3, or immortalized human cells such as HeLa cells or HEK 293 cells.

Methods for transfecting a nucleic acid sequence into a host cell are well-known in the art (see, e.g., Sambrook et al., Molecular Cloning: A Laboratory Manual, Cold Spring Harbor Laboratory Press, 3rd Edition, 2001). For site-specific integration a suitable host cell will comprise one or more recombinase recognition sites in its genome. In this case, a suitable expression vector comprises a recombination recognition site matching the recombinase recognition site(s) of the host cell.

One embodiment of the present invention is thus a polyclonal cell line capable of expressing two or more anti-CD3 antibodies of the present invention. A further embodiment is a polyclonal cell line wherein each individual cell is capable of expressing a single V_{H} and V_{L} pair, and the polyclonal cell line as a whole is capable of expressing a collection of V_{H} and V_{L} pairs, where each V_{H} and V_{L} pair encodes an anti-CD3 antibody.

### Therapeutic Compositions

Another aspect of the invention is a pharmaceutical composition comprising as an active ingredient at least one anti-CD3 antibody of the invention, or an anti-CD3 Fab or another anti-CD3 antibody fragment composition. Such compositions are intended for amelioration, prevention and/or treatment of cancer. The pharmaceutical composition may be administered to a human or to a domestic animal.

In addition to at least one antibody of the invention or fragment thereof, the pharmaceutical composition will further comprise at least one pharmaceutically acceptable diluent, carrier or excipient. These may for example include preservatives, stabilizers, surfactants/wetting agents, emulsifying agents, solubilizers, salts for regulating the osmotic pressure and/or buffers. Solutions or suspensions may further comprise viscosity-increasing substances, such as sodium carboxymethylcellulose, carboxymethylcellulose, dextran, polyvinylpyrrolidone or gelatin. A suitable pH value for the pharmaceutical composition will generally be in the range of about 5.5 to 8.5, such as about 6 to, 8, e.g., about 7, maintained where appropriate by use of a buffer.

Conventional pharmaceutical practice may be employed to provide suitable formulations or compositions to administer to e.g., cancer patients. The administration will typically be therapeutic, meaning that it is administered after a cancer condition has been diagnosed. Any appropriate route of administration may be employed, for example parenteral, intravenous, intra-arterial, subcutaneous, intramuscular, intraperitoneal, intranasal, aerosol, suppository or oral administration. Pharmaceutical compositions of the invention will typically be administered in the form of liquid solutions or suspensions, more typically aqueous solutions or suspensions, in particular isotonic aqueous solutions or suspensions.

As an alternative to a liquid formulation, the compositions of the invention may be prepared in lyophilized form comprising the at least one antibody alone or together with a carrier, for example mannitol, in which case the composition is reconstituted with a liquid such as sterile water prior to use.

The pharmaceutical compositions comprise from approximately 1% to approximately 95%, preferably from approximately 20% to approximately 90%, active ingredient. Pharmaceutical compositions according to the invention may e.g., be produced in unit dose form, such as in the form of ampoules, vials, suppositories, tablets or capsules. The formulations can be administered to human individuals in therapeutically or prophylactically effective amounts (e.g., amounts which prevent, eliminate, or reduce a pathological condition) to provide therapy for a cancerous disease or other condition. The preferred dosage of therapeutic agent to be administered is likely to depend on such variables as the severity of the cancer, the overall health status of the particular patient, the formulation of the compound excipients, and its route of administration.

### Therapeutic Uses of Antibodies and Compositions According to the Invention

The anti-CD3 antibodies and pharmaceutical compositions according to the present invention may be used for the treatment or amelioration of a disease, in a mammal, in particular treatment of cancer in humans.

Some embodiments provide a method of treating or delaying the progression of a cell proliferative disorder or an autoimmune disorder in a subject in need thereof, the method comprising administering to the subject an effective amount any one of the antibodies described herein (in a monospecific, bi-specific, or multi-specific format). In another aspect, the invention features a method of enhancing or decreasing immune function in a subject having a cell proliferative disorder or an autoimmune disorder, the method comprising administering to the subject any one of the antibodies described herein (in a monospecific, bi-specific, or multi-specific format).

In any of the uses or methods set forth herein, the cell proliferative disorder can be cancer. In some embodiments, the cancer is selected from the group consisting of breast cancer, colorectal cancer, non-small cell lung cancer, non-Hodgkin [symbol] lymphoma (NHL), B cell lymphoma, B cell leukemia, multiple myeloma, renal cancer, prostate cancer, liver cancer, head and neck cancer, melanoma, ovarian cancer, mesothelioma, glioblastoma, germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In some embodiments, the preferred cancer is germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt [symbol] lymphoma (BL).

In some embodiments, the autoimmune disorder is selected from the group consisting of rheumatoid arthritis, juvenile rheumatoid arthritis, systemic lupus erythematosus (SLE), Wegeners disease, inflammatory bowel disease, idiopathic thrombocytopenia purpura (ITP), thrombotic thrombocytopenia purpura (TTP), autoimmune thrombocytopenia, multiple sclerosis, psoriasis, IgA nephropathy, IgM polyneuropathies, myasthenia gravis, vasculitis, diabetes mellitus, Reynaud [symbol] syndrome, Sjorgen [symbol] syndrome, glomerulonephritis, Neuromyelitis Optica (NMO), and IgG neuropathy.

In some embodiments, the antibody is in a kit comprising: (a) a composition comprising any one of the antibodies described herein (in a monospecific, bi-specific, or multi-specific format) and (b) a package insert comprising instructions for administering the composition to a subject to treat or delay progression of a cell proliferative disorder. In some embodiments, the antibody within the kit is lyophilized.

In any of the preceding uses or methods, the subject can be a human.

### Dose and Route of Administration

The antibodies and compositions of the invention will be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the anti-CD3 antibodies are being administered as a stand-alone treatment or in combination with one or more additional anti-cancer treatments.

An effective amount for tumor therapy may be measured by its ability to stabilize disease progression and/or ameliorate symptoms in a patient, and preferably to reverse disease progression, e.g., by reducing tumor size. The ability of an antibody or composition of the invention to inhibit cancer may be evaluated by in vitro assays, e.g., as described in the examples, as well as in suitable animal models that are predictive of the efficacy in human tumors. Suitable dosage regimens will be selected in order to provide an optimum therapeutic response in each particular situation, for example, administered as a single bolus or as a continuous infusion, and with possible adjustment of the dosage as indicated by the exigencies of each case.

In some embodiments, the antibody is administered to the subject in a dosage of about 0.01 mg/kg to about 10 mg/kg. In some embodiments, the antibody is administered to the subject in a dosage of about 0.1 mg/kg to about 10 mg/kg. In some embodiments, the antibody is administered to the subject in a dosage of about 1 mg/kg. In some embodiments, the antibody is administered subcutaneously, intravenously, intramuscularly, topically, orally, transdermally, intraperitoneally, intraorbitally, by implantation, by inhalation, intrathecally, intraventricularly, or intranasally. In some embodiments, the antibody is administered subcutaneously. In some embodiments, the bispecific antibody is administered intravenously.

### Pharmaceutical Formulations

Pharmaceutical formulations of the antibodies of the invention may be prepared by mixing such antibody having the desired degree of purity with one or more optional pharmaceutically acceptable carriers (Remington [symbol] Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended. Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington s Pharmaceutical Sciences 16th edition, Osol, A. Ed. (1980).

Sustained-release preparations may be prepared. Suitable examples of sustained-release preparations include semipermeable matrices of solid hydrophobic polymers containing the bispecific antibody, which matrices are in the form of shaped articles, for example, films, or microcapsules. The formulations to be used for in vivo administration are generally sterile. Sterility may be readily accomplished, e.g., by filtration through sterile filtration membranes.

### EXAMPLES

### Example 1

The present example provides the results of a hybridoma-based method used to generate multiple, different anti-CD3 antibodies of the invention.

Briefly, the multiple, different anti-CD3 antibodies were produced using hybridoma technology. Although the present inventors have used this methodology to produce a large number of anti-CD3 antibodies, this Example provides 58 CD3-specific antibodies discovered by the present inventors. By elucidating the amino acid and/or nucleotide sequences of these antibodies' CDR3 heavy chain (HC) and light chain (LC or 2) variable regions, the present inventors identified a series of “clusters” or “motifs” within the sequences. These clusters represent convergent somatic hypermutations (SHM) in the variable region sequences. Clustering may provide insight into the functionally related sequences and the diversity of the total population of antibodies and their variable region sequences. Sequences that are descendants from the same parent B cell or convergently evolved the sequences in the same cluster should be functionally more related than sequences belonging to other clusters.

In the present Example, the variable chain region sequences were provided by different antibodies. Thus, any clustering can most likely be attributed to convergent SHM, which are likely functionally related mutations, e.g., they share a specific affinity for CD3. These SHM may inform the development of recombinant anti-CD3 antibodies with improved properties, such as specific binding for CD3 fragments.

### Hybridomas

Hybridomas were produced that each expressed a different anti-CD3 antibody. Methods known in the art to generate antibodies and/or hybridoma cells are known in the art.

For example, different anti-CD3 antibodies are obtained from different populations of substantially homogeneous antibodies, i.e., the individual antibodies comprising the population are identical and/or bind the same epitope except for possible variants that arise during production of the monoclonal antibody, such variants generally being present in minor amounts.

By way of example, in certain hybridoma methods, a mouse or other appropriate host animal, such as a hamster, is immunized with a CD3 antigen to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from mouse tumors. Human myeloma and mouse-human heteromyeloma cell lines also have been described for the production of human monoclonal antibodies.

Culture medium in which hybridoma cells are growing is assayed for production of monoclonal antibodies directed against CD3. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells.

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences, or by covalently joining to the immunoglobulin coding sequence all or part of the coding sequence for a non-immunoglobulin polypeptide.

### Sequencing

Briefly, a cDNA library was created from anti-CD3 antibody mRNA obtained from a hybridoma culture of cells that express different anti-CD3 antibodies.

The sequences were aligned and all the unique anti-CD3 antibody sequences identified. Alignments the sequences revealed the uniqueness of each particular sequence and their corresponding antibodies. The antibody variable sequences of each different antibody were analyzed using a custom AlivaAlign sequencing software.

The identified CDR3 HC and VL sequences are provided in Table 1 as SEQ ID NOS: 2-59 and 60-117. The corresponding heavy chain variable region and light chain variable region sequences are provided in Table 2 as SEQ ID NOS: 118-175 and 176-233 respectively. As shown in Table 2, the heavy and light chain variable sequences were paired and identified to a particular antibody.

### Clustering

The HC and VL chain variable region sequences were clustered using an algorithm that assigned sequences to a cluster when they shared identical hV, hJ, IV, and IJ genes, had identical HCDR3 lengths and were at least 90% identical (Hamming distance) for their HCDR3s within the cluster.

Table 3 provides the identified SMH for each heavy and light chain variable region sequence. In Table 3, the respective HC and VL variable region sequences are identifiable and pairable by the identity of their associated CD3 antibody.

Table 4 provides data that informs the listed clusters, i.e., 1-8, of the CD3 antibodies. The table also shows gene usage for heavy and light chains as well as difference to germline sequences as a percentage. The corresponding and paired HC and VL CDR3 sequences are also provided. Within each cluster are related sequences or “subclusters”, e.g., 1.1 and 1.2. Surprisingly, some antibodies provided identical heavy and light chain nucleotide sequences between the antibodies.

TABLE 1
CDR3 Sequences

| CDR3 VH Amino | | |
|---|---|---|
| Acid Sequence | Type | SEQ ID NO: |
| ARMRYNYFDY | VH | SEQ ID NO: 2 |
| ARVKWDLLDY | VH | SEQ ID NO: 3 |
| AREQWEPLYFDY | VH | SEQ ID NO: 4 |
| AHLAHWGPYFDY | VH | SEQ ID NO: 5 |
| ARFSNWGSGGYFDY | VH | SEQ ID NO: 6 |
| ARERWALLFFEY | VH | SEQ ID NO: 7 |
| AREKWELLYFDY | VH | SEQ ID NO: 8 |
| ARERWELLFFDY | VH | SEQ ID NO: 9 |
| AREKWVQLYFDF | VH | SEQ ID NO: 10 |
| ARFSNWGSGGYFDY | VH | SEQ ID NO: 11 |
| ARMRYNYFDY | VH | SEQ ID NO: 12 |
| ARVVRDYGMDV | VH | SEQ ID NO: 13 |
| ARERWELLFFDY | VH | SEQ ID NO: 14 |
| AREKWELLYFDY | VH | SEQ ID NO: 15 |
| AREKWVQLYFDF | VH | SEQ ID NO: 16 |
| AREQWELLYFDY | VH | SEQ ID NO: 17 |
| ARGGVGRNYYYY | VH | SEQ ID NO: 18 |
| GMDV | | |
| ARDGRWELLAFDI | VH | SEQ ID NO: 19 |
| AKMRYNYFDY | VH | SEQ ID NO: 20 |
| AKMRYNYFDY | VH | SEQ ID NO: 21 |
| AREQWELLYFDY | VH | SEQ ID NO: 22 |
| AREQWELLYFDY | VH | SEQ ID NO: 23 |
| ARMRYNYFDY | VH | SEQ ID NO: 24 |
| ARERYNYFDY | VH | SEQ ID NO: 25 |
| ARERYNYFDY | VH | SEQ ID NO: 26 |
| ARERYNYFDY | VH | SEQ ID NO: 27 |
| ARERYNYFDF | VH | SEQ ID NO: 28 |
| AREPYNYFDY | VH | SEQ ID NO: 29 |
| ARERYNYFDY | VH | SEQ ID NO: 30 |
| ARERYNYFDY | VH | SEQ ID NO: 31 |
| ARERYNYFDY | VH | SEQ ID NO: 32 |
| ARERYNYFDS | VH | SEQ ID NO: 33 |
| ARVRRNYGMDV | VH | SEQ ID NO: 34 |
| ARVRRNYGMDV | VH | SEQ ID NO: 35 |
| ARVRRNYGMDV | VH | SEQ ID NO: 36 |
| ARERWELLYFDY | VH | SEQ ID NO: 37 |
| ARVRRNYGMDV | VH | SEQ ID NO: 38 |
| ARVRRNYGMDV | VH | SEQ ID NO: 39 |
| ARVRRNYGMDV | VH | SEQ ID NO: 40 |
| ARVRRNYDMDV | VH | SEQ ID NO: 41 |
| ARVRRNYGMDV | VH | SEQ ID NO: 42 |
| AREQWVLLYFDY | VH | SEQ ID NO: 43 |
| ARVRRNYGMDV | VH | SEQ ID NO: 44 |
| ARVRRNYGMDV | VH | SEQ ID NO: 45 |
| ARVRRNYGMDV | VH | SEQ ID NO: 46 |
| ARERWELLYFDY | VH | SEQ ID NO: 47 |
| ARERKYPLQFDY | VH | SEQ ID NO: 48 |
| VRERKYPLYFDY | VH | SEQ ID NO: 49 |
| ARVRRNYGMDV | VH | SEQ ID NO: 50 |
| ARVRRNYGMDV | VH | SEQ ID NO: 51 |
| ARERKYPLQFDY | VH | SEQ ID NO: 52 |
| ARERKYLLYFDY | VH | SEQ ID NO: 53 |
| ARVRRNYGMDV | VH | SEQ ID NO: 54 |
| ARVRRNYGMDV | VH | SEQ ID NO: 55 |
| ARVRRNYDMDV | VH | SEQ ID NO: 56 |
| ARERKYLLYFDY | VH | SEQ ID NO: 57 |
| SRERWNLLFFDY | VH | SEQ ID NO: 58 |
| ARELWELLYFDY | VH | SEQ ID NO: 59 |
| CDR3 λ chain | | |
| sequences | Type | SEQ ID NO |
| SYDSSNVVF | λ | SEQ ID NO: 60 |
| QAWDSSTHVV | λ | SEQ ID NO: 61 |
| SYDSSNRVF | λ | SEQ ID NO: 62 |
| CSYAGSSTLI | λ | SEQ ID NO: 63 |
| QVWDSSSDHPV | λ | SEQ ID NO: 64 |
| SFDRSNRVF | λ | SEQ ID NO: 65 |
| SYDSSNRVF | λ | SEQ ID NO: 66 |
| SYDSSNRVF | λ | SEQ ID NO: 67 |
| SYDSSNRVF | λ | SEQ ID NO: 68 |
| QVWDSTSDHPV | λ | SEQ ID NO: 69 |
| SYDSSNVVF | λ | SEQ ID NO: 70 |
| SYDSSNHWVF | λ | SEQ ID NO: 71 |
| SYDSSNRVF | λ | SEQ ID NO: 72 |
| SYDSSNRVF | λ | SEQ ID NO: 73 |
| SYDSSNRVF | λ | SEQ ID NO: 74 |
| SYDSSNRVF | λ | SEQ ID NO: 75 |
| GTWDSSLSAVV | λ | SEQ ID NO: 76 |
| QSADTSGTYRV | λ | SEQ ID NO: 77 |
| SYDGSNVVF | λ | SEQ ID NO: 78 |
| SYDGSNVVF | λ | SEQ ID NO: 79 |
| SYDSSNRVF | λ | SEQ ID NO: 80 |
| SYDRSNRVF | λ | SEQ ID NO: 81 |
| SYDSSNVVF | λ | SEQ ID NO: 82 |
| SYDSSNRVF | λ | SEQ ID NO: 83 |
| SYNGNNRVF | λ | SEQ ID NO: 84 |
| SYDNSNRVF | λ | SEQ ID NO: 85 |
| SYDGSNRVF | λ | SEQ ID NO: 86 |
| SYDNSNRVF | λ | SEQ ID NO: 87 |
| SYDSSNRVF | λ | SEQ ID NO: 88 |
| SYDNNNRVF | λ | SEQ ID NO: 89 |
| SYDNSNRVF | λ | SEQ ID NO: 90 |
| SYDGNNRVF | λ | SEQ ID NO: 91 |
| SYDSSNHWVF | λ | SEQ ID NO: 92 |
| SYDSSNHWVF | λ | SEQ ID NO: 93 |
| SYDSSNWVF | λ | SEQ ID NO: 94 |
| SYDSSNRVF | λ | SEQ ID NO: 95 |
| SYDSNNHWVF | λ | SEQ ID NO: 96 |
| SYDSSNHWVF | λ | SEQ ID NO: 97 |
| SYDSSDHWVF | λ | SEQ ID NO: 98 |
| SYDSGNWVF | λ | SEQ ID NO: 99 |
| SYDSNNWVF | λ | SEQ ID NO: 100 |
| SYDSSNRVF | λ | SEQ ID NO: 101 |
| YDSSNHWVFG | λ | SEQ ID NO: 102 |
| SYDSSNHWVF | λ | SEQ ID NO: 103 |
| SYDSSNHWVF | λ | SEQ ID NO: 104 |
| SYDSSNRVF | λ | SEQ ID NO: 105 |
| SYDSSNRVF | λ | SEQ ID NO: 106 |
| SYDSSVRVF | λ | SEQ ID NO: 107 |
| SYDSSNWVF | λ | SEQ ID NO: 108 |
| SYDSSNHWVF | λ | SEQ ID NO: 109 |
| SFDSSNRVF | λ | SEQ ID NO: 110 |
| SYDRSNRVF | λ | SEQ ID NO: 111 |
| SYDSSNHWVF | λ | SEQ ID NO: 112 |
| SYDSSNHWVF | λ | SEQ ID NO: 113 |
| SYDSSNHWVF | λ | SEQ ID NO: 114 |
| SFDNSNRVF | λ | SEQ ID NO: 115 |
| SYDSSNRVF | λ | SEQ ID NO: 116 |
| SYDSNNRVF | λ | SEQ ID NO: 117 |

TABLE 2
anti-CD3 antibody Variable Region Sequences

| Antibody | HC Sequence | | LC Sequence | |
|---|---|---|---|---|
| 70701_05 | EVQLVESGGGLVKPG | SEQ | NFMLTQPHSVSESPG | SEQ |
| C15A | GSLRLSCAASGFTFS | ID | KTVTISCTRSSGSIA | ID |
| | RYSMNWVRQTPGKGL | NO: | SNYVQWYQQRPGSSP | NO: |
| | EWVSSISNSCNYIYY | 118 | TTVVYEDNQRPSGVP | 176 |
| | ADSVKGRFTISRDSA | | DRFSGSIASSSNSAS | |
| | KNSLYLQMNSLRAED | | LTISGLKTEDEADYY | |
| | TAVYYCARMRYNYFD | | CQSYDSSNVVFGGGT | |
| | YWGQGTLVTVSS | | KLTVL | |
| 70701_05 | EVQLVESGGGLVQPG | SEQ | SYELTQPPSVSVSPG | SEQ |
| D06A | GSLRLSCVASGFTFS | ID | QTASITCSGDKLGHK | ID |
| | NYWMNWVRQAPGKGL | NO: | YVCWYQQKPGQSPVL | NO: |
| | EWVANIKKDGSEKYY | 119 | VIYQDNKRPSGIPER | 177 |
| | VDSVKGRFTISRDNA | | FSGSNSGNTATLTIS | |
| | KNSLYLQMNSLRAED | | GTQAMDEADYYCQAW | |
| | TAVYYCARVKWDLLD | | DSSTHVVFGGGTKLT | |
| | YWGQGTLVTVSS | | VL | |
| 70701_05 | EVQLVESGGGLVKPG | SEQ | NVMLIQPHSVSESPG | SEQ |
| L17A | GSLRLSCAASGFNLS | ID | KTVTISCTRSSGSIA | ID |
| | TYSMNWVRQAPGKGL | NO: | SKYVQWYQQRPGSSP | NO: |
| | EWVSSISRSSSYIYY | 120 | TTVIYEDNQRPSGVP | 178 |
| | ADAVKGRFTISRDNA | | DRFSGSIDSSSNSAS | |
| | QNSLYLQMNSLRAED | | LTISGLKTEDEADYY | |
| | TAVYYCAREQWEPLY | | CQSYDSSNRVFGGGT | |
| | FDYWGQGTLVTVSS | | KLTVL | |
| 70701_05 | QITLKESGPTLVKPT | SEQ | QSALTQSASVSGSPG | SEQ |
| M03A | QTLTLTCSFSGFSFT | ID | QSITISCTGTSSDVG | ID |
| | TSGVGVGWIRQPPGK | NO: | SYNLVSWYQQHPGKA | NO: |
| | ALECLALIYWNDDKR | 121 | PKLMIYEGSKRPSGV | 179 |
| | YSPSLKNRLTITKDT | | SNRFSGSKSGNTASL | |
| | SKNQVVLTMTNMDPV | | TISGLQAEDEADYYC | |
| | DTATYFCAHLAHWGP | | CSYAGSSTLIFGGGT | |
| | YFDYWGQGTLVTVSS | | KLTVL | |
| 70701_06 | EVLLVESGGGLVKPG | SEQ | SYVLTQPPSVSVAPG | SEQ |
| A03A | GSLRLSCEASGFTFS | ID | QTASITCGGNNIGSK | ID |
| | RYSMNWVRQAPGKGL | NO: | SVHWYQQKPGQAPVL | NO: |
| | EWVSSISRSSRYIYY | 122 | VVYDDTDRPSGIPER | 180 |
| | ADSVKGRFTISRDNA | | FSGSNSGNTATLTIS | |
| | KNSLYLQMISLRAED | | RVEAGDEADYYCQVW | |
| | TAVFYCARFSNWGSG | | DSSSDHPVFGGGTKL | |
| | GYFDYWGQGTLVTVS | | TVL | |
| | S | | | |
| 70701_06 | EVQLVESGGGLVKPG | SEQ | NFMLTQPHSVSESPG | SEQ |
| A16A | GSLRLSCAASGFTVN | ID | KTVTISCTRSSGSIA | ID |
| | NYSMNWVRQAPGKGL | NO: | SNYVQWYQQRPGSSP | NO: |
| | EWVSSISRSSNYIYY | 123 | TPVIYEDYQRPSGVP | 181 |
| | ADSVKGRFTISRDNA | | DRFSGSIDSSSNSAS | |
| | KNSLYLQMNSLRAED | | LTISGLKTEDEADYY | |
| | TAVYYCARERWALLF | | CQSFDRSNRVFGGGT | |
| | FEYWGQGTLVTVSS | | KLTVL | |
| 70701_06 | EVQLVESGGGLVKPG | SEQ | NVMLIQPHSVSESPG | SEQ |
| B15A | GSLRLSCAASGFNLS | ID | KTVTISCTRSSGSIA | ID |
| | TYSMNWVRQAPGKGL | NO: | SKYVQWYQQRPGSSP | NO: |
| | EWVSSISRSSSYIYY | 124 | TTVIYEDNQRPSGVP | 182 |
| | ADSVKGRFTISRDNA | | DRFSGSIDSSSNSAS | |
| | QNSLYLQMNSLRAED | | LTISGLKTEDEADYY | |
| | TAVYYCAREKWELLY | | CQSYDSSNRVFGGGT | |
| | FDYWGQGTLVTVSS | | KLTVL | |
| 70701_06 | EVQLVESGGGLVKPG | SEQ | NFMLTQPHSVSESPG | SEQ |
| M17A | GSLRLSCAASGFTFS | ID | KTVTISCTRSSGNIA | ID |
| | SYSMNWVRQAPGKGL | NO: | RNYVQWYQQRPGSSP | NO: |
| | EWVASMSRSSSYIYY | 125 | TTVIYEDNQRPSGVP | 183 |
| | AHSVKGRFTISRDNA | | DRFSGSIDSSSNSAS | |
| | KNSLYLQLNSLRAED | | LTISGLKTEDEADYY | |
| | TAVYYCARERWELLF | | CQSYDSSNRVFGGGT | |
| | FDYWGQGTLVTVSS | | KLTVL | |
| 70701_07 | EVQLVESGGGLVKPG | SEQ | NFMLTQPHSVSESPG | SEQ |
| N16A | GSLRLSCAASGFTLS | ID | KTVTISCTRSSGSIA | ID |
| | SYNMDWVRQAPGKGL | NO: | SNFVQWYQQRPGSSP | NO: |
| | EWVSSISRSSNYIYY | 126 | TTVIYEDNQRPSGVP | 184 |
| | ADSVKGRFTISRDNA | | DRFSGSIDSSSKSAS | |
| | ENSLYLQMNSLRAED | | LTISGLKTEDEADYY | |
| | TAVYYCAREKWVQLY | | CQSYDSSNRVFGGGT | |
| | FDFWGQGTLVTVSS | | KLTVL | |
| 70701_08 | EVQLVESGGGLVKPG | SEQ | SYVLTQPPSVSVAPG | SEQ |
| J03A | GSLRLSCAASGFTFR | ID | QTARITCGGNNIGSK | ID |
| | SYSMNWVRQAPGKGL | NO: | SVHWYQQKPGQAPVL | NO: |
| | EWVSSISRSSSYIYY | 127 | VVYDDSDRPSGIPER | 185 |
| | ADSVKGRFTISRDNA | | FSGSNSENTATLTIS | |
| | KNSLYLQMNSLRAED | | RVEAGDEADYYCQVW | |
| | TAVYYCARFSNWGSG | | DSTSDHPVFGGGTKL | |
| | GYFDYWGQGTLVTVS | | TVL | |
| | S | | | |
| 70701_09 | EVQLVESGGGLVKPG | SEQ | NFMLTQPHSVSESPG | SEQ |
| C02A | GSLRLSCAASGFTFS | ID | KTVTISCTRSSGSIA | ID |
| | RYSMNWVRQTPGKGL | NO: | SNYVQWYQQRPGSSP | NO: |
| | EWVSSISSSSNYIYY | 128 | TTVVYEDNQRPSGVP | 186 |
| | ADSVKGRFTISRDNA | | DRFSGSIASSSNSAS | |
| | KNSLYLQMNSLRAED | | LTISGLKTEDEADYY | |
| | TAVYYCARMRYNYFD | | CQSYDSSNVVFGGGT | |
| | YWGQGTLVTVSS | | KLTVL | |
| 70701_09 | EVQLVESGGGLGQPG | SEQ | NFMLTQPHSVSESPG | SEQ |
| K17A | GSLRLSCAASGFTFS | ID | KTVTFSCTRSSGSIA | ID |
| | SYSMNWVRQAPGKGL | NO: | SNYVQWYQQRPGSSP | NO: |
| | EWVSYISRSSSTIYY | 129 | TTVIYVDNQRPSGVP | 187 |
| | ADSVKGRFIISRDKA | | DRFSGSIDSSSNSAS | |
| | KNSLYLQMNSLRDED | | LTISGLKTEDEADYY | |
| | TAVYYCARVVRDYGM | | CQSYDSSNHWVFGGG | |
| | DVWGQGTTVTVSS | | TKLTVL | |
| 70701_10 | EVQLVESGGGLVKPG | SEQ | NFMLTQPHSVSESPG | SEQ |
| D02A | GSLRLSCAASGFTLS | ID | KTVTISCTRSSGSIA | ID |
| | SYSMNWVRQAPGKGL | NO: | SNYVQWYQQRPGSSP | NO: |
| | EWVSTMSRSSSYIYY | 130 | TTVIYEDNQRPSGVP | 188 |
| | ADSVKGRFTISRDNA | | DRFSGSIDSSSNSAS | |
| | KNSLYLQMNSLRAED | | LTISGLKTEDEADYY | |
| | TAVYYCARERWELLF | | CQSYDSSNRVFGGGT | |
| | FDYWGQGTLVTVSS | | KLTVL | |
| 70701_10 | EVQLVESGGGLVKPG | SEQ | NVMLTQTHSVSESPG | SEQ |
| F19A | GSLRLSCAASGFTIS | ID | KTVTISCTRSSGSIA | ID |
| | RYSMNWVRQAPGKGL | NO: | SNYVQWYQQRPGSSP | NO: |
| | EWVSSISRSSSHIYY | 131 | TTVIYEDNQRPSGVP | 189 |
| | ADSVKGRFTISRDNA | | DRFSGSIDSSSNSAS | |
| | KNSMYLQMNSLRAED | | LTISGLKTEDEADYY | |
| | TAVYYCAREKWELLY | | CQSYDSSNRVFGGGT | |
| | FDYWGQGTLVTVSS | | KLTVL | |
| 70701_10 | EVQLVESGGGLVKPG | SEQ | NFMLTQPHSVSESPG | SEQ |
| J09A | GSLRLSCAASGFTLS | ID | KTVTISCTRSSGSIA | ID |
| | SYSMDWVRQAPGKGL | NO: | SNFVQWYQQRPGSSP | NO: |
| | EWVSSISRSSNYIYY | 132 | TTVIYEDNQRPSGVP | 190 |
| | ADSVKGRFTISRDNA | | DRFSGSIDSSSNSAS | |
| | ENSLYLQMNSLRAED | | LTISGLKTEDEADYY | |
| | TAVYYCAREKWVQLY | | CQSYDSSNRVFGGGT | |
| | FDFWGQGTLVTVSS | | KLTVL | |
| 70701_10 | EVQLVEFGGGLVRPG | SEQ | NFMLTQPHSVSESPG | SEQ |
| N20A | GSLRLSCVASGFTFS | ID | KTVTISCTRSSGSIA | ID |
| | IFTMNWVRQAPGKGL | NO: | SNYVQWYQQRPGSSP | NO: |
| | EWVSTSSRSTSYIYY | 133 | TTVIYEDNQRPSGVP | 191 |
| | ADSVKGRFTISRDNA | | DRFSGSIDSSSNSAS | |
| | KNSLYLQMNSLRAED | | LTISGLKTEDEADYY | |
| | TAVYYCAREQWELLY | | CQSYDSSNRVFGGGT | |
| | FDYWGQGTLVTVSS | | KLTVL | |
| 70701_11 | QVQLVQSGAEVKKPG | SEQ | QSVLTQPPSVSAAPG | SEQ |
| B10A | SSVKVSCKASGGTFS | ID | QKVTISCSGSSSNIG | ID |
| | SYAISWVRQAPGQGL | NO: | NNYVSWYQQLPGTAP | NO: |
| | EWMGGIIPIFATANY | 134 | KLLIYDNYKRPSGIP | 192 |
| | AQKFQGRVTITADKS | | DRFSGSKSGTSATLG | |
| | TSTAYMELSSLRSED | | ITGLQTGDEADYYCG | |
| | TAVYYCARGGVGRNY | | TWDSSLSAVVFGGGT | |
| | YYYGMDVWGQGTTVT | | KLTVL | |
| | VSS | | | |
| 70701_11 | EVQLVESGGGLVQPG | SEQ | SYELTQPPSVSVSPG | SEQ |
| K23A | GSLRLSCAASGFTFS | ID | QTARITCSGDALPKQ | ID |
| | SYGMNWVRQAPGKGL | NO: | YAYWYQQKPGQAPVL | NO: |
| | EWVSYISRSSRTRYY | 135 | VIYKDSERPSGIPER | 193 |
| | ADSVKGRFTISRDNA | | FSGSSSGTTVTLTIS | |
| | KNSLYLQMNSLRDED | | GVQAEDEADYYCQSA | |
| | TSVYYCARDGRWELL | | DTSGTYRVFGGGTKL | |
| | AFDIWGQGTMVTVSS | | TVL | |
| 70701_11 | EVQLVESGGGLVKPG | SEQ | NFMLTQPHSVSESPG | SEQ |
| L01A | GSLRLSCAASGFTFS | ID | KTVTISCTRSSGSIA | ID |
| | RYSINWVRQAPGKGL | NO: | SNYVQWYQQRPGSSP | NO: |
| | EWVSSISSSSSYIYY | 136 | TTVIYEDNQRPSGVP | 194 |
| | ADSLKGRFTISRDNA | | DRFSGSIDSSSNSAS | |
| | RNSLYLQMNSLRAED | | LTISGLKTEDEADYY | |
| | TAVYYCAKMRYNYFD | | CQSYDGSNVVFGGGT | |
| | YWGQGALVTVSS | | KLTVL | |
| 70701_11 | EVQLVESGGGLVKPG | SEQ | NFMLTQPHSVSESPG | SEQ |
| M11A | GSLRLSCAASGFTFS | ID | KTVTISCTRSSGSIA | ID |
| | RYSINWVRQAPGKGL | NO: | SNYVQWYQQRPGSSP | NO: |
| | EWVSSISSSSSYIYY | 137 | TTVIYEDNQRPSGVP | 195 |
| | ADSLKGRFTISRDNA | | DRFSGSIDSSSNSAS | |
| | RNSLYLQMNSLRAED | | LTISGLKTEDEADYY | |
| | TAVYYCAKMRYNYFD | | CQSYDGSNVVFGGGT | |
| | YWGQGALVTVSS | | KLTVL | |
| 70701_12 | EVQLVEFGGGLVRPG | SEQ | NFMLTQPHSVSESPG | SEQ |
| F16A | GSLRLSCVASGFTFS | ID | KTVTISCTRSSGSIA | ID |
| | IFTMNWVRQAPGKGL | NO: | SNYVQWYQQRPGSSP | NO: |
| | EWVSTSSRSTSYIYY | 138 | TTVIYEDNQRPSGVP | 196 |
| | ADSVKGRFTISRDNA | | DRFSGSIDSSSNSAS | |
| | KNSLYLQMNSLRAED | | LTISGLKTEDEADYY | |
| | TAVYYCAREQWELLY | | CQSYDSSNRVFGGGT | |
| | FDYWGQGTLVTVSS | | KLTVL | |
| 70701_12 | EVQLVESGGGLVKPG | SEQ | NFMLTQPHSVSESPG | SEQ |
| J12A | GSLRLSCAASGFTFS | ID | KTVTISCTRSSGSIA | ID |
| | RYSINWVRQAPGKGL | NO: | SNYVQWYQQRPGSSP | NO: |
| | EWVSSSSRSSSYIYY | 139 | TTVIYEDNQRPSGVP | 197 |
| | ADSVKGRFTISRDNA | | DRFSGSIDSSSNSAS | |
| | KNSLYLQMNSLRAED | | LTISGLKTEDEADYY | |
| | TAVYYCAREQWELLY | | CQSYDRSNRVFGGGT | |
| | FDYWGQGTLVTVSS | | KLTVL | |
| 70701_12 | EVQLVESGGGLVKPG | SEQ | NFMLTQPHSVSESPG | SEQ |
| O16A | GSLRLSCGASGFTFS | ID | KTVTISCTRSSGNIA | ID |
| | SYSMNWVRQAPGKGL | NO: | SNYVQWYQQRPGSSP | NO: |
| | EWVSSISSSSRYIYY | 140 | TTVIYEDNQRPSGVP | 198 |
| | ADSLRGRFTISRDNA | | DRFSGSIDSSSNSAS | |
| | KNSLYLQMNSLRAED | | LTISGLKTEDEADYY | |
| | TAVYYCARMRYNYFD | | CQSYDSSNVVFGGGT | |
| | YWGQGTLVTVSS | | KLTVL | |
| 70701_13 | EVQLVESGGGLVKPG | SEQ | NFMLTQPHSVSESPG | SEQ |
| C01A | GSLRLSCAASGFTFS | ID | KTVTISCTRSSGSIA | ID |
| | RYSMNWVRQAPGKGL | NO: | NNFVQWFQQRPGSSP | NO: |
| | EWVSSISGSSRYIYY | 141 | TDVIYEDNQRPSGVP | 199 |
| | ADSVKGRFTVSRDNA | | DRFSGSIDSSSNSAS | |
| | KNSLYLQMSSLSPED | | LTISGLKTEDEADYY | |
| | TAVYYCARERYNYFD | | CQSYDSSNRVFGGGT | |
| | YWGQGTLVTVSS | | KLTVL | |
| 70701_13 | EVQLVESGGGLVKPG | SEQ | NFMLTQPHSVSESPG | SEQ |
| G21A | GSLRLSCAASGFTFS | ID | KTVTISCTRSSGSIA | ID |
| | IYSMNWVRQAPGKGL | NO: | SNYVQWYQQRPGSSP | NO: |
| | EWVSSISKTSTFIFY | 142 | TTVIYEDNQRPSGVP | 200 |
| | ADSVKGRFTISRDNA | | DRFSGSIDSSSNSAS | |
| | KNSLYLQMNSLSAED | | LTISGLKTEDEADYS | |
| | TAVYYCARERYNYFD | | CQSYNGNNRVFGGGT | |
| | YWGQGTLVTVSS | | KLTVL | |
| 70701_13 | EVQLVESGGGLVKPG | SEQ | NFMLTQPHSVSESPG | SEQ |
| H15A | GSLRLSCAASGFTFS | ID | KTVTISCTRSSGSIA | ID |
| | RYSLNWVRQAPGKGL | NO: | SNYVQWYQQRPGSSP | NO: |
| | EWVSSISSSSRYIFY | 143 | TPVIYEDNQRPSGVP | 201 |
| | ADSVQGRFTISRDNA | | DRFSGSIDSSSNSAS | |
| | KNSLYLQMNSLRAED | | LTISGLKTEDEADYF | |
| | TAVYFCARERYNYFD | | CQSYDNSNRVFGGGT | |
| | YWGQGTLVTVSS | | KLTVL | |
| 70701_14 | EVQLVESGGGLVKPG | SEQ | NFMLTQPHSVSESPG | SEQ |
| F08A | GSLRLSCAASGFTFS | ID | KTVTISCTRSSGNIA | ID |
| | RYSMNWVRQAPGKGL | NO: | SNYVQWYQQRPGSSP | NO: |
| | EWVSSISSSSRYIFY | 144 | TAVIYEDNQRPSGVP | 202 |
| | ADSVKGRFTISRDNA | | DRFSGSIDSSSNSAS | |
| | KNSLYLQMNSLRAED | | LTISGLKTEDEADYY | |
| | TAVYYCARERYNYFD | | CQSYDGSNRVFGGGT | |
| | FWGQGTLVTVSS | | KLTVL | |
| 70701_14 | EVQLVESGGGLVKPG | SEQ | NFMLTQPHSVSESPG | SEQ |
| F21A | GSLRLSCAASGFTFS | ID | KTVTISCTRSSGSIA | ID |
| | RYSMNWVRQAPGKGL | NO: | SNYVQWYQQRPGSSP | NO: |
| | EWVSSISSSSRYIFY | 145 | TTVIYEDNQRPSGVP | 203 |
| | ADSVKGRFTMSRDNA | | DRFSGSIDSSSNSAS | |
| | KNSLYLQMNNLRAED | | LTISGLKTEDEADYY | |
| | TAVYYCAREPYNYFD | | CQSYDNSNRVFGGGT | |
| | YWGQGTLVSVSS | | KLTVL | |
| 70701_14 | EVHLVESGGGLVKPG | SEQ | NFMLTQPHSVSESPG | SEQ |
| H05A | GSLRLSCAASGFTFS | ID | KTVTISCTRSSGSIA | ID |
| | RYSMNWVRQAPGKGL | NO: | SNYVQWYQQRPGSSP | NO: |
| | EWVSSISSSSRYIYY | 146 | TTVIYEDNQRPSGVP | 204 |
| | ADSVKGRFTISRDNA | | DRFSGSIDSSSNSAS | |
| | KNSLYLQMNSLRAED | | LTISGLKTEDEADYY | |
| | TAVYYCARERYNYFD | | CQSYDSSNRVFGGGT | |
| | YWGQGTLVTVSS | | KLTVL | |
| 70701_14 | EVQLVESGGGLVKPG | SEQ | NFMLTQPHSVSESPG | SEQ |
| K01A | GSLRLSCAASGFTFS | ID | KTVTISCTRSSGSIA | ID |
| | RYSLNWVRQAPGKGL | NO: | SNYVQWYQQRPGSSP | NO: |
| | EWVSSISSSSRYIYY | 147 | TTVIYEDYQRPSGVP | 205 |
| | ADSVKGRFTISRDNA | | ARFSGSIDSSSNSAS | |
| | KNSLYLQMNSLRAED | | LTISGLKTEDEADYY | |
| | TAVYYCARERYNYFD | | CQSYDNNNRVFGGGT | |
| | YWGQGTLVTVSS | | MLTVL | |
| 70701_14 | EVQLVESGGGLVKRG | SEQ | DFMLTQPHSVSESPG | SEQ |
| K02A | GSLRLSCATSGFTLS | ID | KTVTISCTRSSGSIA | ID |
| | RYSMNWVRQAPGKGL | NO: | SNYVQWYQQRPGSSP | NO: |
| | EWVSSISSSSSYIYY | 148 | TAVIYEDNQRPSGVP | 206 |
| | ADSVKGRFTISRDNA | | DRFSGSIDSSSNSAS | |
| | KNSLYLQMNSLRAED | | LTISGLKTEDEADYY | |
| | TALYYCARERYNYFD | | CQSYDNSNRVFGGGT | |
| | YWGQGTLVTVSS | | KLTVL | |
| 70701_14 | EVQLVESGGGLVKPG | SEQ | NFMLTQPHSVSESPG | SEQ |
| M09A | GSLRLSCAASGFTFS | ID | KTVTISCTRSSGSIA | ID |
| | IYSMNWVRQAPGKGL | NO: | SNYVQWYQQRPGSSP | NO: |
| | EWVSSISKSSRYIFY | 149 | TTVIYEDYQRPSGVP | 207 |
| | ADSVKGRFTISRDNA | | DRFSGSIDSSSNSAS | |
| | KNSLYLQMNSLSAED | | LTISGLKTEDEADYS | |
| | TAVYYCARERYNYFD | | CQSYDGNNRVFGGGT | |
| | SWGQGTLVTVSS | | KLTVL | |
| 70701_16 | EVQLVESGGGSVQPG | SEQ | NFMLTQPHSVSESPG | SEQ |
| D18A | GSLRLTCAASGFTFS | ID | KTVTISCTRSSGSIA | ID |
| | TYSMNWVRQAPGKGL | NO: | SNYVQWYQQRPGSSP | NO: |
| | EWVSYISRSSSTIYY | 150 | TTVIYDDNQRPSGVP | 208 |
| | ADSVKGRFTISRDNA | | DRFSGSIDSSSNSAS | |
| | KNSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_16 | EVQLVESGGGLVQPG | SEQ | NFMLTQPHSVSESPG | SEQ |
| E13A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISRSTSTIYY | 151 | TTVIYDDNQRPSGVP | 209 |
| | ADSVKGRFTISRDNA | | DRFSGSIDSSSNSAS | |
| | KNSLYLQMNSLRDED | | LTISGLKTEDEAEYY | |
| | TAVYYCARVRRNYGM | | CQSYDSSNHWVFGGG | |
| | DVWGQGTTITVSS | | TKLTVL | |
| 70701_16 | EVQLVESGGGLVQPG | SEQ | NFMLTQPHSVSESPG | SEQ |
| L13A | GSLRLSCAASGFTFS | ID | KTVTISCTRSSGSIA | ID |
| | SYSMNWVRQAPGKGL | NO: | SNYVQWFQQRPGSSP | NO: |
| | EWVSYISRSSSTIYY | 152 | TTVIYDDNQRPSGVP | 210 |
| | ADSVKGRFTISRDNA | | DRFSGSIDSSSNSAS | |
| | KNSLYLQMNSLRDED | | LTISRLKTEDEADYY | |
| | TAVYYCARVRRNYGM | | CQSYDSSNWVFGGGT | |
| | DVWGQGTTVTVSS | | KLTVL | |
| 70701_16 | EVQLVESGGGLVKPG | SEQ | NFMLTQPHSVSESPG | SEQ |
| P13A | GSLRLSCAASGFTFS | ID | KTVTISCTRSSGSIA | ID |
| | SYSMNWVRQAPGKGL | NO: | SNYVQWYQQRPGSSP | NO: |
| | EWVSSSSRSSSYIYY | 153 | TTVIYEDNQRPSGVP | 211 |
| | ADSVKGRFTITRDNA | | DRFSGSIDSSSNSAS | |
| | KNSLYLQMNSLRAED | | LTISGLKTEDEADYY | |
| | TAVYYCARERWELLY | | CQSYDSSNRVFGGGT | |
| | FDYWGQGTLVTVSS | | KLTVL | |
| 70701_17 | EVQLVESGGGLVQPG | SEQ | NFMLTQPHSVSESPG | SEQ |
| A23A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISRSSNTIYY | 154 | TTVIYDDNQRPSGVP | 212 |
| | ADSVKGRFTISRDNA | | DRFSGSIDSSSNSAS | |
| | RNSLYLQMSSLRDED | | LTISGLKTEDEADYY | |
| | AAVYYCARVRRNYGM | | CQSYDSNNHWVFGGG | |
| | DVWGQGTTVTVSS | | TKLTVL | |
| 70701_17 | EVQLVESGGGLVQPG | SEQ | NFMLTQPHSVSESPG | SEQ |
| G08A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISSSGSTIYY | 155 | TTVIYDDNQRPSGVP | 213 |
| | ADSVKGRFTISRDNA | | DRFSGSIDSSSSSAS | |
| | KSSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_17 | EVQLVESGGGLVQPG | SEQ | KLMLTQPHSVSESPG | SEQ |
| H17A | GSLRLSCVVSGFTFS | ID | KTVTISCTRSSGSIA | ID |
| | SYSMNWVRQAPGKGL | NO: | RNYVQWYQQRPGSSP | NO: |
| | EWVSYISSSHSTIYY | 156 | TTVIYDDNQRPSGVP | 214 |
| | ADSVKGRLTISRDNA | | DRFSGSIDSSSNSAS | |
| | KNSLYLQMNSLRDED | | LTISGLRTEDEADYY | |
| | TAVYYCARVRRNYGM | | CQSYDSSDHWVFGGG | |
| | DVWGQGTTVTVSS | | TKLTVL | |
| 70701_18 | EVQLVESGGGLVQPG | SEQ | NFMLTQPHSVSESPG | SEQ |
| A16A | GSLRLSCAASGFTFS | ID | KTVTISCTRSSGNIA | ID |
| | SYSMNWVRQAPGKGL | NO: | RNYVQWYQQRPGSSP | NO: |
| | EWVSYISSSINTIYY | 157 | TTVIYEDNQRPSGVP | 215 |
| | ADSVKGRFTISRDNA | | DRFSGSIDSSSNSAS | |
| | KNSLYLQMNSLRDED | | LTISGLKTEDEADYY | |
| | TAVYYCARVRRNYDM | | CQSYDSGNWVFGGGA | |
| | DVWGQGTTVTVSS | | KLTVL | |
| 70701_18 | EVQLVESGGGLVQPG | SEQ | NFMLTQPHSVSESPG | SEQ |
| E19A | GSLRLSCAASGFTFS | ID | KTVTISCTRSSGSIA | ID |
| | SYSVNWVRQAPGKGL | NO: | SNYVQWYQQRPGSSP | NO: |
| | EWVSYISRSSSIIYY | 158 | TTVIYDDNQRPSGVP | 216 |
| | VDSVKGRFTISRDNA | | DRFSGSIDSSSNSAS | |
| | KNSLYLQMNSLRDED | | LTISGLKTEDEADYY | |
| | TAVYYCARVRRNYGM | | CQSYDSNNWVFGGGT | |
| | DVWGQGTTVTVSS | | KLTVL | |
| 70701_18 | EVQLVESGGGLVKPG | SEQ | NFMLTQPHSVSESPG | SEQ |
| M14A | GSLRLSCAASGFILN | ID | KTITISCTRRNGNIA | ID |
| | NYSMNWVRQAPGKGL | NO: | SNYVQWYQQRPGRSP | NO: |
| | EWVSSISRSSSYIYY | 159 | TTVIYEDNQRPSGVP | 217 |
| | ADSVKGRFTISRDNA | | DRFSGSIDSSSNSAS | |
| | KNSLYLQMNSLRADD | | LTISGLKTEDEADYY | |
| | TAVYYCAREQWVLLY | | CQSYDSSNRVFGGGT | |
| | FDYWGQGTLVTVSS | | KLTVL | |
| 70701_19 | EAQLVESGGGLVQPG | SEQ | HFMLTQPHSVSESPG | SEQ |
| A10A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISRSSSTIYY | 160 | TTVIYDDNQRPSGVP | 218 |
| | ADSAKGRFTISRDNA | | DRFSGSIDSSSNSAS | |
| | KNSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_19 | EVQLVESGGGLVQPG | SEQ | NFMLTQPHSVSESPG | SEQ |
| C22A | ESLRLSCAASGFTFS | ID | KTVTISCTRSSGSIA | ID |
| | SYSMNWVRQAPGKGL | NO: | SNYVQWYQQRPGSSP | NO: |
| | EWVSYISRSSNTIYY | 161 | TTVIYDDNQRPSGVP | 219 |
| | ADSVKGRFTISRDNA | | DRFSGSIDSSSNSAS | |
| | RNSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_19 | EVQLVESGGGLVQPG | SEQ | NFMLTQPHSVSESPG | SEQ |
| D03A | GSLRLSCAVSGFTFS | ID | KTVTISCTRSSGSIA | ID |
| | SYSMNWVRQAPGKGL | NO: | TNYVQWYQQRPGSSP | NO: |
| | EWVSYSSSSSSTIYY | 162 | TTLIYDDNQRPSGVP | 220 |
| | ADSVKGRFTISRDNA | | DRFSGSIDSSSNSAS | |
| | KNSVYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_19 | EVHLVESGGGLVKPG | SEQ | NFMLTQPHSVSESPG | SEQ |
| E13A | GSLRLSCAASGITFS | ID | KTVTISCTRSSGSIA | ID |
| | RYSMNWVRQAPGKGL | NO: | SNYVQWYQQRPGSSP | NO: |
| | EWVSSISRSSNYIYY | 163 | TTVIYEDNQRPSGVP | 221 |
| | ADSVKGRFTISRDNA | | DRFSGSIDSSSNSAS | |
| | KNSLYLQMNSLRAED | | LTISGLKTEDEADYY | |
| | TAVYYCARERWELLY | | CQSYDSSNRVFGGGT | |
| | FDYWGQGTLVTVSS | | KLTVL | |
| 70701_19 | QVQLQESGPGLVKPS | SEQ | NFMLTQPHSVSESPG | SEQ |
| I16A | QTLSLTCTVSGGSIT | ID | KTVTISCTRSSGSIA | ID |
| | SGASYWSWIRQHPGK | NO: | SNYVQWYQQRPGSSP | NO: |
| | GLEWIGYIYYSGSTY | 164 | TTVIYEDSQRPSGVP | 222 |
| | HSPSLKSRVTISVDT | | DRFSGSIDSSSNSAS | |
| | SKNRFSLMLSSVTAA | | LTISGLKTEDEADYY | |
| | DTAVYYCARERKYPL | | CQSYDSSNRVFGGGT | |
| | QFDYWGQGTLVTVSS | | KVTVL | |
| 70701_19 | QVQLQESGPGLMKPS | SEQ | NFMLTQPHSVSESPG | SEQ |
| J10A | QTLSLTCTVSGGSIS | ID | KTVTISCTRSSGSIA | ID |
| | SGVYYWNWIRQHPGK | NO: | SNYVQWFQQRPGSSP | NO: |
| | GLEWIGDIYYSGSTY | 165 | TTVIFEDNQRPSGVP | 223 |
| | YNPSLKSRVTISVDT | | DRFSGSIDSSSNSAS | |
| | SKNQFSLKLSSVTAA | | LTISGLKTEDEADYY | |
| | DTAVYYCVRERKYPL | | CQSYDSSVRVFGGGT | |
| | YFDYWGQGTLVTVSS | | KLTVL | |
| 70701_19 | EVQLVESGGGLVQPG | SEQ | NFVLPQPHSVSESPG | SEQ |
| O15A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISSSSSTIYY | 166 | TSVIYDDSQRPSGVP | 224 |
| | ADSVKGRFTISRDNA | | DRFSGSIDSSSNSAS | |
| | KNSLFLQMNSLRDED | | LTISGLTTEDEADYY | |
| | TAVYYCARVRRNYGM | | CQSYDSSNWVFGGGT | |
| | DVWGQGTTVTVSS | | KLTVL | |
| 70701_20 | EVQLVESGGGLVQPG | SEQ | NFMLTQPHSVSESPG | SEQ |
| O18A | GSLRLSCAASGLTFS | ID | KTVTISCTRSSGSIA | ID |
| | RYSMNWVRQAPGKGL | NO: | SNYVQWYQQRPGSSP | NO: |
| | YWVSYISTSSTTIYY | 167 | TTVIYDDNQRPSGVP | 225 |
| | ADSVKGRFTISRDNA | | DRFSGSIDSSSNSAS | |
| | KNSLSLQMNSLRDED | | LTISGLKTEDEADYY | |
| | TAVYYCARVRRNYGM | | CQSYDSSNHWVFGEG | |
| | DVWGQGTTVTVSS | | TKLTVL | |
| 70701_21 | QVQLQESGPGLVEPS | SEQ | NFMLTQPHSVSESPG | SEQ |
| A06A | QTLSLICTVSGGSIS | ID | KTVTISCTRSSGSIA | ID |
| | SGASYWSWIRQHPGK | NO: | SNYVQWYQLRPGSSP | NO: |
| | GLEWIGYIYYSGTTY | 168 | TTVIYEDNQRPSGVP | 226 |
| | FNPSLKSRVTISLDT | | DRFSGSIDSSSNSAS | |
| | SKNQFSLKLSSVTAA | | LTISGLKTEDEADYY | |
| | DTAVYYCARERKYPL | | CQSFDSSNRVFGGGT | |
| | QFDYWGQGTLVTVSS | | KLTVL | |
| 70701_21 | QVQLQESGPGLVKPS | SEQ | NFMLTQPHSVSESPG | SEQ |
| J18A | QTLSLTCTVSGGSIS | ID | KTVTISCTRSSGSVA | ID |
| | SGAYCWSWIRQHPGK | NO: | SNYVQWYQQRPGSSP | NO: |
| | GLEWIGSIYYSGSTY | 169 | TTVIYEDYQRPSGVP | 227 |
| | YNPSLKSRVTISVDT | | DRFSGSIDSSSNSAS | |
| | SKKQFSLKLSSVTAA | | LTISGLKTEDEADYY | |
| | DTAVYYCARERKYLL | | CQSYDRSNRVFGGGT | |
| | YFDYWGQGTLVTVSS | | KLTVL | |
| 70701_22 | EMQLVESGGGLVQRG | SEQ | YFMLTQPHSVSESPG | SEQ |
| D03A | GSLRLSCAASKFTFS | ID | KTVTISCTRSSGSIA | ID |
| | GYAMNWVRQAPGKGL | NO: | SNYVQWYQQRPGSSP | NO: |
| | EWVSYISSSTSTTYY | 170 | TTVIYDDNQRPSGVP | 228 |
| | ADSVKGRFTISRDNA | | DRFSGSIDSSSNSAS | |
| | KNSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_22 | EVQLVESGGGLVQPG | SEQ | NFMLTQPHSVSESPG | SEQ |
| O09A | GSLRLSCAASGFTFS | ID | KTVTISCTRSSGSIA | ID |
| | RYSINWVRQAPGKGL | NO: | SNYVQWYQQRPGSSP | NO: |
| | EWVSYISSNTSTIYY | 171 | TTVIYDDNQRPSGVP | 229 |
| | ADSVKGRFTISRDNA | | DRFSGSIDSSSNSAS | |
| | KNSLYLQMNSLRDED | | LTISGLKTEDEADYY | |
| | TAVYYCARVRRNYGM | | CQSYDSSNHWVFGGG | |
| | DVWGQGTTVTVSS | | TKLTVL | |
| 70701_23 | EVQLVGSGGGLVQPG | SEQ | NFMLTQPHSVSESPG | SEQ |
| F03A | GSLRLSCAASGFTFS | ID | KTVTISCTRSSGSIA | ID |
| | SYSMNWVRQAPGKGL | NO: | SNYVQWYQQRPGSSP | NO: |
| | EWVSYISSSSSTIYY | 172 | TTVIYEDNQRPSGVP | 230 |
| | ADSVKGRFTISRDNA | | DRFSGSIDSSSNSAS | |
| | KNSLYLQMNSLRDED | | LTISGLKTEDEADYY | |
| | TAVYYCARVRRNYDM | | CQSYDSSNHWVFGGG | |
| | DVWGQGTTVTVSS | | TKLTVL | |
| 70701_23 | QVQLRESGPGLVKPS | SEQ | NFMLTQPHSVSESPG | SEQ |
| N16A | QTLSLTCTVSGGSIN | ID | KTVTISCTRSSGSIA | ID |
| | SGGYCWSWIRQHPGK | NO: | SNYVQWYQQRPGSSP | NO: |
| | GLEWIGSIYYSGSTY | 173 | TTVIYEDNQRPSGVP | 231 |
| | YNPSLKSRVTISVDT | | DRFSGSIDSSSNSAS | |
| | SKNQFSLKLSSVTAA | | LTISGLKTEDEADYY | |
| | DTAVYYCARERKYLL | | CQSFDNSNRVFAGGT | |
| | YFDYWGQGTLVTVSS | | KLTVL | |
| 70701_24 | EVQLVESGGGLVKPG | SEQ | NFMLTQPHSVSESPG | SEQ |
| B09A | GSLRLSCAASGFTFS | ID | KTVTISCTRSSGSIA | ID |
| | SYCMNWVRQAPGKGL | NO: | SNYVQWYQQRPGSSP | NO: |
| | EWVSSISRSSSYIYY | 174 | TTVIYEDNQRPSGVP | 232 |
| | ADSVKGRFTISRDNA | | DRFSGSIDSSSNSAS | |
| | KNSLYLQMNSLRAED | | LTISGLKTEDEADYY | |
| | TAVYYCSRERWNLLF | | CQSYDSSNRVFGGGT | |
| | FDYWGQGTLVTVSS | | KLTVL | |
| 70701_24 | EVQLVESGGGLVKPG | SEQ | NFMLTQPHSVSESPG | SEQ |
| N03A | GSLRLSCAASGFTFS | ID | KTVTISCTRSSGSIA | ID |
| | SYSINWVRQAPGKGL | NO: | SNYVQWYQQRPGCSP | NO: |
| | EWVSSISRSSSYIYY | 175 | TTVIYEDNQRPSGVP | 233 |
| | ADSVKGRFTISRDNA | | DRFSGSIDSSSNSAS | |
| | KNSLYLQMNSLRAED | | LTISGLKTEDEADYY | |
| | TAVYYCARELWELLY | | CQSYDSNNRVFGGGT | |
| | FDYWGQGTLVTVSS | | KLTVL | |

TABLE 3
identified somatic hypermutations in the CD3 antibodies

| | Heavy Chain | | | | | | | | |
|---|---|---|---|---|---|---|---|---|---|
| | | | | | | | | | CDR |
| | | N- | | Isomerisation | Isomerisation | Aberrant | Missing | Integrin | Met and |
| Antibody | SHM | Glyoslyation | Deamidation | (DG/DS) | (DT/DD/DH) | Cysteines | Cysteines | Binding | Trp |
| 70701_05C15A | S31R A40T S53N | | NS NS NS | DS DS | DT | c54 | | | CDR3 |
| | S55C S56N N74S | | | | | | | | |
| 70701_05D06A | A23V S31N S35N | | NS NS | DG DS | DT | | | | CDR1 |
| | Q53K | | | | | | | | CDR3 |
| 70701_05L17A | T28N F29L S31T | NL | NS NS | | DT | | | | CDR3 |
| | S53R S63A K76Q | | | | | | | | |
| 70701_05M03A | T23S L29F S30T | | | | DT DT DD | c48 | | | CDR2 |
| | W49C S67N Y96F | | | | | | | | CDR3 |
| 70701_06A03A | Q3L A23E S31R | | NS | DS | DT | | | | CDR3 |
| | S53R S56R N84I | | | | | | | | |
| | Y94F | | | | | | | | |
| 70701_06A16A | F29V S30N S31N | NY | NS NS | DS | DT | | | | CDR3 |
| | S53R S56N | | | | | | | | |
| 70701_06B15A | T28N F29L S31T | NL | NS NS | DS | DT | | | | CDR3 |
| | S53R K76Q | | | | | | | | |
| 70701_06M17A | S49A I51M S53R | | NS NS | | DT | | | | CDR2 |
| | D62H M83L | | | | | | | | CDR3 |
| 70701_07N16A | F29L S33N N35D | | NS NS | DS | DT | | | | CDR3 |
| | S53R S56N K76E | | | | | | | | |
| 70701_08J03A | S30R S53R | | NS NS | DS | DT | | | | CDR3 |
| 70701_09C02A | S31R A40T S56N | | NS NS | DS | DT | | | | CDR3 |
| 70701_09K17A | V12G S53R T69I | | NS NS | DS | DT | | | | CDR3 |
| | N74K | | | | | | | | |
| 70701_10D02A | F29L S50T I51M | | NS NS | DS | DT | | | | CDR2 |
| | S53R | | | | | | | | CDR3 |
| 70701_10F19A | F29I S31R S53R | | NS NS | DS | DT | | | | CDR3 |
| | Y57H L79M | | | | | | | | |
| 70701_10J09A | F29L N35D S53R | | NS NS | DS | DT | | | | CDR3 |
| | S56N K76E | | | | | | | | |
| 70701_10N20A | S7F K13R A23V | | NS NS | DS | DT | | | | CDR3 |
| | S31I Y32F S33T | | | | | | | | |
| | S50T I51S S53R | | | | | | | | |
| | S55T | | | | | | | | |
| 70701_11B10A | G56A | | | | DT | | | | CDR3 |
| 70701_11K23A | S33G S53R S56R | | NS NS | DG DS | DT | | | | CDR3 |
| | I58R A92S | | | | | | | | |
| 70701_11L01A | S31R M34I V64L | | NS NS | DS | DT | | | | CDR3 |
| | K76R | | | | | | | | |
| 70701_11M11A | S31R M34I V64L | | NS NS | DS | DT | | | | CDR3 |
| | K76R | | | | | | | | |
| 70701_12F16A | S7F K13R A23V | | NS NS | DS | DT | | | | CDR3 |
| | S31I Y32F S33T | | | | | | | | |
| | S50T I51S | | | | | | | | |
| | S53R S55T | | | | | | | | |
| 70701_12J12A | S31R M34I I51S | | NS NS | DS | DT | | | | CDR3 |
| | S53R | | | | | | | | |
| 70701_12O16A | A23G S56R V64L | | NS NS | DS | DT | | | | CDR3 |
| | K65R | | | | | | | | |
| 70701_13C01A | S31R S53G S56R | | NS | DS | DT | | | | |
| | I70V N84S R87S | | | | | | | | |
| | A88P | | | | | | | | |
| 70701_13G21A | S31I S53K S54T | | NS NS | DS | DT | | | | |
| | S56T Y57F Y59F | | | | | | | | |
| | R87S | | | | | | | | |
| 70701_13H15A | S31R M34L S56R | | NS NS | DS | DT | | | | |
| | Y59F K65Q Y95F | | | | | | | | |
| 70701_14F08A | S31R S56R Y59F | | NS NS | DS | DT | | | | |
| 70701_14F21A | S31R S56R Y59F | | NS | DS | DT | | | | |
| | I70M S85N | | | | | | | | |
| 70701_14H05A | Q3H S31R S56R | | NS NS | DS | DT | | | | |
| 70701_14K01A | S31R M34L S56R | | NS NS | DS | DT | | | | |
| 70701_14K02A | P14R A24T F29L | | NS NS | DS | DT | | | | |
| | S31R V93L | | | | | | | | |
| 70701_14M09A | S31I S53K S56R | | NS NS | DS DS | DT | | | | |
| | Y59F R87S | | | | | | | | |
| 70701_16D18A | L11S S21T S31T | | NS NS | DS | DT | | | | CDR3 |
| | S53R | | | | | | | | |
| 70701_16E13A | S53R S55T | | NS NS | DS | DT | | | | CDR3 |
| 70701_16L13A | S53R | | NS NS | DS | DT | | | | CDR3 |
| 70701_16P13A | I51S S53R S71T | | NS NS | DS | DT | | | | CDR3 |
| 70701_17A23A | S53R S56N K76R | | NS NT | DS | | | | | CDR3 |
| | N84S T91A | | | | | | | | |
| 70701_17G08A | S55G N77S | | NS | DS | DT | | | | CDR3 |
| 70701_17H17A | A23V A24V S55H | | NS NS | DS | DT | | | | CDR3 |
| | F68L | | | | | | | | |
| 70701_18A16A | S55I S56N | | NS NS NT | DS | DT | | | | CDR3 |
| 70701_18E19A | M34V S53R T57I | | NS NS | DS | DT | | | | CDR3 |
| | A61V | | | | | | | | |
| 70701_18M14A | T28I F29L S30N | NY | NS NS | DS | DT DD | | | | CDR3 |
| | S31N S53R E89D | | | | | | | | |
| 70701_19A10A | V2A S53R V64A | | NS NS | DS | DT | | | | CDR3 |
| 70701_19C22A | G16E S53R S56N | | NS NS NT | DS | DT | | | | CDR3 |
| | K76R | | | | | | | | |
| 70701_19D03A | A24V I51S L79V | | NS NS | DS | DT | | | | CDR3 |
| 70701_19E13A | Q3H F27I S31R | | NS NS | DS | DT | | | | CDR3 |
| | S53R S56N | | | | | | | | |
| 70701_19I16A | S30T G33A Y34S | | | | DT DT | | | | |
| | Y61H N62S Q79R | | | | | | | | |
| | K83M | | | | | | | | |
| 70701_19J10A | V12M G33V S37N | | | | DT DT | | | | |
| | Y52D A98V | | | | | | | | |
| 70701_19O15A | Y80F | | NS NS | DS | DT | | | | CDR3 |
| 70701_20O18A | F27L S31R E46Y | | NS NS | DS | DT | | | | CDR3 |
| | S53T S56T Y80S | | | | | | | | |
| 70701_21A06A | K13E T21I G33A | | | | DT DT | | | | |
| | Y34S S58T Y61F | | | | | | | | |
| | V73L | | | | | | | | |
| 70701_21J18A | G33A Y35C Y52S | | | | DT DT | c34 | | | |
| | N78K | | | | | | | | |
| 70701_22D03A | V2M P14R G26K | | NS NS | DS | DT | | | | CDR3 |
| | S31G S33A S55T | | | | | | | | |
| | I58T | | | | | | | | |
| 70701_22O09A | S31R M34I S54N | NT | NS NS NT | DS | DT | | | | CDR3 |
| | S55T | | | | | | | | |
| 70701_23F03A | E6G | | NS NS | DS | DT | | | | CDR3 |
| 70701_23N16A | Q5R S30N Y35C | | NS | | DT DT | c34 | | | |
| | Y52S | | | | | | | | |
| 70701_24B09A | S33C S53R A97S | | NS NS | DS | DT | c32 | | | CDR3 |
| 70701_24N03A | M34I S53R | | NS NS | DS | DT | | | | CDR3 |
| | Corresponding Light Chain Sequences | | | | | | | | |
| | | | | | | | | | CDR |
| | | N- | | Isomerisation | Isomerisation | Aberrant | Missing | Integrin | Met and |
| Antibody | SHM | Glyoslyation | Deamidation | (DG/DS) | (DT/DD/DH) | Cysteines | Cysteines | Binding | Trp |
| 70701_05C15A | I49V D68A | | NS | DS | | | | | |
| 70701_05D06A | D29H A32V S5IN | | NS NT | DS | | c33 | | | CDR3 |
| 70701_05L17A | F2V T5I N32K | | NS | DS DS | | | | | |
| 70701_05M03A | P7S | | NT | | | c91 | | | |
| 70701_06A03A | R19S S51T | | NS NT | DS | DT DD DH | | | | CDR3 |
| 70701_06A16A | T47P N53Y Y94F | | NS | DS | | | | | |
| | S96R | | | | | | | | |
| 70701_06B15A | F2V T5I N32K | | NS | DS DS | | | | | |
| 70701_06M17A | S28N S31R | | NS | DS DS | | | | | |
| 70701_07N16A | Y33F N72K | | | DS DS | | | | | |
| 70701_08J03A | G67E S93T | | NS NT | DS DS | DD DH | | | | CDR3 |
| 70701_09C02A | I49V D68A | | NS | DS | | | | | |
| 70701_09K17A | I20F E51V | | NS | DS DS | | | | | CDR3 |
| 70701_10D02A | | | NS | DS DS | | | | | |
| 70701_10F19A | F2V P7T | | NS | DS DS | | | | | |
| 70701_10J09A | Y33F | | NS | DS DS | | | | | |
| 70701_10N20A | | | NS | DS DS | | | | | |
| 70701_11B10A | N53Y | | | DS | | | | | CDR3 |
| 70701_11K23A | S92T | | | DS | DT | | | | |
| 70701_11L01A | S96G | | NS | DG DS | | | | | |
| 70701_11M11A | S96G | | NS | DG DS | | | | | |
| 70701_12F16A | | | NS | DS DS | | | | | |
| 70701_12J12A | S96R | | NS | DS | | | | | |
| 70701_12O16A | S28N | | NS | DS DS | | | | | |
| 70701_13C01A | S31N Y33F Y37F | | NS | DS DS | | | | | |
| | T47D | | | | | | | | |
| 70701_13G21A | Y90S D95N S96G | | NG NS | DS | | | | | |
| | S97N | | | | | | | | |
| 70701_13H15A | T47P Y90F S96N | | NS NS | DS | | | | | |
| 70701_14F08A | S28N T47A S96G | | NS | DG DS | | | | | |
| 70701_14F21A | S96N | | NS NS | DS | | | | | |
| 70701_14H05A | | | NS | DS DS | | | | | |
| 70701_14K01A | N53Y D61A S96N | | NS | DS | | | | | |
| | S97N | | | | | | | | |
| 70701_14K02A | T46A S95N | | NS NS | DS | | | | | |
| 70701_14M09A | N53Y Y90S S96G | | NS | DG DS | | | | | |
| | S97N | | | | | | | | |
| 70701_16D18A | E51D | | NS | DS DS | DD | | | | CDR3 |
| 70701_16E13A | E51D D88E | | NS | DS DS | DD | | | | CDR3 |
| 70701_16L13A | Y37F E51D G80R | | NS | DS DS | DD | | | | CDR3 |
| 70701_16P13A | | | NS | DS DS | | | | | |
| 70701_17A23A | E51D S97N | | NS | DS DS | DD | | | | CDR3 |
| 70701_17G08A | E51D N72S | | | DS DS | DD | | | | CDR3 |
| 70701_17H17A | N1K F2L S31R | | NS | DS DS | DD DH | | | | CDR3 |
| | E51D K82R N98D | | | | | | | | |
| 70701_18A16A | S28N S31R S97G | | NS | DS DS | | | | | CDR3 |
| 70701_18E19A | E51D S97N | | NS | DS DS | DD | | | | CDR3 |
| 70701_18M14A | V18I S25R S26N | | NG NS | DS DS | | | | | |
| | S28N S43R | | | | | | | | |
| 70701_19A10A | E50D | | NS | DS DS | DD | | | | CDR3 |
| 70701_19C22A | E51D | | NS | DS DS | DD | | | | CDR3 |
| 70701_19D03A | S31T V48L E51D | | NS | DS DS | DD | | | | CDR3 |
| 70701_19E13A | | | NS | DS DS | | | | | |
| 70701_19I16A | N53S | | NS | DS DS DS | | | | | |
| 70701_19J10A | Y37F Y50F | | NS | DS DS | | | | | |
| 70701_19O15A | M3V T5P T47S | | NS | DS DS DS | DD | | | | CDR3 |
| | E51D N53S K82T | | | | | | | | |
| 70701_20O18A | E51D | | NS | DS DS | DD | | | | CDR3 |
| 70701_21A06A | Q39L Y94F | | NS | DS DS | | | | | |
| 70701_21J18A | I29V N53Y S96R | | NS | DS | | | | | |
| 70701_22D03A | E50D | | NS | DS DS | DD | | | | CDR3 |
| 70701_22O09A | E51D | | NS | DS DS | DD | | | | CDR3 |
| 70701_23F03A | | | NS | DS DS | | | | | CDR3 |
| 70701_23N16A | Y94F S96N | | NS NS | DS | | | | | |
| 70701_24B09A | | | NS | DS DS | | | | | |
| 70701_24N03A | S43C S97N | | NS | DS DS | | c42 | | | |

TABLE 4
variable region sequence clusters

| Clus- | Identi- | Anti- | Iso- | V | V % | D | D % | J | J % | | Iso- | V | V % | J | J % | |
|---|---|---|---|---|---|---|---|---|---|---|---|---|---|---|---|---|
| ter | cals | body | type | Gene | Match | Gene | Match | Gene | Match | CDR3 | type | Gene | Match | Gene | Match | CDR3 |
| Clust | | 70701_ | IGHG | IGHV | 95.2% | IGHD | 100.0% | IGHJ | 97.8% | ARFS | λ | IGLV | 99.0% | IGLJ | 100.0% | QVWD |
| 1.1 | | 106A03A | 1 | 3-21 | | 7-27 | | 4 | | NWGS | | 3-21 | | 2 | | SSSD |
| | | | | | | | | | | GGYF | | | | | | HPV |
| | | | | | | | | | | DY | | | | | | |
| Clust | | 70701_ | IGHG | IGHV | 99.0% | IGHD | 100.0% | IGHJ | 97.8% | ARFS | λ | IGLV | 99.3% | IGLJ | 100.0% | QVWD |
| 1.2 | | 108J03A | 2a | 3-21 | | 7-27 | | 4 | | NWGS | | 3-21 | | 2 | | STSD |
| | | | | | | | | | | GGYF | | | | | | HPV |
| | | | | | | | | | | DY | | | | | | |
| Clust | | 70701_ | IGHG | IGHV | 96.6% | IGHD | 100.0% | IGHJ | 100.0% | ARMR | λ | IGLV | 99.0% | IGLJ | 100.0% | SYDS |
| 2.1 | | 105C15A | 1 | 3-21 | | 2-2 | | 4 | | YNYF | | 6-57 | | 2 | | SNVV |
| | | | | | | | | | | DY | | | | | | F |
| Clust | | 70701_ | IGHG | IGHV | 98.0% | IGHD | 100.0% | IGHJ | 100.0% | ARMR | λ | IGLV | 99.0% | IGLJ | 100.0% | SYDS |
| 2.2 | | 109C02A | 1 | 3-21 | | 2-2 | | 4 | | YNYF | | 6-57 | | 2 | | SNVV |
| | | | | | | | | | | DY | | | | | | F |
| Clust | Identi- | 70701_ | IGHG | IGHV | 97.6% | IGHD | 100.0% | IGHJ | 97.9% | AKMR | λ | IGLV | 99.0% | IGLJ | 100.0% | SYDG |
| 2.3 | cal_2 | 11L01A | 1 | 3-21 | | 2-2 | | 4 | | YNYF | | 6-57 | | 2 | | SNVV |
| | | | | | | | | | | DY | | | | | | F |
| Clust | Identi- | 70701_ | IGHG | IGHV | 97.6% | IGHD | 100.0% | IGHJ | 97.9% | AKMR | λ | IGLV | 99.0% | IGLJ | 100.0% | SYDG |
| 2.3 | cal_2 | 11M11A | 1 | 3-21 | | 2-2 | | 4 | | YNYF | | 6-57 | | 2 | | SNVV |
| | | | | | | | | | | DY | | | | | | F |
| Clust | | 70701_ | IGHG | IGHV | 98.3% | IGHD | 100.0% | IGHJ | 100.0% | ARMR | λ | IGLV | 99.3% | IGLJ | 97.3% | SYDS |
| 2.4 | | 12016A | 1 | 3-21 | | 5-24 | | 4 | | YNYF | | 6-57 | | 2 | | SNVV |
| | | | | | | | | | | DY | | | | | | F |
| Clust | | 70701_ | IGHG | IGHV | 97.6% | IGHD | 100.0% | IGHJ | 97.8% | AREQ | λ | IGLV | 97.3% | IGLJ | 100.0% | SYDS |
| 3.1 | | 105L17A | 1 | 3-21 | | 1-14 | | 4 | | WEPL | | 6-57 | | 3 | | SNRV |
| | | | | | | | | | | YFDY | | | | | | F |
| Clust | | 70701_ | IGHG | IGHV | 98.3% | IGHD | 100.0% | IGHJ | 100.0% | ARER | λ | IGLV | 99.7% | IGLJ | 100.0% | SYDS |
| 3.10 | | 19E13A | 2a | 3-21 | | 1-26 | | 4 | | WELL | | 6-57 | | 3 | | SNRV |
| | | | | | | | | | | YFDY | | | | | | F |
| Clust | | 70701_ | IGHG | IGHV | 98.6% | IGHD | 91.7% | IGHJ | 100.0% | AREL | λ | IGLV | 99.0% | IGLJ | 100.0% | SYDS |
| 3.11 | | 124N03A | 1 | 3-21 | | 1-26 | | 4 | | WELL | | 6-57 | | 3 | | NNRV |
| | | | | | | | | | | YFDY | | | | | | F |
| Clust | | 70701_ | IGHG | IGHV | 97.6% | IGHD | 92.3% | IGHJ | 100.0% | AREK | λ | IGLV | 97.3% | IGLJ | 100.0% | SYDS |
| 3.2 | | 106B15A | 1 | 3-21 | | 1-26 | | 4 | | WELL | | 6-57 | | 3 | | SNRV |
| | | | | | | | | | | YFDY | | | | | | F |
| Clust | | 70701_ | IGHG | IGHV | 98.3% | IGHD | 100.0% | IGHJ | 100.0% | ARER | λ | IGLV | 98.6% | IGLJ | 100.0% | SYDS |
| 3.3 | | 106M17A | 2b | 3-21 | | 1-26 | | 4 | | WELL | | 6-57 | | 3 | | SNRV |
| | | | | | | | | | | FFDY | | | | | | F |
| Clust | | 70701_ | IGHG | IGHV | 98.0% | IGHD | 100.0% | IGHJ | 100.0% | ARER | λ | IGLV | 99.7% | IGLJ | 100.0% | SYDS |
| 3.4 | | 10D02A | 2a | 3-21 | | 1-26 | | 4 | | WELL | | 6-57 | | 3 | | SNRV |
| | | | | | | | | | | FFDY | | | | | | F |
| Clust | | 70701_ | IGHG | IGHV | 98.3% | IGHD | 100.0% | IGHJ | 100.0% | AREK | λ | IGLV | 98.6% | IGLJ | 100.0% | SYDS |
| 3.5 | | 10F19A | 1 | 3-21 | | 1-26 | | 4 | | WELL | | 6-57 | | 3 | | SNRV |
| | | | | | | | | | | YFDY | | | | | | F |
| Clust | Identi- | 70701_ | IGHG | IGHV | 94.9% | IGHD | 92.3% | IGHJ | 97.9% | AREQ | λ | IGLV | 99.3% | IGLJ | 100.0% | SYDS |
| 3.6 | cal_ | 10N20A | 1 | 3-21 | | 1-26 | | 4 | | WELL | | 6-57 | | 3 | | SNRV |
| | 1 | | | | | | | | | YFDY | | | | | | F |
| Clust | Identi- | 70701_ | IGHG | IGHV | 95.3% | IGHD | 92.3% | IGHJ | 97.9% | AREQ | λ | IGLV | 99.3% | IGLJ | 100.0% | SYDS |
| 3.6 | cal_ | 12F16A | 1 | 3-21 | | 1-26 | | 4 | | WELL | | 6-57 | | 3 | | SNRV |
| | 1 | | | | | | | | | YFDY | | | | | | F |
| Clust | | 70701_ | IGHG | IGHV | 98.6% | IGHD | 92.3% | IGHJ | 100.0% | AREQ | λ | IGLV | 99.3% | IGLJ | 100.0% | SYDR |
| 3.7 | | 12J12A | 1 | 3-21 | | 1-26 | | 4 | | WELL | | 6-57 | | 3 | | SNRV |
| | | | | | | | | | | YFDY | | | | | | F |
| Clust | | 70701_ | IGHG | IGHV | 99.0% | IGHD | 100.0% | IGHJ | 100.0% | ARER | λ | IGLV | 99.7% | IGLJ | 100.0% | SYDS |
| 3.8 | | 16P13A | 1 | 3-21 | | 1-26 | | 4 | | WELL | | 6-57 | | 3 | | SNRV |
| | | | | | | | | | | YFDY | | | | | | F |
| Clust | | 70701_ | IGHG | IGHV | 96.3% | | | IGHJ | 100.0% | AREQ | λ | IGLV | 97.6% | IGLJ | 100.0% | SYDS |
| 3.9 | | 18M14A | 2a | 3-21 | | | | 4 | | WVLL | | 6-57 | | 3 | | SNRV |
| | | | | | | | | | | YFDY | | | | | | F |
| Clust | | 70701_ | IGHG | IGHV | 97.3% | IGHD | 100.0% | IGHJ | 97.8% | AREK | λ | IGLV | 99.0% | IGLJ | 100.0% | SYDS |
| 4.1 | | 107N16A | 2a | 3-21 | | 1-1 | | 4 | | WVQL | | 6-57 | | 3 | | SNRV |
| | | | | | | | | | | YFDF | | | | | | F |
| Clust | | 70701_ | IGHG | IGHV | 97.6% | IGHD | 100.0% | IGHJ | 97.8% | AREK | λ | IGLV | 99.3% | IGLJ | 100.0% | SYDS |
| 4.2 | | 10J09A | 2a | 3-21 | | 1-1 | | 4 | | WVQL | | 6-57 | | 3 | | SNRV |
| | | | | | | | | | | YFDF | | | | | | F |
| Clust | | 70701_ | IGHG | IGHV | 97.0% | IGHD | 100.0% | IGHJ | 100.0% | ARER | λ | IGLV | 97.6% | IGLJ | 100.0% | SYDS |
| 5.1 | | 13C01A | 2a | 3-21 | | 1-14 | | 4 | | YNYF | | 6-57 | | 3 | | SNRV |
| | | | | | | | | | | DY | | | | | | F |
| Clust | | 70701_ | IGHG | IGHV | 96.6% | IGHD | 100.0% | IGHJ | 100.0% | ARER | λ | IGLV | 98.0% | IGLJ | 100.0% | SYNG |
| 5.2 | | 13G21A | 1 | 3-21 | | 1-14 | | 4 | | YNYF | | 6-57 | | 3 | | NNRV |
| | | | | | | | | | | DY | | | | | | F |
| Clust | | 70701_ | IGHG | IGHV | 97.0% | IGHD | 100.0% | IGHJ | 100.0% | ARER | λ | IGLV | 98.6% | IGLJ | 100.0% | SYDN |
| 5.3 | | 13H15A | 1 | 3-21 | | 1-14 | | 4 | | YNYF | | 6-57 | | 3 | | SNRV |
| | | | | | | | | | | DY | | | | | | F |
| Clust | | 70701_ | IGHG | IGHV | 98.0% | IGHD | 100.0% | IGHJ | 97.9% | ARER | λ | IGLV | 98.3% | IGLJ | 100.0% | SYDG |
| 5.4 | | 14F08A | 2b | 3-21 | | 1-14 | | 4 | | YNYF | | 6-57 | | 3 | | SNRV |
| | | | | | | | | | | DF | | | | | | F |
| Clust | | 70701_ | IGHG | IGHV | 98.3% | IGHD | 100.0% | IGHJ | 97.9% | AREP | λ | IGLV | 99.3% | IGLJ | 100.0% | SYDN |
| 5.5 | | 14F21A | 1 | 3-21 | | 2-2 | | 4 | | YNYF | | 6-57 | | 3 | | SNRV |
| | | | | | | | | | | DY | | | | | | F |
| Clust | | 70701_ | IGHG | IGHV | 99.0% | IGHD | 100.0% | IGHJ | 100.0% | ARER | λ | IGLV | 99.7% | IGLJ | 100.0% | SYDS |
| 5.6 | | 14H05A | 2b | 3-21 | | 1-14 | | 4 | | YNYF | | 6-57 | | 3 | | SNRV |
| | | | | | | | | | | DY | | | | | | F |
| Clust | | 70701_ | IGHG | IGHV | 98.6% | IGHD | 100.0% | IGHJ | 100.0% | ARER | λ | IGLV | 98.3% | IGLJ | 97.1% | SYDN |
| 5.7 | | 14K01A | 1 | 3-21 | | 1-14 | | 4 | | YNYF | | 6-57 | | 3 | | NNRV |
| | | | | | | | | | | DY | | | | | | F |
| Clust | | 70701_ | IGHG | IGHV | 98.0% | IGHD | 100.0% | IGHJ | 100.0% | ARER | λ | IGLV | 99.0% | IGLJ | 100.0% | SYDN |
| 5.8 | | 14K02A | 1 | 3-2 | | 1-14 | | 4 | | YNYF | | 6-57 | | 3 | | SNRV |
| | | | | | | | | | | DY | | | | | | F |
| Clust | | 70701_ | IGHG | IGHV | 97.3% | IGHD | 100.0% | IGHJ | 97.8% | ARER | λ | IGLV | 98.0% | IGLJ | 100.0% | SYDG |
| 5.9 | | 14M09A | 1 | 3-21 | | 3-16 | | 4 | | YNYF | | 6-57 | | 3 | | NNRV |
| | | | | | | | | | | DS | | | | | | F |
| Clust | | 70701_ | IGHG | IGHV | 97.6% | IGHD | 100.0% | IGHJ | 100.0% | ARVV | λ | IGLV | 99.0% | IGLJ | 100.0% | SYDS |
| 6.1 | | 109K17A | 1 | 3-48 | | 6-13 | | 6 | | RDYG | | 6-57 | | 3 | | SNHW |
| | | | | | | | | | | MDV | | | | | | VF |
| Clust | | 70701_ | IGHG | IGHV | 98.6% | | | IGHJ | 100.0% | ARVR | λ | IGLV | 99.3% | IGLJ | 100.0% | YDSS |
| 6.10 | | 19A10A | 1 | 3-48 | | | | 6 | | RNYG | | 6-57 | | 3 | | NHWV |
| | | | | | | | | | | MDV | | | | | | FG |
| Clust | | 70701_ | IGHG | IGHV | 98.3% | | | IGHJ | 100.0% | ARVR | λ | IGLV | 99.3% | IGLJ | 100.0% | SYDS |
| 6.11 | | 19C22A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 98.6% | | | IGHJ | 100.0% | ARVR | λ | IGLV | 98.3% | IGLJ | 100.0% | SYDS |
| 6.12 | | 19D03A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 99.3% | | | IGHJ | 100.0% | ARVR | λ | IGLV | 97.6% | IGLJ | 100.0% | SYDS |
| 6.13 | | 19015A | 1 | 3-48 | | | | 6 | | RNYG | | 6-57 | | 3 | | SNWV |
| | | | | | | | | | | MDV | | | | | | F |
| Clust | | 70701_ | IGHG | IGHV | 96.6% | | | IGHJ | 100.0% | ARVR | λ | IGLV | 99.3% | IGLJ | 97.4% | SYDS |
| 6.14 | | 120018A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 95.6% | | | IGHJ | 100.0% | ARVR | λ | IGLV | 99.3% | IGLJ | 100.0% | SYDS |
| 6.15 | | 122D03A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 98.3% | | | IGHJ | 100.0% | ARVR | λ | IGLV | 99.3% | IGLJ | 100.0% | SYDS |
| 6.16 | | 122009A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 99.7% | | | IGHJ | 98.0% | ARVR | λ | IGLV | 99.7% | IGLJ | 100.0% | SYDS |
| 6.17 | | 123F03A | 2a | 3-48 | | | | 6 | | RNYD | | 6-57 | | 3 | | SNHW |
| | | | | | | | | | | MDV | | | | | | VF |
| Clust | | 70701_ | IGHG | IGHV | 98.3% | | | IGHJ | 100.0% | ARVR | λ | IGLV | 98.6% | IGLJ | 100.0% | SYDS |
| 6.2 | | 16D18A | 2a | 3-48 | | | | 6 | | RNYG | | 6-57 | | 3 | | SNHW |
| | | | | | | | | | | MDV | | | | | | VF |
| Clust | | 70701_ | IGHG | IGHV | 99.0% | | | IGHJ | 98.0% | ARVR | λ | IGLV | 99.0% | IGLJ | 100.0% | SYDS |
| 6.3 | | 16E13A | 1 | 3-48 | | | | 6 | | RNYG | | 6-57 | | 3 | | SNHW |
| | | | | | | | | | | MDV | | | | | | VF |
| Clust | | 70701_ | IGHG | IGHV | 99.0% | | | IGHJ | 100.0% | ARVR | λ | IGLV | 98.6% | IGLJ | 100.0% | SYDS |
| 6.4 | | 16L13A | 1 | 3-48 | | | | 6 | | RNYG | | 6-57 | | 3 | | SNWV |
| | | | | | | | | | | MDV | | | | | | F |
| Clust | | 70701_ | IGHG | IGHV | 98.3% | | | IGHJ | 100.0% | ARVR | λ | IGLV | 99.0% | IGLJ | 100.0% | SYDS |
| 6.5 | | 17A23A | 1 | 3-48 | | | | 6 | | RNYG | | 6-57 | | 3 | | NNHW |
| | | | | | | | | | | MDV | | | | | | VF |
| Clust | | 70701_ | IGHG | IGHV | 99.3% | | | IGHJ | 100.0% | ARVR | λ | IGLV | 99.0% | IGLJ | 100.0% | SYDS |
| 6.6 | | 17G08A | 2a | 3-48 | | | | 6 | | RNYG | | 6-57 | | 3 | | SNHW |
| | | | | | | | | | | MDV | | | | | | VF |
| Clust | | 70701_ | IGHG | IGHV | 96.9% | | | IGHJ | 100.0% | ARVR | λ | IGLV | 97.3% | IGLJ | 100.0% | SYDS |
| 6.7 | | 17H17A | 1 | 3-48 | | | | 6 | | RNYG | | 6-57 | | 3 | | SDHW |
| | | | | | | | | | | MDV | | | | | | VF |
| Clust | | 70701_ | IGHG | IGHV | 99.3% | | | IGHJ | 98.0% | ARVR | λ | IGLV | 98.6% | IGLJ | 97.3% | SYDS |
| 6.8 | | 18A16A | 1 | 3-48 | | | | 6 | | RNYD | | 6-57 | | 3 | | GNWV |
| | | | | | | | | | | MDV | | | | | | F |
| Clust | | 70701_ | IGHG | IGHV | 96.9% | | | IGHJ | 100.0% | ARVR | λ | IGLV | 98.6% | IGLJ | 97.3% | SYDS |
| 6.9 | | 18E19A | 1 | 3-48 | | | | 6 | | RNYG | | 6-57 | | 3 | | NNWV |
| | | | | | | | | | | MDV | | | | | | F |
| Clust | | 70701_ | IGHG | IGHV | 97.7% | | | IGHJ | 100.0% | ARER | λ | IGLV | 99.0% | IGLJ | 97.2% | SYDS |
| 7.1 | | 19116A | 1 | 4-31 | | | | 4 | | KYPL | | 6-57 | | 3 | | SNRV |
| | | | | | | | | | | QFDY | | | | | | F |
| Clust | | 70701_ | IGHG | IGHV | 97.0% | | | IGHJ | 100.0% | ARER | λ | IGLV | 98.3% | IGLJ | 100.0% | SFDS |
| 7.2 | | 121A06A | 1 | 4-31 | | | | 4 | | KYPL | | 6-57 | | 3 | | SNRV |
| | | | | | | | | | | QFDY | | | | | | F |
| Clust | | 70701_ | IGHG | IGHV | 98.3% | | | IGHJ | 100.0% | ARER | λ | IGLV | 98.6% | IGLJ | 100.0% | SYDR |
| 8.1 | | 21J18A | 1 | 4-31 | | | | 4 | | KYLL | | 6-57 | | 3 | | SNRV |
| | | | | | | | | | | YFDY | | | | | | F |
| Clust | | 70701_ | IGHG | IGHV | 98.0% | | | IGHJ | 100.0% | ARER | λ | IGLV | 99.0% | IGLJ | 97.2% | SFDN |
| 8.2 | | 123N16A | 1 | 4-31 | | | | 4 | | KYLL | | 6-57 | | 3 | | SNRV |
| | | | | | | | | | | YFDY | | | | | | F |
| | | 70701_ | IGHG | IGHV | 99.7% | IGHD | 100.0% | IGHJ | 100.0% | ARGG | λ | IGLV | 99.3% | IGLJ | 100.0% | GTWD |
| | | 11B10A | 2a | 1-69 | | 3-10 | | 6 | | VGRN | | 1-51 | | 2 | | SSLS |
| | | | | | | | | | | YYYY | | | | | | AVV |
| | | | | | | | | | | GMDV | | | | | | |
| | | 70701_ | IGHG | IGHV | 98.0% | IGHD | 100.0% | IGHJ | 100.0% | AHLA | λ | IGLV | 99.7% | IGLJ | 100.0% | CSYA |
| | | 105M03A | 2b | 2-5 | | 7-27 | | 4 | | HWGP | | 2-23 | | 2 | | GSST |
| | | | | | | | | | | YFDY | | | | | | LI |
| | | 70701_ | IGHG | IGHV | 97.3% | | | IGHJ | 97.7% | ARER | λ | IGLV | 98.0% | IGLJ | 100.0% | SFDR |
| | | 106A16A | 2a | 3-21 | | | | 4 | | WALL | | 6-57 | | 3 | | SNRV |
| | | | | | | | | | | FFEY | | | | | | F |
| | | 70701_ | IGHG | IGHV | 98.0% | IGHD | 83.3% | IGHJ | 100.0% | SRER | λ | IGLV | 99.7% | IGLJ | 100.0% | SYDS |
| | | 124B09A | 1 | 3-21 | | 1-26 | | 4 | | WNLL | | 6-57 | | 3 | | SNRV |
| | | | | | | | | | | FFDY | | | | | | F |
| | | 70701_ | IGHG | IGHV | 97.6% | IGHD | 100.0% | IGHJ | 100.0% | ARDG | λ | IGLV | 99.3% | IGLJ | 100.0% | QSAD |
| | | 11K23A | 1 | 3-48 | | 1-26 | | 3 | | RWEL | | 3-25 | | 3 | | TSGT |
| | | | | | | | | | | LAFD | | | | | | YRV |
| | | | | | | | | | | I | | | | | | |
| | | 70701_ | IGHG | IGHV | 98.0% | IGHD | 91.7% | IGHJ | 100.0% | ARVK | λ | IGLV | 98.2% | IGLJ | 100.0% | QAWD |
| | | 105D06A | 2a | 3-7 | | 1-26 | | 4 | | WDLL | | 3-1 | | 2 | | SSTH |
| | | | | | | | | | | DY | | | | | | VV |
| | | 70701_ | IGHG | IGHV | 96.3% | | | IGHJ | 100.0% | VRER | λ | IGLV | 98.3% | IGLJ | 100.0% | SYDS |
| | | 19J10A | 1 | 4-31 | | | | 4 | | KYPL | | 6-57 | | 3 | | SVRV |

1. An anti-CD3 antibody or antibody fragment comprising a heavy chain (VH) CDR3 sequence comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59.

2. The anti-CD3 antibody or antibody fragment of claim 1, wherein the antibody or fragment comprises a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

3. The anti-CD3 antibody or antibody fragment of claim 1, wherein the antibody or fragment comprises a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175.

4. The anti-CD3 antibody or antibody fragment of claim 1, wherein the antibody or fragment comprises a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

5. The anti-CD3 antibody or antibody fragment of claim 1, wherein the antibody or fragment comprises:
a) a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175; and
b) a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

6. An anti-CD3 antibody or antibody fragment comprising a heavy chain variable region and/or a light chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

7. The anti-CD3 antibody or antibody fragment of claim 6, wherein the antibody or antibody fragment comprises a heavy chain variable region comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

8. The anti-CD3 antibody or antibody fragment of claim 6, wherein the antibody or antibody fragment comprises a light chain variable region comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

9. The anti-CD3 antibody or antibody fragment of claim 6, wherein the antibody or antibody fragment comprises a heavy chain CDR3 sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

10. The anti-CD3 antibody or antibody fragment of claim 6, wherein the antibody or antibody fragment comprises a light chain CDR3 sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

11. A therapeutic composition comprising an anti-CD3 antibody or antibody fragment comprising a heavy chain (VH) CDR3 sequence comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59.

12. The therapeutic composition of claim 11, wherein the antibody or fragment comprises a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117.

13. The therapeutic composition of claim 11, wherein the antibody or fragment comprises a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175.

14. The therapeutic composition of claim 11, wherein the antibody or fragment comprises a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

15. The therapeutic composition of claim 11, wherein the therapeutic composition comprises a plurality of different CD3 antibodies, wherein each different CD3 antibody comprises a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175 and/or a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

16. The therapeutic composition of claim 11, wherein the antibody or fragment comprises a heavy chain variable region and/or a light chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

17. A method of treating a cancer patient with a therapeutic composition comprising at least one anti-CD3 antibody or fragment thereof, wherein said anti-CD3 antibody or fragment comprises:
a heavy chain (VH) CDR3 sequence comprising an amino acid sequence selected from one of SEQ ID NOS: 2-59, and/or a sequence that comprises an amino acid sequence having at least 85%, at least 90% at least 95%; at least 96%, at least 97%, at least 98% or at least 99% sequence identity with any of SEQ ID NOS: 2-59;
a light chain CDR3 sequence (VL or λ) comprising an amino acid sequence selected from one of SEQ ID NOS: 60-117, and/or a sequence that comprises an amino acid sequence having at least 85%, at least 90% at least 95%; at least 96%, at least 97%, at least 98% or at least 99% sequence identity with any of SEQ ID NOS: 60-117;
a heavy chain variable region comprising an amino acid sequence selected from one of SEQ ID NOS: 118-175, and/or a sequence that comprises an amino acid sequence having at least 85%, at least 90% at least 95%; at least 96%, at least 97%, at least 98% or at least 99% sequence identity with any of SEQ ID NOS: 118-175; and/or
a light chain variable region comprising an amino acid sequence selected from one of SEQ ID NOS: 176-233, and/or a sequence that comprises an amino acid sequence having at least 85%, at least 90% at least 95%; at least 96%, at least 97%, at least 98% or at least 99% sequence identity with any of SEQ ID NOS: 176-233.

18. The method of claim 17, wherein the least one anti-CD3 antibody or fragment thereof comprises a heavy chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

19. The method of claim 17, wherein the least one anti-CD3 antibody or fragment thereof comprises a light chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

20. The method of claim 17, wherein the least one anti-CD3 antibody or fragment thereof comprises a light chain variable region and heavy chain variable region sequence comprising the somatic hypermutations (SMH) of clusters: Clust 1.1; Clust 1.2; Clust 2.1; Clust 2.2; Clust 2.3; Clust 2.3; Clust 2.4; Clust 3.1; Clust 3.10; Clust 3.11; Clust 3.2; Clust 3.3; Clust 3.4; Clust 3.5; Clust 3.6; Clust 3.6; Clust 3.7; Clust 3.8; Clust 3.9; Clust 4.1; Clust 4.2; Clust 5.1; Clust 5.2; Clust 5.3; Clust 5.4; Clust 5.5; Clust 5.6; Clust 5.7; Clust 5.8; Clust 5.9; Clust 6.1; Clust 6.10; Clust 6.11; Clust 6.12; Clust 6.13; Clust 6.14; Clust 6.15; Clust 6.16; Clust 6.17; Clust 6.2; Clust 6.3; Clust 6.4; Clust 6.5; Clust 6.6; Clust 6.7; Clust 6.8; Clust 6.9; Clust 7.1; Clust 7.2; Clust 8.11 and/or Clust 8.2.

21.-30. (canceled)
